(19) United States
(12) Patent Application Publication
(10) Pub. No.: US 2026/0256369 A1
(43) Pub. Date: Sep. 3, 2026

(54) Optical Digit Probe for Noninvasive Measurement of Physiological Parameters

(71) Applicant: Itamar Medical Ltd., Caesarea (IL)

(72) Inventors: Nimrod Kadim, Modi'in-Maccabim-Re'ut (IL); Itay Gur-Arie, Atzmon-Segev (IL); Tom Kertesz, Pardes Hanna Karkur, Haifa (IL)

(21) Appl. No.: 19/163,031

(22) PCT Filed: Mar. 8, 2024
(86) PCT No.: PCT/IL2024/050253

§ 371 Date: Sep. 8, 2025

Related U.S. Application Data
(60) Provisional application No. 63/451,100, filed on Mar. 9, 2023.

(51) Int. Cl. A61B 5/022; A61B 5/00; A61B 5/024

(52) U.S. Cl. CPC A61B 5/02241; A61B 5/02427; A61B 5/6826; A61B 5/6838

(57) ABSTRACT

An optical digit probe for noninvasive measurement of at least one physiological parameter of a patient includes a digit housing having an external digit housing surface and an internal digit housing surface. A proximal end of the digit housing is adapted for facilitating insertion of a digit of a patient into the digit housing. The optical digit probe also includes an attachment device including a housing fastening portion connected to the external digit housing surface of the digit housing and a skin securement portion extending from the housing fastening portion configured to releasably couple the attachment device to a skin surface of the patient. The optical digit probe also includes at least one optical sensor disposed in the digit housing configured to be positioned proximate to the digit of the patient to provide optical signals reflecting the at least one physiological parameter of the patient.

### CROSS-REFERENCE TO RELATED APPLICATIONS

This application is the United States National Phase of International Patent Application No. PCT/IL2024/050253 filed Mar. 8, 2024, and claims priority to U.S. Provisional Patent Application No. 63/451,100 filed Mar. 9, 2023, the disclosures of which are hereby incorporated by reference in their entireties.

### BACKGROUND

### Field

The present disclosure relates to improved optical digit probes for noninvasive measurement of one or more physiological parameters of a wearer or patient.

### Description of Related Art

Optical digit probes, such as finger probes, are used for monitoring a variety of physiological signals or parameters of a patient including, for example, signals relating to breathing or respiratory parameters (e.g., blood oxygen levels, oxygen saturation, or oxygen uptake), as well as parameters for blood flow (e.g., heart rate or blood pressure) and blood composition (e.g., glucose concentration). Such probes can be used in connection with monitoring patients for certain sleep disordered breathing (SDB) conditions. For example, certain clinical, lab-based, or home-based sleep monitoring applications use such probes for monitoring the patient's physiological signals or parameters.

It is desirable therefore to improve the construction, design, and implementation of such optical digit probes to enhance their comfort and reliability over the duration of wear by the patient.

### SUMMARY

According to an aspect of the present disclosure, an optical digit probe for noninvasive measurement of at least one physiological parameter of a patient includes a digit housing having an external digit housing surface and an internal digit housing surface. A proximal end of the digit housing is adapted for facilitating insertion of a digit of a patient into the digit housing. The optical digit probe also includes an attachment device including a housing fastening portion connected to the external digit housing surface of the digit housing and a skin securement portion extending from the housing fastening portion configured to releasably couple the attachment device to a skin surface of the patient. The optical digit probe also includes at least one optical sensor disposed in the digit housing configured to be positioned proximate to the digit of the patient to provide optical signals reflecting the at least one physiological parameter of the patient.

According to another aspect of the present disclosure, an optical digit probe for noninvasive measurement of at least one physiological parameter of a patient includes a digit housing portion with an external digit housing surface and an internal digit housing surface. A proximal end of the digit housing portion is adapted for facilitating insertion of a digit of a patient into the digit housing portion. The optical digit probe also includes a skin securement portion configured to releasably couple the probe to a skin surface of the patient and an attachment portion extending between the external digit housing surface of the digit housing portion and the skin securement portion. The optical digit probe also includes at least one optical sensor disposed in the digit housing portion of the probe configured to be positioned proximate to the digit of the patient to provide optical signals reflecting the at least one physiological parameter of the patient.

According to another aspect of the present disclosure, an optical digit probe for noninvasive measurement of at least one physiological parameter of a patient includes a digit housing having an external digit housing surface and an internal digit housing surface. A proximal end of the digit housing is adapted for facilitating insertion of a digit of a patient into the digit housing. The optical digit probe also includes an attachment device removably connected to the external digit housing surface of the digit housing having a skin securement portion extending from the digit housing configured to releasably couple the attachment device to a skin surface of the patient. The optical digit probe also includes at least one optical sensor disposed in the digit housing configured to be positioned proximate to the digit of the patient to provide optical signals reflecting the at least one physiological parameter of the patient.

According to another aspect of the present disclosure, an optical digit probe for noninvasive measurement of at least one physiological parameter of a patient includes a digit housing having an external digit housing surface and an internal digit housing surface. A proximal end of the digit housing is adapted for facilitating insertion of at least portions of a first portion and a second portion of a digit of a patient into the digit housing. The optical digit probe also includes an attachment device having a housing fastening portion connected to the external digit housing surface of the digit housing and a skin securement portion configured to releasably couple the attachment device to a skin surface about a third portion of the digit of the patient. The optical digit probe also includes at least one optical sensor disposed in the digit housing configured to be positioned proximate to the digit of the patient to provide optical signals reflecting the at least one physiological parameter of the patient.

According to another aspect of the present disclosure, an optical digit probe for noninvasive measurement of at least one physiological parameter of a patient includes a digit housing having an external digit housing surface and an internal digit housing surface. A proximal end of the digit housing is adapted for facilitating insertion of a digit of a patient into the digit housing. The optical digit probe also includes an attachment device having a housing fastening portion connected to the external digit housing surface of the digit housing and a securement portion configured to be worn on a wrist, forearm, and/or arm of the patient for releasably coupling the attachment device to the patient. The optical digit probe also includes at least one optical sensor disposed in the digit housing configured to be positioned proximate to the digit of the patient to provide optical signals reflecting the at least one physiological parameter of the patient.

According to another aspect of the present disclosure, an optical digit probe for noninvasive measurement of at least one physiological parameter of a patient includes a digit housing having an external digit housing surface and an internal digit housing surface, wherein a proximal end of the digit housing is adapted for facilitating insertion of a digit of a patient into the digit housing and an attachment device connected to the external digit housing surface of the digit housing. The attachment device includes at least one anchor portion configured to be secured to at least one digit of the patient, the at least one digit being different from the digit inserted into the digit housing, thereby removably coupling the attachment device to the patient. The optical digit probe also includes at least one optical sensor disposed in the digit housing configured to be positioned proximate to the digit of the patient to provide optical signals reflecting the at least one physiological parameter of the patient.

According to a further aspect of the present disclosure, we also disclose, independently, the attachment device, the skin securement portion, and the securement portion of any of the above aspects separate from the remainder of the optical digit probe. It will be appreciated that in one or more examples, the attachment device, the skin securement portion, and the securement portion may be a replaceable part of the optical digit probe. In some examples, the attachment device, the skin securement portion, and the securement portion may be part of a digit probe that operates optically, including at infrared and/or visible wavelengths, and non-optically, such as by use of a radio frequency based or ultrasound based sensor(s).

According to a further aspect of the disclosure, we provide an optical digit probe for noninvasive measurement of at least one physiological parameter of a patient, the optical digit probe comprising:
a digit housing comprising an external digit housing surface and an internal digit housing surface, wherein a proximal end of the digit housing is adapted for facilitating insertion of a digit of the patient into the digit housing;
at least one optical sensor disposed in the digit housing configured to be positioned proximate to the digit of the patient to provide optical signals reflecting the at least one physiological parameter of the patient; and one of:
a) an attachment device comprising: a housing fastening portion connected to the external digit housing surface of the digit housing, and a skin securement portion extending from the housing fastening portion configured to releasably couple the attachment device to a skin surface of the patient;
b) a skin securement portion configured to releasably couple the probe to a skin surface of the patient; an attachment portion extending between the external digit housing surface of the digit housing portion and the skin securement portion;
c) an attachment device removably connected to the external digit housing surface of the digit housing comprising a skin securement portion extending from the digit housing configured to releasably couple the attachment device to a skin surface of the patient;
d) an attachment device having a housing fastening portion connected to the external digit housing surface of the digit housing and a skin securement portion configured to releasably couple the attachment device to a skin surface about a portion of the digit of the patient;
e) an attachment device having a housing fastening portion connected to the external digit housing surface of the digit housing and a securement portion configured to be worn on a wrist, forearm, and/or arm of the patient for releasably coupling the attachment device to the patient; or
f) an attachment device connected to the external digit housing surface of the digit housing, wherein the attachment device includes at least one anchor portion configured to be secured to at least one digit of the patient, the at least one digit being different from the digit inserted into the digit housing, thereby removably coupling the attachment device to the patient.

Preferred and non-limiting examples of the present disclosure will now be described in the following numbered clauses:
Clause 1: An optical digit probe for noninvasive measurement of at least one physiological parameter of a patient, the optical digit probe comprising: a digit housing comprising an external digit housing surface and an internal digit housing surface, wherein a proximal end of the digit housing is adapted for facilitating insertion of a digit of the patient into the digit housing; an attachment device comprising: a housing fastening portion connected to the external digit housing surface of the digit housing, and a skin securement portion extending from the housing fastening portion configured to releasably couple the attachment device to a skin surface of the patient; and at least one optical sensor disposed in the digit housing configured to be positioned proximate to the digit of the patient to provide optical signals reflecting the at least one physiological parameter of the patient.
Clause 2: The optical digit probe of clause 1, wherein the optical digit probe is configured for non-invasive measurement of arterial vasomotor activity through the digit of the patient.
Clause 3: The optical digit probe of clause 1 or clause 2, wherein the optical signals provided by the at least one optical sensor reflect an arterial pulse waveform of the patient.
Clause 4: The optical digit probe of any of clauses 1-3, wherein the attachment device comprises a longitudinal attachment device having a longitudinal axis corresponding to an insertion direction of the digit of the patient into the digit housing.
Clause 5: The optical digit probe of any of clauses 1-4, wherein the housing fastening portion is on a distal end of the attachment device and the skin securement portion is on a proximal end of the attachment device, the attachment device further comprising a middle portion between the housing fastening portion and the skin securement portion.
Clause 6: The optical digit probe of clause 4 or clause 5, wherein the longitudinal attachment device is configured to be permanently fastened to the digit housing.
Clause 7: The optical digit probe of any of clauses 4-6, wherein the longitudinal attachment device is configured to be releasably fastened to the digit housing.
Clause 8: The optical digit probe of any of clauses 1-7, wherein the digit housing comprises a pressure device configured to apply a substantially uniform sub-diastolic pressure field to the digit of the patient.
Clause 9: The optical digit probe of clause 8, wherein the pressure device is configured to facilitate unloading of arterial wall tension.
Clause 10: The optical digit probe of clause 8 or clause 9, wherein the pressure device is configured to mitigate distal venous pooling or distention to avoid induction of venoarterial-mediated vasoconstriction.
Clause 11: An optical digit probe for noninvasive measurement of at least one physiological parameter of a patient, the optical digit probe comprising: a digit housing portion comprising an external digit housing surface and an internal digit housing surface, wherein a proximal end of the digit housing portion is adapted for facilitating insertion of a digit of the patient into the digit housing portion; a skin securement portion configured to releasably couple the probe to a skin surface of the patient; an attachment portion extending between the external digit housing surface of the digit housing portion and the skin securement portion; and at least one optical sensor disposed in the digit housing portion of the probe configured to be positioned proximate to the digit of the patient to provide optical signals reflecting the at least one physiological parameter of the patient.
Clause 12: The optical digit probe of clause 11, wherein the attachment portion is elongated having a narrow distal end, a narrow proximal end, and longitudinal sides extending therebetween.
Clause 13: The optical digit probe of clause 12, wherein a longitudinal axis of the elongated attachment portion is substantially parallel to a longitudinal axis of the digit housing portion.
Clause 14: The optical digit probe of clause 11, wherein the attachment portion extends over the open proximal end of the digit housing portion.
Clause 15: The optical digit probe of any of clauses 11-14, wherein the digit housing portion, the skin securement portion, and the attachment portion are integrally formed by a single molding process.
Clause 16: The optical digit probe of any of clauses 11-15, wherein the attachment portion comprises an inwardly facing surface and an outwardly facing surface, and wherein the inwardly facing surface is adhered to the external digit housing surface of the digit housing portion.
Clause 17: The optical digit probe of clause 16, wherein the inwardly facing surface is permanently and immovably fixed to the external digit housing surface of the digit housing portion.
Clause 18: The optical digit probe of any of clauses 11-17, wherein a longitudinal axis of the skin securement portion is substantially transverse or angled relative to a longitudinal axis of the attachment portion.
Clause 19: The optical digit probe of any of clauses 11-18, wherein the optical digit probe is configured for non-invasive measurement of arterial vasomotor activity through the digit of the patient.
Clause 20: The optical digit probe of any of clauses 11-19, wherein the optical signals provided by the at least one optical sensor reflect an arterial pulse waveform of the patient.
Clause 21: The optical digit probe of any of clauses 11-20, wherein the digit comprises at least one of a little finger, a ring finger, a middle finger, or an index finger of the patient.
Clause 22: The optical digit probe of any of clauses 11-21, wherein the digit housing comprises a pressure device configured to apply a substantially uniform sub-diastolic pressure field to the digit of the patient.
Clause 23: The optical digit probe of clause 22, wherein the pressure device is configured to facilitate unloading of arterial wall tension.
Clause 24: The optical digit probe of clause 22 or clause 23, wherein the pressure device is configured to mitigate distal venous pooling or distention to avoid induction of venoarterial-mediated vasoconstriction.
Clause 25: An optical digit probe for noninvasive measurement of at least one physiological parameter of a patient, the optical digit probe comprising: a digit housing comprising an external digit housing surface and an internal digit housing surface, wherein a proximal end of the digit housing is adapted for facilitating insertion of a digit of the patient into the digit housing; an attachment device removably connected to the external digit housing surface of the digit housing comprising a skin securement portion extending from the digit housing configured to releasably couple the attachment device to a skin surface of the patient; and at least one optical sensor disposed in the digit housing configured to be positioned proximate to the digit of the patient to provide optical signals reflecting the at least one physiological parameter of the patient.
Clause 26: The optical digit probe of clause 25, wherein the skin securement portion extends over the proximal end of the digit housing.
Clause 27: The optical digit probe of clause 25 or clause 26, wherein the attachment device is configured to snap fit over the proximal end of the digit housing.
Clause 28: The optical digit probe of any of clauses 25-27, wherein the optical digit probe is configured for non-invasive measurement of arterial vasomotor activity through the digit of the patient.
Clause 29: The optical digit probe of any of clauses 25-28, wherein the optical signals provided by the at least one optical sensor reflect an arterial pulse waveform of the patient.
Clause 30: The optical digit probe of any of clauses 25-29, wherein the digit housing comprises a pressure device configured to apply a substantially uniform sub-diastolic pressure field to the digit of the patient.
Clause 31: The optical digit probe of clause 30, wherein the pressure device is configured to facilitate unloading of arterial wall tension.
Clause 32: The optical digit probe of clause 30 or clause 31, wherein the pressure device is configured to mitigate distal venous pooling or distention to avoid induction of venoarterial-mediated vasoconstriction.
Clause 33: An optical digit probe for noninvasive measurement of at least one physiological parameter of a patient, the optical digit probe comprising: a digit housing comprising an external digit housing surface and an internal digit housing surface, wherein a proximal end of the digit housing is adapted for facilitating insertion of at least portions of a first portion and a second portion of a digit of the patient into the digit housing; an attachment device comprising: a housing fastening portion connected to the external digit housing surface of the digit housing, and a skin securement portion configured to releasably couple the attachment device to a skin surface about a third portion of the digit of the patient; and at least one optical sensor disposed in the digit housing configured to be positioned proximate to the digit of the patient to provide optical signals reflecting the at least one physiological parameter of the patient.
Clause 34: The optical digit probe of clause 33, wherein the first portion comprises a distal portion of the digit, the second portion comprises a middle portion of the digit, and the third portion comprises a proximal portion of the digit extending from a palm of the patient.
Clause 35: The optical digit probe of clause 33 or clause 34, wherein the first portion comprises a portion of the digit about a distal phalanges of the patient, the second portion comprises a portion of the digit about a middle phalanges of the patient, and the third portion comprises a portion of the digit about a proximal phalanges of the patient.
Clause 36: The optical digit probe of any of clauses 33-35, wherein the digit housing is adapted for facilitating insertion of about a distal-most two-thirds (⅔) of the digit into the digit housing.
Clause 37: The optical digit probe of any of clauses 33-36, wherein the skin securement portion is configured to at least partially circumferentially enclose the third portion of the digit of the patient.
Clause 38. The optical digit probe of any of clauses 33-37, wherein the skin securement portion is configured to completely circumferentially enclose the third portion of the digit of the patient.
Clause 39: The optical digit probe of any of clauses 33-38, wherein the optical digit probe is configured for non-invasive measurement of arterial vasomotor activity through the digit of the patient.
Clause 40: The optical digit probe of any of clauses 33-39, wherein the optical signals provided by the at least one optical sensor reflect an arterial pulse waveform of the patient.
Clause 41: The optical digit probe of any of clauses 33-40, wherein the digit housing comprises a pressure device configured to apply a substantially uniform sub-diastolic pressure field to the digit of the patient.
Clause 42: The optical digit probe of clause 41, wherein the pressure device is configured to facilitate unloading of arterial wall tension.
Clause 43: The optical digit probe of clause 41 or clause 42, wherein the pressure device is configured to mitigate distal venous pooling or distention to avoid induction of venoarterial-mediated vasoconstriction.
Clause 44: An optical digit probe for noninvasive measurement of at least one physiological parameter of a patient, the optical digit probe comprising: a digit housing comprising an external digit housing surface and an internal digit housing surface, wherein a proximal end of the digit housing is adapted for facilitating insertion of a digit of the patient into the digit housing; an attachment device comprising: a housing fastening portion connected to the external digit housing surface of the digit housing, and an securement portion configured to be worn on a wrist, forearm, and/or arm of the patient for releasably coupling the attachment device to the patient, and at least one optical sensor disposed in the digit housing configured to be positioned proximate to the digit of the patient to provide optical signals reflecting the at least one physiological parameter of the patient.
Clause 45: The optical digit probe of clause 44, wherein the securement portion comprises an adhesive layer comprising a hydrogel or a reusable adhesive for adhering to the wrist, forearm, and/or arm of the patient.
Clause 46: The optical digit probe of clause 44 or clause 45, wherein the housing fastening portion and the securement portion form at least one strap comprising a first end and a second end, each of which are connected to the external digit housing surface, and an elongated member between the first end and the second end configured to be wrapped over a palm and around at least one of the wrist, forearm, and/or arm of the patient.
Clause 47: The optical digit probe of any of clauses 44-46, wherein the housing fastening portion and the securement portion form a first strap and a second strap, each strap comprising a first end and a second end, each of which are connected to the external digit housing surface, and an elongated member between the first end and the second end configured to be wrapped over a palm and around at least one of the wrist, forearm, and/or arm of the patient.
Clause 48: The optical digit probe of clause 47, wherein the first strap and the second strap are configured to cross over the palm of the patient.
Clause 49: The optical digit probe of any of clauses 44-48, wherein the attachment device further comprises a tether portion extending between the housing fastening portion and the securement portion.
Clause 50: The optical digit probe of any of clauses 44-49, wherein the optical digit probe is configured for non-invasive measurement of arterial vasomotor activity through the digit of the patient.
Clause 51: The optical digit probe of any of clauses 44-50, wherein the optical signals provided by the at least one optical sensor reflect an arterial pulse waveform of the patient.
Clause 52: The optical digit probe of any of clauses 44-51, wherein the attachment device comprises a longitudinal attachment device having a longitudinal axis corresponding to an insertion direction of the digit of the patient into the digit housing.
Clause 53: The optical digit probe of clause 52, wherein the housing fastening portion is on a distal end of the attachment device and the skin securement portion is on a proximal end of the attachment device, the attachment device further comprising a middle portion between the housing fastening portion and the skin securement portion.
Clause 54: The optical digit probe of clause 52 or clause 53, wherein the longitudinal attachment device is configured to be permanently fastened to the digit housing.
Clause 55: The optical digit probe of any of clauses 52-54, wherein the longitudinal attachment device is configured to be releasably fastened to the digit housing.
Clause 56: The optical digit probe of any of clauses 44-55, wherein the digit housing comprises a pressure device configured to apply a substantially uniform sub-diastolic pressure field to the digit of the patient.
Clause 57: The optical digit probe of clause 56, wherein the pressure device is configured to facilitate unloading of arterial wall tension.
Clause 58: The optical digit probe of clause 56 or clause 57, wherein the pressure device is configured to mitigate distal venous pooling or distention to avoid induction of venoarterial-mediated vasoconstriction.
Clause 59: An optical digit probe for noninvasive measurement of at least one physiological parameter of a patient, the optical digit probe comprising: a digit housing comprising an external digit housing surface and an internal digit housing surface, wherein a proximal end of the digit housing is adapted for facilitating insertion of a digit of the patient into the digit housing; an attachment device connected to the external digit housing surface of the digit housing comprising at least one anchor portion configured to be secured to at least one digit of the patient, the at least one digit being different from the digit inserted into the digit housing, thereby removably coupling the attachment device to the patient; and at least one optical sensor disposed in the digit housing configured to be positioned proximate to the digit of the patient to provide optical signals reflecting the at least one physiological parameter of the patient.
Clause 60: The optical digit probe of clause 59, wherein the at least one anchor portion is configured to be secured to the at least one digit of the patient that is adjacent to the digit inserted into the digit housing.
Clause 61: The optical digit probe of clause 59 or clause 60, wherein the digit housing is configured to be inserted onto a middle finger of the patient, and the at least one anchor portion of the attachment device is configured to be secured to an index finger and/or a ring finger of the patient.
Clause 62: The optical digit probe of any of clauses 59-61, wherein the at least one anchor portion comprises an annular member configured to receive the at least one digit adjacent to the digit inserted into the digit housing.
Clause 63: The optical digit probe of clause 62, wherein the annular member comprises at least one slot so that the annular member can expand for use with patients with larger digits.
Clause 64: The optical digit probe of any of clauses 59-63, wherein the optical digit probe is configured for non-invasive measurement of arterial vasomotor activity through the digit of the patient.
Clause 65: The optical digit probe of any of clauses 59-64, wherein the optical signals provided by the at least one optical sensor reflect an arterial pulse waveform of the patient.
Clause 66: The optical digit probe of any of clauses 59-65, wherein the digit housing comprises a pressure device configured to apply a substantially uniform sub-diastolic pressure field to the digit of the patient.
Clause 67: The optical digit probe of clause 66, wherein the pressure device is configured to facilitate unloading of arterial wall tension.
Clause 68: The optical digit probe of clause 66 or clause 67, wherein the pressure device is configured to mitigate distal venous pooling or distention to avoid induction of venoarterial-mediated vasoconstriction.
Clause 69: An optical digit probe for noninvasive measurement of at least one physiological parameter associated with a patient, the optical digit probe comprising: a housing comprising an open proximal end configured to receive a digit of the patient; a skin securement portion configured to releasably couple the housing to the digit, and resist movement of the digit out of the housing; and at least one optical sensor mounted within the housing and configured to be positioned proximate the digit of the patient to detect signals associated with the at least one physiological parameter.
Clause 70: The optical digit probe of any preceding clause, wherein the at least one optical sensor comprises an LED configured to project light of a predetermined wavelength on the patient, and at least one photodiode configured to receive light reflected from or transmitted through the patient.
Clause 71: The optical digit probe of clause 70, wherein the light projected by the LED is projected to the digit of the patient, and the light received by the at least one photodiode is at least one of reflected from the digit or transmitted through the digit for monitoring arterial tone associated with the digit.
Clause 72: The optical digit probe of any one of clauses 69-71, wherein the skin securement portion is configured to detachably couple to the housing.
Clause 73: The optical digit probe of any one of clauses 69-72, wherein the resistance by the skin securement portion to movement of the digit out of the housing is greater than resistance by the skin securement portion to movement of the digit into the digit housing.
Clause 74: The optical digit probe of any one of clauses 69-73, wherein the optical digit probe comprises a longitudinal axis, and the skin securement portion comprises a plurality of flexible members arranged as flaps in a transverse plane about a center, and wherein in an assembled configuration in which the skin securement portion is detachably coupled to the housing, the transverse plane is intersected by the longitudinal axis.
Clause 75: The optical digit probe of clause 74, wherein in the assembled configuration, each of the plurality of flexible members, when in a relaxed state, extends from a respective outer base portion toward the center.
Clause 76: The optical digit probe of clause 74 or clause 75, wherein in the assembled configuration, the transverse plane is perpendicular to the longitudinal axis of the optical digit probe.
Clause 77: The optical digit probe of clause 74, wherein in the assembled configuration, one or more of the flexible members comprises an outer base portion and an inner tip portion, and in a relaxed state extends along a radial axis in the transverse plane.
Clause 78: The optical digit probe of clause 77, wherein the one or more of the flexible members is configured to taper between the outer base portion and the inner tip portion.
Clause 79: The optical digit probe of clause 74, wherein one or more of the flexible members comprises a tapered shape which tapers towards a tip portion proximate the center.
Clause 80: The optical digit probe of clause 74, wherein one or more of the flexible members comprises a convex shape extending toward the center.
Clause 81: The optical digit probe of any one of clauses 74-80, wherein the plurality of flexible members are configured to bend and contact the digit of the patient along the digit when the digit is distally inserted into the open proximal end of the housing.
Clause 82: The optical digit probe of any one of clauses 74-81, wherein the skin securement portion defines a plurality of slots bordering the plurality of flexible members.
Clause 83: The optical digit probe of clause 80, wherein at least one of the plurality of flexible members is bordered by a respective pair of adjacent slots of the plurality of slots.
Clause 84: The optical digit probe of clause 80, wherein the skin securement portion defines a central hole in communication with the plurality of slots.
Clause 85: The optical digit probe of any one of clauses 74-84, wherein the skin securement portion defines an opening proximate a palmar facing region of the skin securement portion and configured to receive a palmar region of a proximal phalange of the digit of the patient.
Clause 86: The optical digit probe of any one of clauses 74-84, wherein the plurality of flexible members are arranged to form an opening at or proximate the palmar facing region of the skin securement portion and configured to receive a palmar region of a proximal phalange of the digit of the patient.
Clause 87: The optical digit probe of clause 85, wherein the opening is wedge-shaped.
Clause 88: The optical digit probe of clause 69, further comprising: a housing fastening portion configured to detachably couple the skin securement portion to the housing, wherein the housing of the optical digit probe comprises a first housing portion which houses the at least one optical sensor, and a second housing portion which comprises the open proximal end, and wherein the housing fastening portion comprises an engagement portion configured to receive the first housing portion and rotatably fix the housing fastening portion relative to the first housing portion.
Clause 89: The optical probe of clause 88, wherein the housing fastening portion comprises a body portion defining a channel configured to receive the second housing portion of the optical probe.
Clause 90: The optical digit probe of clause 69, wherein the skin securement portion comprises one or more grip members operatively disposed inside the housing and configured to grip the digit of the patient.
Clause 91: The optical digit probe of clause 90, wherein the one or more grip members comprise a pair of grip members on opposite sides of the housing inside the optical digit probe.
Clause 92: The optical digit probe of clause 90, wherein the one or more grip members comprises a membrane having a plurality of protrusions and extending at least one of circumferentially around or longitudinally along the housing inside the optical digit probe.
Clause 93: The optical digit probe of clause 92, wherein the membrane is made of a synthetic rubber and the plurality of protrusions are configured to grip the digit.
Clause 94: The optical probe of clause 90, wherein the housing comprises a pressure device configured to apply a substantially uniform pressure field to the digit of the patient.
Clause 95: The optical digit probe of clause 94, wherein the one or more grip members exert, in the presence of the uniform pressure field, a compressive force on the digit and provide resistance to movement of the digit out of the housing.
Clause 96: An attachment device for releasably coupling a digit of a patient to an optical digit probe configured for noninvasive measurement of at least one physiological parameter associated with the patient, the optical digit probe having a first housing portion which houses an optical sensor assembly, and a second housing portion which comprises an open proximal end, the attachment device comprising: a housing fastening portion configured to detachably couple to at least one of the first housing portion or the second housing portion; and a skin securement portion configured to detachably couple to the housing fastening portion, allow distal translation of the digit of the patient into the optical digit probe, and resist proximal translation of the digit of the patient out of the optical digit probe.
Clause 97: The attachment device of clause 96, wherein the housing fastening portion comprises an engagement portion configured to receive and detachably couple to the second housing portion to rotatably fix the housing fastening portion relative to the optical sensor assembly.
Clause 98: The attachment device of clause 96 or clause 97, wherein the housing fastening portion comprises a body portion defining a channel configured to receive the second portion of the housing of the optical digit probe.
Clause 99: The attachment device of any one of clauses 96-98, wherein the attachment device comprises a longitudinal axis, the skin securement portion comprises a plurality of flexible members, and in an assembled configuration in which the skin securement portion is detachably coupled to the housing fastening portion, the plurality of flexible members extend in a direction transverse to the longitudinal axis of the attachment device.
Clause 100: An attachment device for releasably coupling a digit of a patient to an optical digit probe configured for noninvasive measurement of at least one physiological parameter associated with the patient, the optical digit probe having a first housing portion which houses an optical sensor assembly, and a second housing portion which comprises an open proximal end, the attachment device comprising: a skin securement portion configured to detachably couple to the housing of the optical digit probe and the digit of the patient, allow distal translation of the digit into the optical digit probe, and resist proximal translation of the digit out of the optical digit probe.
Clause 101: The attachment device of clause 100, wherein the skin securement portion comprises a plurality of flexible members configured to, when the attachment device is mounted to the to the optical digit probe (i) in a relaxed state, extend in a direction transverse to a longitudinal axis of the optical digit probe, and (ii) bend and contact the digit of the patient along the digit when the digit is inserted into the optical digit probe.
Clause 102: The attachment device of clause 101, wherein when the attachment device is mounted to the optical digit probe, the plurality of flexible members, when in a relaxed state, extend perpendicular to the longitudinal axis of the optical digit probe.
Clause 103: The optical digit probe of clause 101 or clause 102, wherein the plurality of flexible members are configured to bend and contact the digit of the patient along the digit when the digit is inserted into the optical digit probe.
Clause 104: The optical digit probe of any one of clauses 101-103, wherein each flexible member has an outer base portion and an inner tip portion, and tapers between the outer base portion and the inner tip portion.
Clause 105: The optical digit probe of clause 100, wherein the skin securement portion defines a plurality of slots bordering the plurality of flexible members, and a central hole in communication with the plurality of slots.

### BRIEF DESCRIPTION OF THE DRAWINGS

The terms Fig., Figs., Figure, and Figures are used interchangeably in the specification to refer to the corresponding figures in the drawings.

Various aspects of the disclosure are discussed below with reference to the accompanying figures, which are not intended to be drawn to scale. The figures are included to provide an illustration and a further understanding of various examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. A quantity of each component in a particular figure is an example only and other quantities of each, or any, component could be used.

FIG. 1 is a perspective view of an optical digit probe according to an aspect of the present disclosure;

FIG. 2 is a perspective view of another example of an optical digit probe according to an aspect of the present disclosure;

FIG. 3A is a perspective view of an optical digit probe including an attachment device, according to an aspect of the present disclosure;

FIG. 3B is another perspective view of the optical digit probe of FIG. 3A;

FIG. 3C is a drawing showing the optical digit probe of FIG. 3A inserted over a middle finger of a patient, according to an aspect of the present disclosure;

FIG. 3D is a front view of another example of an optical digit probe including an attachment device, according to an aspect of the present disclosure;

FIG. 3E is a drawing showing the optical digit probe of FIG. 3A inserted over a little finger of a patient;

FIG. 3F is a drawing showing the optical digit probe of FIG. 3A inserted over a ring finger of a patient;

FIG. 3G is a drawing showing the optical digit probe of FIG. 3A inserted over an index or pointer finger of a patient;

FIG. 3H is a perspective view of another example of an optical digit probe including the attachment device of FIG. 3A connected to a clip-type digit probe, according to an aspect of the present disclosure;

FIG. 4A is an exploded view of an attachment device configured to be mounted to an optical digit probe, according to an aspect of the present disclosure;

FIG. 4B is an exploded view showing the attachment device of FIG. 4A separated from a housing of an optical digit probe, according to an aspect of the present disclosure;

FIG. 4C is a perspective view of the optical digit probe and attachment device of FIG. 4B;

FIG. 4D is another perspective view of the optical digit probe and attachment device of FIG. 4B;

FIG. 4E is a drawing showing the optical digit probe and attachment device of FIG. 4B inserted onto a middle finger of a patient, according to an aspect of the present disclosure;

FIG. 4F is a side perspective view of another example of an optical digit probe in an assembled configuration, in accordance with the present disclosure;

FIG. 4G is another side perspective view of the optical digit probe of FIG. 4F in the assembled configuration;

FIG. 4H is a front perspective view of the optical digit probe of FIG. 4F, with a finger of a patient extending therethrough, in accordance with the present disclosure;

FIG. 4I is a side perspective view of the housing fastening portion of the attachment device of the optical digit probe of FIG. 4F;

FIG. 4J is a top view of a plurality of removable skin securement portions attachable to the housing fastening portion of FIG. 4F, in accordance with the present disclosure;

FIG. 4K is a top perspective view of the removable skin securement portion and the housing fastening portion of FIG. 4F;

FIG. 4L is a rear perspective view of the removable skin securement portion and the housing fastening portion of FIG. 4F;

FIG. 4M is a rear perspective view of the housing fastening portion and skin securement portion of the attachment device of FIG. 4F, showing a finger of a patient extending therethrough, in accordance with the inventive disclosure;

FIG. 4N is a rear perspective view of a skin securement portion prior to attachment to the housing fastening portion, showing a film being peeled back to expose adhesive on a rear surface of the skin securement portion, in accordance with the present disclosure;

FIG. 4O is a front view of a plurality of alternative removable skin securement portions attachable to the housing fastening portion of FIG. 4F, in accordance with the present disclosure;

FIG. 4P is a top perspective view of one of the alternative removable skin securement portions of FIG. 4O assembled with the housing fastening portion, showing a finger of a patient extending therethrough;

FIG. 4Q is a side perspective view of the optical digit probe and housing fastening portion of FIG. 4F, with one of the alternative removable skin securement portions of FIG. 4O assembled therewith, in accordance with the present disclosure;

FIG. 4R is a side perspective view of an optical digit probe with a skin securement portion attached directly thereto without a housing fastening portion, in accordance with the present disclosure;

FIG. 4S is a side perspective view of an optical digit probe with a skin securement portion extending radially within an open proximal end thereof, in accordance with the present disclosure;

FIG. 4T is a front view of alternatively shaped tapered regions of a skin securement portion, in accordance with the present disclosure;

FIG. 4U is a schematic diagram of a hand showing proximal, distal, palmer, and dorsal directions;

FIG. 4V is a front cutaway view of another example of an optical digit probe having a skin securement portion fully disposed inside the probe on one side thereof, in accordance with the present disclosure;

FIG. 4W is a front cutaway view of another example of an optical digit probe having a skin securement portion fully disposed inside the probe on opposite sides thereof, in accordance with the present disclosure;

FIG. 4X is a front cutaway view of yet another example of an optical digit probe having a skin securement portion fully disposed inside the probe on opposite sides thereof, and made from an alternative material, in accordance with the present disclosure;

FIG. 4Y is a top perspective view of a membrane with grip features, in accordance with the present disclosure;

FIG. 5A is a perspective view of another example of an optical digit probe showing an attachment device of the probe in an in-use configuration, according to an aspect of the present disclosure;

FIG. 5B is another perspective view of the optical digit probe of FIG. 5A with the attachment device in the in-use configuration;

FIG. 5C is an exploded perspective view of the optical digit probe of FIG. 5A with the attachment device in a pre-use configuration;

FIG. 5D is a perspective view of the optical digit probe of FIG. 5A with the attachment device in the pre-use configuration;

FIG. 5E is a drawing showing the optical digit probe of FIG. 5A inserted onto a middle finger of a patient, according to an aspect of the present disclosure;

FIG. 5F is a drawing showing the optical digit probe of FIG. 5A inserted onto a little finger of a patient;

FIG. 5G is a drawing showing the optical digit probe of FIG. 5A inserted onto a ring finger of a patient;

FIG. 5H is a drawing showing the optical digit probe of FIG. 5A inserted onto a pointer or index finger of a patient;

FIG. 6A is a perspective view of another example of an optical digit probe, according to an aspect of the present disclosure;

FIG. 6B is another perspective view of the optical digit probe of FIG. 6A;

FIG. 6C is a drawing showing the optical digit probe of FIG. 6A inserted onto a finger of a patient, according to an aspect of the present disclosure;

FIG. 6D is a perspective view of another example of an optical digit probe including the attachment device of FIG. 6A connected to a clip-type digit probe, according to an aspect of the present disclosure;

FIG. 7 is a perspective view of another example of an optical digit probe, according to an aspect of the present disclosure;

FIG. 8 is a perspective view of another example of an optical digit probe, according to an aspect of the present disclosure;

FIG. 9A is a perspective view of another example of an optical digit probe, according to an aspect of the present disclosure;

FIG. 9B is another perspective view of the optical digit probe of FIG. 9A;

FIG. 9C is a drawing showing the optical digit probe of FIG. 9A inserted onto a middle finger of a patient, according to an aspect of the present disclosure;

FIG. 10A is a perspective view of another example of an optical digit probe including an attachment device worn about a wrist of a patient, according to an aspect of the present disclosure;

FIG. 10B is a drawing showing the optical digit probe of FIG. 10A inserted onto a middle finger of a patient with the attachment device around a wrist of the patient, according to an aspect of the present disclosure;

FIG. 10C is a front view of another example of an optical digit probe including an attachment device, according to an aspect of the present disclosure;

FIG. 10D is a perspective view of the optical digit probe and attachment device of FIG. 10C worn about a wrist of a patient;

FIG. 10E is a drawing showing the optical digit probe of FIG. 10D inserted onto a middle finger of a patient with the attachment device around a wrist of the patient, according to an aspect of the present disclosure;

FIG. 10F is a drawing showing the optical digit probe of FIG. 10D inserted onto a ring finger of a patient with the attachment device around a wrist of the patient;

FIG. 10G is a drawing showing the optical digit probe of FIG. 10D inserted onto a pointer or index finger of a patient with the attachment device around a wrist of the patient;

FIG. 10H is a perspective view of another example of an optical digit probe including the attachment device of FIG. 10C connected to a clip-type digit probe, according to an aspect of the present disclosure;

FIG. 11A is an exploded perspective view of another example of an optical digit probe including an attachment device configured to be secured to an adjacent digit of a patient, according to an aspect of the present disclosure;

FIG. 11B is a perspective view of the optical digit probe of FIG. 11A;

FIG. 11C is a drawing showing the optical digit probe of FIG. 11A inserted onto a middle finger of the patient with the attachment device secured to an adjacent finger, according to an aspect of the present disclosure;

FIG. 11D is a drawing showing the optical digit probe of FIG. 11A inserted onto a pointer or index finger of the patient with the attachment device secured to an adjacent finger;

FIG. 11E is a drawing showing the optical digit probe of FIG. 11A inserted onto a ring finger of the patient with the attachment device secured to an adjacent finger;

FIG. 11F is a perspective view of another example of an optical digit probe including the attachment device of FIG. 11A connected to a clip-type digit probe, according to an aspect of the present disclosure;

FIG. 12A is a perspective view of another example of an optical digit probe including an attachment device configured to be secured to two adjacent digits of a patient, according to an aspect of the present disclosure;

FIG. 12B is a drawing showing the optical digit probe of FIG. 12A inserted onto a finger of the patient with the attachment device secured to two adjacent fingers of the patient, according to an aspect of the present disclosure;

FIG. 13A is a schematic drawing of a medical device including an optical digit probe and wearable monitor, according to an aspect of the present disclosure;

FIG. 13B is a schematic drawing of components of the medical device of FIG. 13A;

FIG. 13C is a schematic drawing showing the medical device worn by a patient;

FIG. 14 is a schematic drawing showing a finger of the patient inserted in the optical digit probe of FIG. 13A, according to an aspect of the present disclosure;

FIGS. 15A-15C are schematic drawings of a physiological monitoring system including a medical device comprising an optical digit probe and a wearable monitor and a remote server in electronic communication with the medical device, according to aspects of the present disclosure; and

FIGS. 16A-16F are user interface screens that can be displayed on a mobile or computer device guiding a patient in performing at at-home medical test, according to aspects of the present disclosure.

### DESCRIPTION

Implementations are described herein to provide for improved optical digit probes for use with wearable medical devices. For example, such wearable medical devices can be used to monitor a variety of patient physiological signals or parameters over the duration of a study period. Medical optical digit probes can be used in healthcare settings to measure, e.g., a level of oxygen saturation in a patient's blood, a patient's arterial pulse, and/or respiratory parameters of the patient. In examples, the digit probe as described herein can be attached to a patient's finger, toe, or earlobe (or other extremity of the patient's body). For example, in some implementations, such a probe can use light to measure the absorption of oxygen in the blood. The probe can contain both a light-emitting diode and a photodetector, which work together to measure the level of oxygen saturation in the patient's blood by shining a beam of light through the tissue and detecting the amount of light that is absorbed. The device can calculate the oxygen saturation level by comparing the ratio of absorbed to unabsorbed light. In certain scenarios, such optical digit probes can be used during surgeries or in critical care settings, where it is important to monitor a patient's oxygen levels to ensure they are receiving adequate oxygenation. In yet some example scenarios, optical digit probes as described herein can be used in monitoring SDB conditions in a patient, e.g., sleep apnea and/or related conditions.

In examples, an optical digit probe can include a tubular, cylindrical, or similar elongated housing sized so that a digit to be monitored can be inserted into an interior of the housing and an optical sensor for detecting signals representative of patient physiological parameters. The optical sensor can include a light (e.g., visible or infrared light) emitter and a receiver or detector for receiving light reflected by the digit of the patient. Other optical digit probes are configured as clip or clamp including portions that press against the digit being monitored. Such clip-type probes can include a housing formed from upper and lower elongated trays or bodies pivotally connected together at a distal end thereof. The digit to be monitored can be inserted between the elongated bodies or trays, and the elongated bodies or trays can be permitted to pivot towards each other, thereby securing the housing to the digit to be monitored.

Optical digit probes can be used for a variety of medical applications. For example, such probes can be used by a medical caregiver to obtain an instantaneous reading for oxygen saturation, blood pressure, and other patient physiological parameters. After an acceptable reading is obtained, the caregiver can remove the optical digit probe from the patient's digit. Optical digit probes can also be used for continuous long-term patient monitoring for a period of hours, or for an entire day, or an entire night. An optical digit probe can also be used outside or remote from a medical facility, such as for home and/or remote sleep apnea tests (e.g., HSATs) and/or home and/or remote sleep disordered breathing tests for diagnosing sleep apnea.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the disclosure.

As used herein, the singular form of “a”, “an”, and “the” include plural referents unless the context clearly dictates otherwise.

As used herein, the terms “right”, “left”, “top”, and derivatives thereof shall relate to aspects of the present disclosure as it is oriented in the drawing figures. However, it is to be understood that embodiments of the present disclosure can assume various alternative orientations and, accordingly, such terms are not to be considered as limiting. Also, it is to be understood that embodiments of the present disclosure can assume various alternative variations and stage sequences, except where expressly specified to the contrary. It is also to be understood that the specific devices and processes illustrated in the attached drawings, and described in the following specification, are provided as examples. Hence, specific dimensions and other physical characteristics related to the embodiments disclosed herein are not to be considered as limiting.

As used herein, including in the claims, “and” as used in a list of items prefaced by “at least one of” indicates a disjunctive list such that, for example, a list of “at least one of A, B, and C” means A or B or C or AB or AC or BC or ABC (i.e., A and B and C), or combinations with more than one feature (e.g., AA, AAB, ABBC, etc.). As used herein, including in the claims, unless otherwise stated, a statement that a function or operation is “based on” an item or condition means that the function or operation is based on the stated item or condition and may be based on one or more items and/or conditions in addition to the stated item or condition.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data, and does not actively transmit data to the second unit. As another example, a first unit can be in communication with a second unit if an intermediary unit processes data from one unit and transmits processed data to the second unit. It will be appreciated that numerous other arrangements are possible.

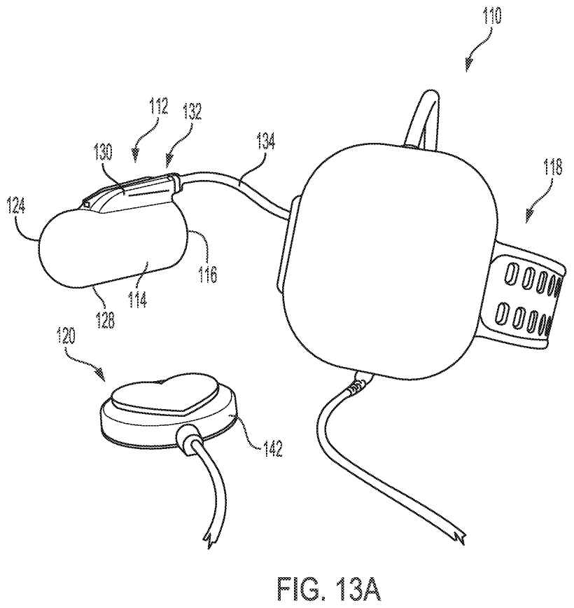

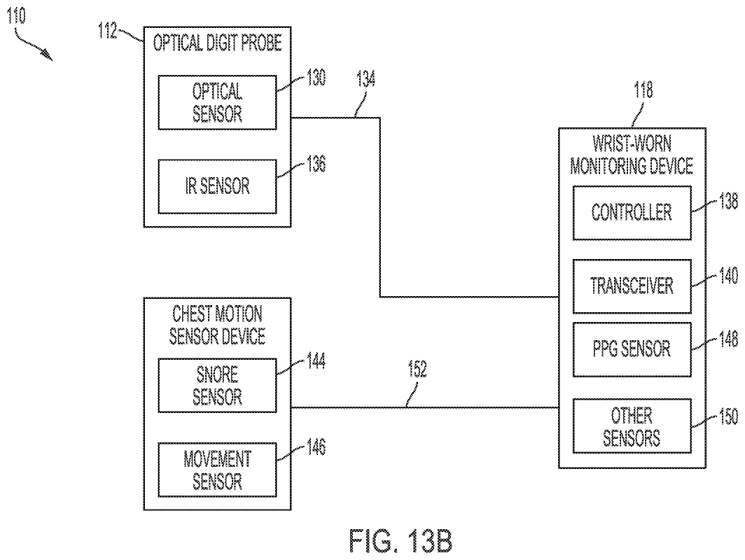

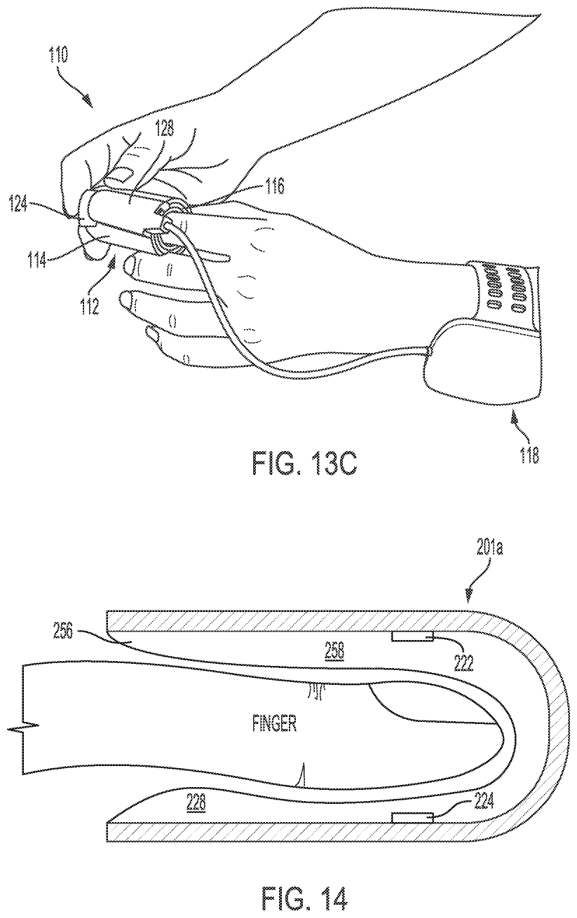

The present disclosure is directed to medical systems and devices 110 (shown in FIGS. 13A-15C) that can be used for patient monitoring to detect, record, and process data for one or more patient physiological parameters. In particular, the medical systems and devices 110 disclosed herein can be configured for short-term use (e.g., for a few minutes to about an hour, such as during a brief visit to a caregiver facility) and/or long-term and/or continuous use (e.g., in the context of both inpatient and outpatient monitoring. For example, the medical systems and devices 110 can be used by patients at home and/or remote from a medical facility. The medical systems and devices 110 disclosed herein are configured to be used and/or worn by a patient or wearer. The patient can be inpatient, e.g., a patient admitted to a medical facility, such as a hospital, rehabilitation center, a long term care center, a nursing facility, an assisted living facility, a long term memory care center, or other inpatient clinical facility. Additionally or alternatively, the patient can be outpatient, e.g., a patient that is visiting a healthcare facility, a caregiver, or other medical professional for a short duration, or remotely visiting via telemedicine facilities with the facility.

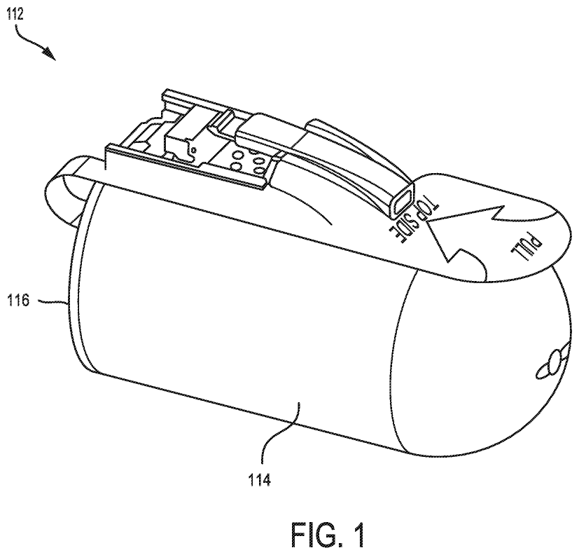

The medical systems and medical devices 110 disclosed herein can comprise an optical digit probe 112 (shown in FIGS. 1, 2, and 13A-15C), shown therein as a finger probe, for noninvasive measurement of a physiological parameter of a wearer or patient. In some examples, the optical digit probe 112 can be a component of an oximeter for obtaining oxygen saturation measurements for the patient. Physiological parameters that can be determined from data collected by the optical digit probe 112 can also include other parameters representative of respiratory function, such as parameter values for oxygen blood levels or oxygen update (e.g., VO₂max). Physiological parameters that can be determined from data collected by the optical digit probe 112 can also include cardiac or blood flow parameters, such as arterial pulse parameters, blood pressure parameters, or heart rate parameters. Physiological parameters can also include blood metrics, such as blood glucose level, glycemic index, insulin index, fat body composition, protein body composition, blood nutrient level (e.g., iron), blood sodium levels, and/or naturally-produced chemical compound level (e.g., lactic acid).

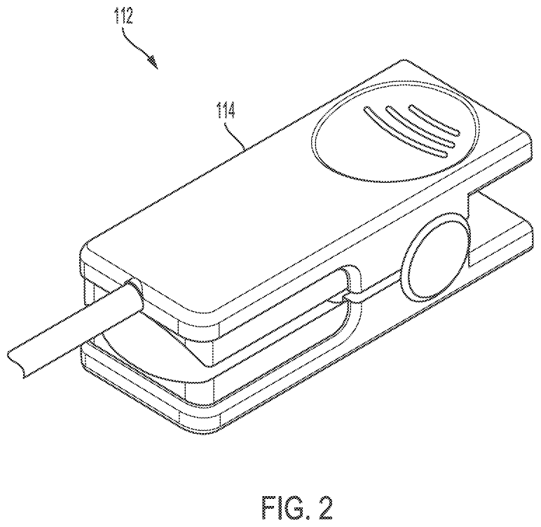

As shown in FIG. 1, an example optical digit probe 112 comprises a tubular, cylindrical, or similar elongated housing 114 including an open proximal end 116 sized so that a digit to be monitored can be inserted into an interior of the housing 114. As shown in FIG. 2, in other examples, an optical digit probe 112 can have a housing 114 comprising a clip formed from upper and lower elongated trays or bodies pivotally connected together at a distal end thereof. The digit to be monitored can be inserted between the elongated bodies or trays, and the elongated bodies or trays can be permitted to pivot towards each other, thereby securing the housing 114 to the digit to be monitored.

Patients using medical devices 110, such as the optical digit probes 112, often move around during a testing or monitoring activity. For example, the testing or monitoring activity may be a sleep study (e.g., in a sleep lab or a remote home-based sleep study) that can last for an overnight or a long study duration (e.g., between about 3-4 hours, about 6-8 hours, or about 9-12 hours). While examples described herein are in the context of long, continuous duration studies, it is understood that the systems, apparatuses, and methods described herein can be used for shorter duration applications as well. For example, the testing or monitoring activity may be an oxygen saturation measurement during a clinical visit, e.g., lasting for a short duration such as about 10 seconds to about 5 minutes, more particularly about 10 seconds to about 1 minute. The testing or monitoring activity may also be a longer clinical study for e.g., arterial pressure pulse changes, blood pressure monitoring, oxygen saturation changes, etc., during a clinical visit lasting, e.g., for 5 minutes to about an hour. For example, patients may change position, sit-up, stand, walk around, or perform other exercises and activities during monitoring or testing. While sleeping, patients may voluntarily or involuntarily change position or attempt to move, adjust, or reposition sheets, pillows, and other bedding materials. These voluntary or involuntary movements can modify a position or orientation of the optical digit probe 112 relative to the digit being monitored or can cause the optical digit probe 112 to fall off or detach from the patient, which can cause signal loss and/or a loss in signal fidelity for medical signals being detected by the optical digit probe 112. In order to avoid such signal loss, the optical digit probe 112 and/or other medical devices 110 can be secured to the patient in an improved manner so that the optical digit probe 112 does not change orientation or fall off even if, for example, the optical digit probe 112 catches on or attaches to sheets, bedding, the patient's clothes, or any other objects in proximity to the patient.

In order to ensure that the optical digit probe 112 remains securely attached to the patient for the duration of the study, the present disclosure is also directed to attachment devices for securing the optical digit probe 112, such as the probes 112 shown in FIGS. 1 and 2, in place on the digit of the patient. For example, the attachment devices disclosed herein can include various arrangements of straps, tethers, bands, sleeves, collars, and similar connectors for securing the optical digit probe 112 to a particular digit of the patient. The attachment device can be configured to hold the optical digit probe 112 in place on the digit of the patient during continuous, long-term use, thus preventing the optical digit probe 112 from becoming dislodged, disconnected, or from falling off of the digit of the patient at an inappropriate or unexpected time. Additionally or alternatively, the various attachment mechanisms described herein are configured to optimize the quality of the physiological data retrieved during the underlying clinical study. By ensuring proper fit and attachment to the relevant patient extremity, implementations described herein allow for greater reliance on clinical diagnosis that can be drawn from the resulting physiological data. Therefore, the attachment devices of the present disclosure can prevent loss or interruption of signal fidelity and/or medical data, which occurs when the optical digit probe 112 falls off of the digit of the patient. The attachment devices of the present disclosure can also ensure that data collected by sensors of the optical digit probe 112 is accurate, complete, and is not corrupted or modified by movement noise or changes in position of the optical digit probe 112 relative to the digit.

In some examples, the attachment device can be made from soft and flexible materials designed to tightly constrict against surfaces of the optical digit probe 112 and/or against the digit of the wearer, thereby forming a secure engagement between the optical digit probe 112 and the digit. For example, the attachment devices secured to the optical digit probe 112 can be partially or entirely formed from a thermoplastic elastomer, such as silicone, polypropylene, low-density polyethylene, synthetic rubber (e.g., polychloroprene), or natural rubber (e.g., isoprene). In other examples, portions of the attachment device can be formed from more rigid plastic materials, such as acrylonitrile butadiene styrene (ABS), polyester, polycarbonate, polypropylene, high-density polyethylene, or polyethylene terephthalate.

In some examples, the medical systems and devices 110 of the present disclosure can be used for diagnosis and monitoring of respiratory conditions of a patient. For example, an optical digit probe 112 can be attached to a digit of a patient to obtain an instantaneous pulse oximetry measurement for the patient. After the instantaneous measurement is obtained, the optical digit probe 112 can be removed or can remain in place on the digit of the patient for an expected duration providing continuous or periodic patient monitoring. In particular, the optical digit probe 112 may be intended to be worn by a patient for a long duration, such as a duration of at least several hours, overnight during a sleep study, or for a multi-night sleep study. In such long term use cases, the attachment device can be used to ensure that the optical digit probe 112 remains in place for the entirety of the study or monitoring duration.

As previously described, the optical digit probe 112 can comprise optical sensors for detecting signals representative of parameters including oxygen saturation, blood pressure, heart rate, and/or other respiratory and/or physiological parameters of the wearer. More specifically, in some examples, optical digit probes 112 of the present disclosure can be used as a pulse oximeter for non-invasive monitoring of a photoplethysmography (PPG) of a patient. Alternatively or in addition, sensors of the optical digit probe 112 can be configured to measure other cardiac or blood flow parameters, such as blood pressure or heart rate.

In some examples, the medical systems and devices 110 are configured to detect and measure respiratory and/or physiological parameters related to sleep apnea and/or disordered breathing. Sleep apnea is a common sleep disorder, which affects millions of people. With this condition, a person may have an interruption in his or her breathing while sleeping that occurs through repetitive pauses or apneic events. There are several types of sleep apnea, of which two prominent types include obstructive sleep apnea (OSA) and central sleep apnea (CSA). CSA can be characterized by pauses in breathing due to a lack of respiratory effort during sleep.

In CSA, the pauses in breathing throughout the night may be due to the lack of respiratory muscles activating or the brain failing to cause the respiratory muscles to activate. CSA can often be characterized as Cheyne-Stokes Respiration (CSR) in heart failure (HF) patients, and is a common comorbidity, affecting 30-50% of patients with reduced left ventricular ejection fraction (LVEF) and up to 18-30% of patients with preserved LVEF. Untreated CSA has shown to be independently associated with increased mortality and hospitalizations, especially in patients with heart failure. The symptoms of sleep apnea-fatigue, daytime somnolence, shortness of breath, and nocturnal dyspnea-often overlap with those of heart failure, making it difficult to evaluate the effect of cardiovascular therapies on patient symptoms while the sleep apnea remains untreated. Clinicians are commonly presented with complaints of fatigue and problems sleeping, which could be due to OSA or CSA. Understanding which patients to systematically screen and test for OSA and/or CSA can be challenging. In addition, heart failure clinicians need to be aware of treatment options for their patients as they may differ from the best options for other sleep apnea patients.

For these reasons, there is a need for medical devices and testing systems for at home or remote sleep studies, which allow for systematic screening of more patients than could be tested at specialized centers or facilities. The medical systems and devices 110 disclosed herein can be used for such home and/or remote sleep apnea tests (e.g., HSATs) and/or home and/or remote sleep disordered breathing (SDB) tests for diagnosing sleep apnea and/or for sleep stage (e.g., Rapid Eye Movement (REM), Light Sleep, Deep Sleep and Wake) identification for a patient or subject. Beneficially, such tests do not need to be performed at a sleep study center or facility. Instead, the tests can be performed at a patient's home allowing the patient to sleep in his or her own bed, meaning that falling asleep can be easier and more convenient than for sleep studies performed at specialized centers or facilities. Further, the attachment devices of the present disclosure can be used to prevent the optical digit probe 112 from becoming dislodged or from falling off of the digit of the patient, ensuring that data collected by the optical digit probe 112 during the home or remote sleep test is accurate, complete, and free from artefacts resulting from movement of the optical digit probe 112 relative to the digit being monitored.

Information collected by the optical digit probe 112 and other sensors of the medical device 110 can be used for providing various types of medical feedback and information for clinicians and for the patient. For example, the medical device 110 can be configured to generate indices representative of respiratory function and/or breathing quality of the wearer or patient based on signals detected by sensors of the optical digit probe 112. For example, the generated indices can include one or more of: a respiratory disturbance index; an apnea-hypopnea index; a central apnea-hypopnea index; and/or a percentage of total sleep time with Cheyne-Stokes Respiration pattern (% CSR). Information detected by the sensors of the optical digit probe 112 can also be analyzed to provide information representative of sleep staging identification. The respiratory indices and sleep staging information can be estimates determined from conventional values produced by polysomnography. The medical device 110 can also generate data representative of detected acoustic signals (e.g., acoustic decibel detection) used for monitoring snoring level and body position information representative of discrete states from motion signals from an accelerometer of a chest sensor of the medical device.

As described in further detail herein, in some examples, the medical systems and devices 110 of the present disclosure can also include monitoring or sensing devices separate from the optical digit probe 112, which can be coupled to other portions of a patient's body than the optical digit probe 112. For example, the medical systems and devices 110 disclosed herein can further comprise a wrist-worn monitor 118 (shown in FIGS. 13A-13C) or device in wired or wireless communication with the optical digit probe 112. The wrist-worn monitor 118 can include processing circuitry for receiving and processing data from the optical digit probe 112. In addition, the wrist-worn monitor 118 can include wireless communications circuitry and a processor coupled to the memory for storing recorded data and for wirelessly transmitting data from the optical digit probe 112 and/or wrist-worn monitor 118 to remote servers or computer devices. The medical system and device 110 can also include a chest motion sensor device 120 (shown in FIGS. 13A-13C) configured to be disposed on an upper chest area of the wearer or patient. The chest motion sensor device 120 can including a snoring microphone and an accelerometer. In some examples, the wrist-worn monitor 118 and/or the chest motion sensor device 120 can also include ECG electrodes and a body impedance sensor.

For convenience, this disclosure presents attachment mechanisms that are broadly presented as follows.

Example features that relate to attachment devices that are permanently attached to or an integral part of the optical probe housing.

Example features that relate to attachment devices that are releasably fastened to the optical probe housing.

Example features that relate to attachment devices configured to attach to the same digit (e.g., finger) as the digit probe is worn on.

Example features that relate to attachment devices configured to attach to a wrist of the arm corresponding to the finger on which the digit probe is worn.

Example features that relate to attachment devices configured to attach to an adjacent or neighboring digit (e.g., finger) to the finger on which the digit probe is worn.

An optical digit probe can include some or all of the example features described herein. For instance, in one implementation, an optical digit probe can include example features that relate to attachment devices that are permanently attached to or an integral part of the optical probe housing, and example features that relate to attachment devices configured to attach to the same digit (e.g., finger) as the digit probe is worn on. In an implementation, an optical digit probe can include example features that relate to attachment devices that are permanently attached to or an integral part of the optical probe housing, and example features that relate to attachment devices configured to attach to a wrist of the arm corresponding to the finger on which the digit probe is worn. In an implementation, an optical digit probe can include example features that relate to attachment devices that are permanently attached to or an integral part of the optical probe housing, and example features that relate to attachment devices configured to attach to an adjacent or neighboring digit (e.g., finger) to the finger on which the digit probe is worn.

For instance, in one implementation, an optical digit probe can include example features that relate to attachment devices that are releasably fastened to the optical probe housing, and example features that relate to attachment devices configured to attach to the same digit (e.g., finger) as the digit probe is worn on. In an implementation, an optical digit probe can include example features that relate to attachment devices that are releasably fastened to the optical probe housing, and example features that relate to attachment devices configured to attach to a wrist of the arm corresponding to the finger on which the digit probe is worn. In an implementation, an optical digit probe can include example features that relate to attachment devices that are releasably fastened to the optical probe housing, and example features that relate to attachment devices configured to attach to an adjacent or neighboring digit (e.g., finger) to the finger on which the digit probe is worn.

For instance, in one implementation, an optical digit probe can include example features that relate to attachment devices configured to attach to the same digit (e.g., finger) as the digit probe is worn on, and example features that relate to attachment devices configured to attach to a wrist of the arm corresponding to the finger on which the digit probe is worn. In an implementation, an optical digit probe can include example features that relate to attachment devices configured to attach to the same digit (e.g., finger) as the digit probe is worn on, and example features that relate to attachment devices configured to attach to an adjacent or neighboring digit (e.g., finger) to the finger on which the digit probe is worn.

For instance, in one implementation, an optical digit probe can include example features that relate to attachment devices configured to attach to the same digit (e.g., finger) as the digit probe is worn on, and example features that relate to attachment devices configured to attach to a wrist of the arm corresponding to the finger on which the digit probe is worn.

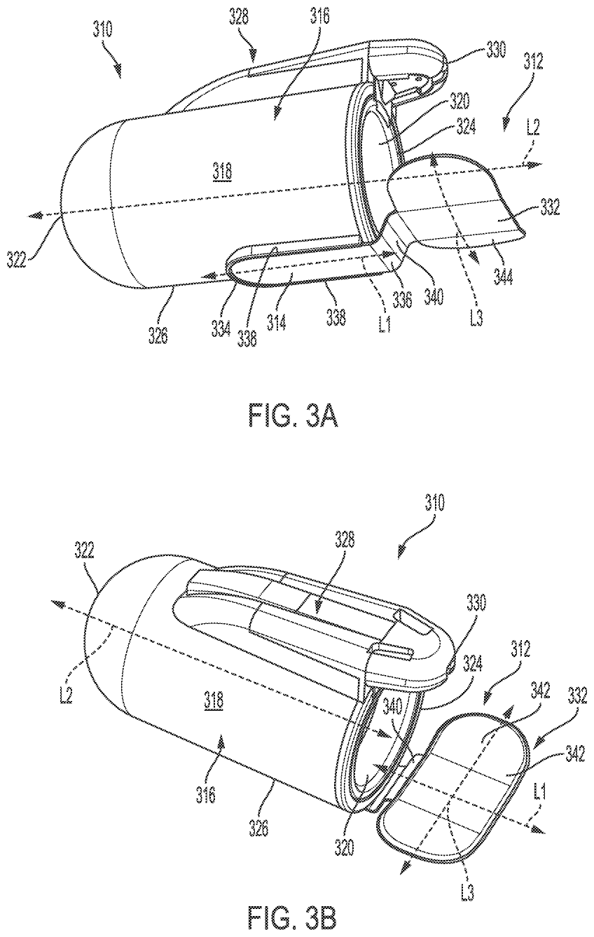

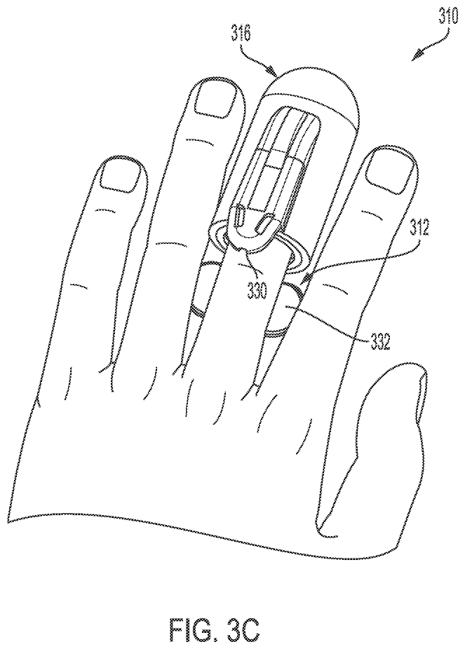

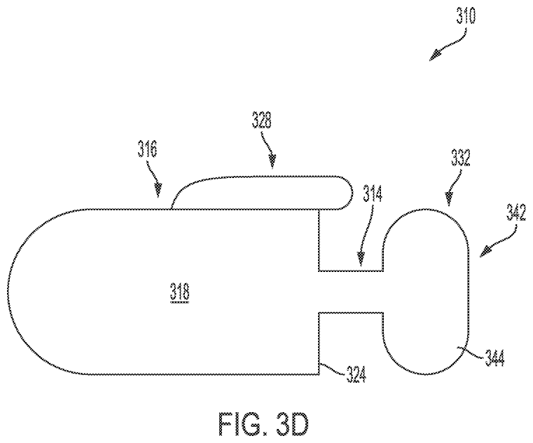

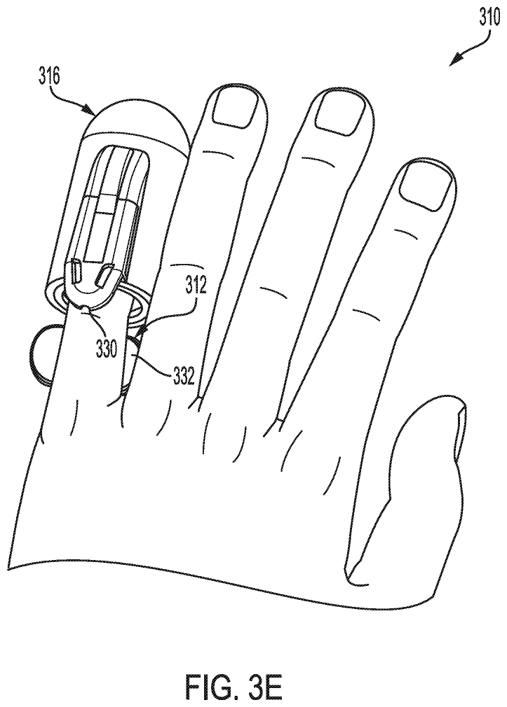

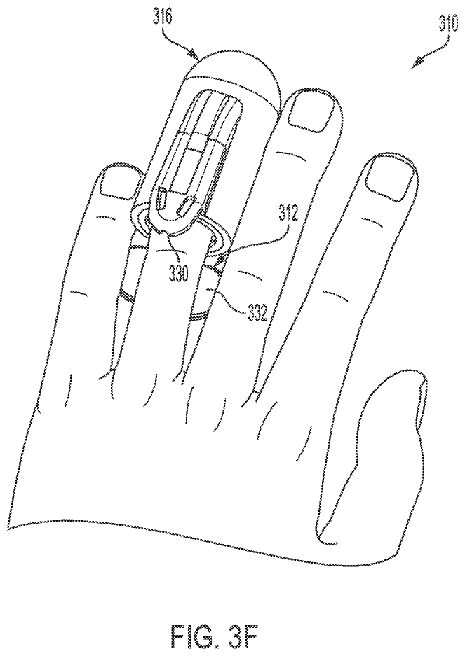

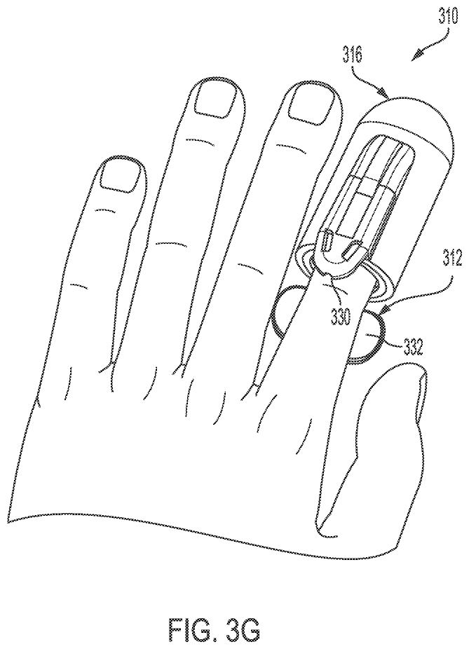

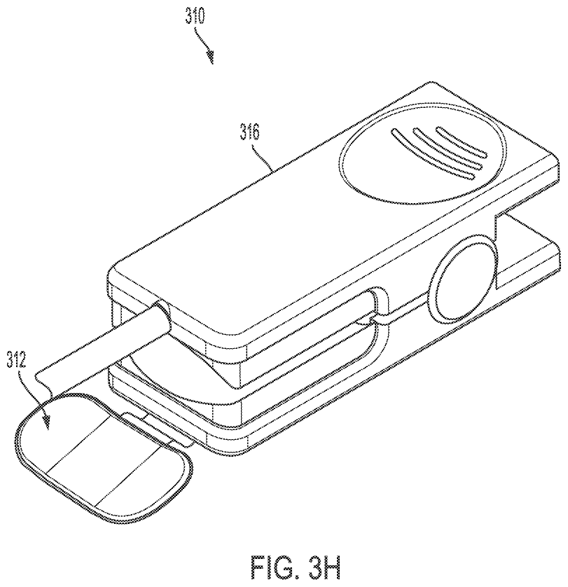

As previously described, the optical digit probes 112 of the present disclosure can include attachment devices for securing the optical digit probe 112 to the digit of the patient. Various examples of attachment devices that can be used with the optical digit probes 112 and attachment devices are shown in FIGS. 3A-12B. In some examples, the attachment device is permanently connected to or integrally formed with the optical digit probe 112, 201a. In other examples, the attachment device can be a separate part or device, which can be removably attached to the optical digit probe 112 by the patient or clinician prior to attaching the optical digit probe 112 to the digit to be monitored.

FIGS. 3A-3C show an example of an optical digit probe 310, which can be used with the medical systems and devices 110 of the present disclosure, including an attachment device 312, which is permanently connected to or an integral portion of the optical digit probe 310. FIGS. 3A and 3B show different views of the optical digit probe 310. FIGS. 3C and 3E-3G are drawings showing the optical digit probe 310 inserted onto fingers (e.g., a middle finger, little finger, index finger or ring finger) of a patient. FIG. 3H shows the attachment device 312 connected to a clip-type digit probe 310, similar to the probe shown in FIG. 2. The attachment systems, techniques, devices and/or fixation mechanisms described herein can be used in connection with any patient digit. For example, the attachment systems, techniques, devices and/or fixation mechanisms described herein can be used in connection with any finger, including index finger, ring finger, or little finger (fifth digit), among others.

The feature of permanently connecting, as described in examples herein, includes features whereby the attachment device 312 includes an attachment or fastening portion 314 that is fastened to an enclosure or digit housing 316 of the optical digit probe 310, making it difficult or impossible for a patient or clinician to inadvertently or intentionally detach the attachment device 312 from the digit housing 316 without destroying the overall structure for its intended purpose (e.g., monitoring physiological parameters of the patient via the patient's digit). For example, the attachment device 312 can be permanently attached to the digit housing 316 by adhesives or by mechanical fasteners, such as pins, screws, nails, staples, or thread (e.g., stitching).

Additionally or alternatively, the feature of permanently connecting the attachment device 312 to the housing 316 includes features whereby the attachment portion or device 312 is an integral part of the housing 316. For example, the attachment device 312 and the housing 316 can together be formed from a single mold and be based on a same material, e.g., a same flexible or rigid plastic material.

As shown in FIGS. 3A-3C and 3E-3H, the optical digit probe 310 comprises the digit housing 316. As previously described, the digit housing 316 can be formed from a rigid plastic (e.g., acrylonitrile butadiene styrene (ABS), polyester, polycarbonate, polypropylene, polyethylene, or polyethylene terephthalate). The digit housing 316 includes an external digit housing surface 318 and an internal digit housing surface 320. The digit housing 316 further includes a distal end 322, an open proximal end 324, and a cylindrical, annular, or tubular sidewall 326 extending between the open proximal end 324 and the distal end 322. The open proximal end 324 of the digit housing 316 is adapted for facilitating insertion of a digit of the patient into the digit housing 316. The digit can be, for example, a finger or toe of the patient. For example, the optical digit probe 310 can be configured for insertion onto a middle finger of the patient, as shown in FIG. 3C. The optical digit probe 310 can also be inserted onto a little finger (FIG. 3E), a ring finger (FIG. 3F), or a pointer or index finger (FIG. 3G). In other examples, the digit housing 316 can be configured to be worn on a big toe (hallux) of the patient or on another convenient extremity of the patient. For example, the systems, methods, and devices as described herein can be attached to any of the first toe, the second toe, the third toe, the fourth toe, or the fifth toe. In other examples, the optical digit probe 310 can be applied to other portions of the wearer's body, such as a wrist of the wearer, a lower arm of the wearer, an upper arm of the wearer, and/or a hand of the wearer.

Example dimensions of the digit housing 316 depend upon the type of digit being monitored by the optical digit probe 310. For an optical digit probe 310 configured to be worn on a patient's middle finger, the digit housing 316 can have an axial length of about 4 cm to about 8 cm. The open proximal end 324 of the digit housing 316 can have an outer diameter of about 3 cm to about 4 cm and an inner diameter of about 2 cm to about 3 cm. In some implementations, the dimensions of the digit housing 316 can be adjusted to accommodate varying digit sizes of the patient. For example, the inner diameter of the housing 316 can vary within a range of about 2 cm to about 3 cm when the patient inserts his or her middle finger into the housing 316. As noted, the dimensions depend on the patient's digit. In some implementations, the systems, methods, and devices as described herein can be configured to allow for fixation to any or all of the index finger (pointer finger or forefinger), middle finger, ring finger, little finger (pinky), or thumb. Accordingly, the dimensions may be different corresponding to the average person's dimensions for the foregoing patient digits.

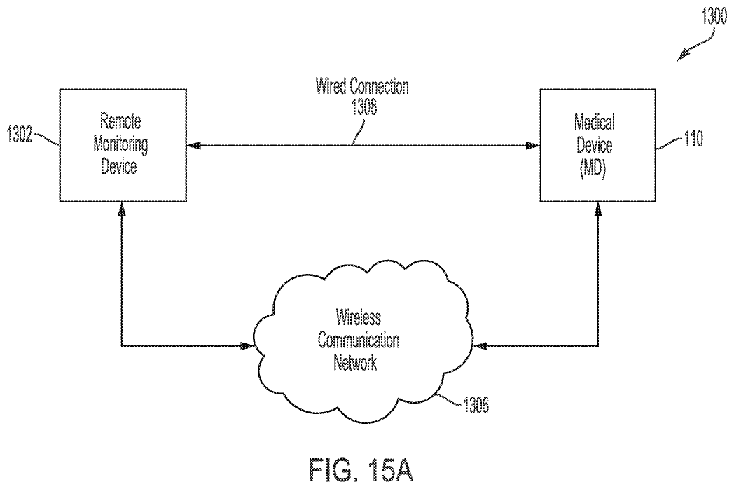

The optical digit probe 310 further comprises an optical sensor assembly 328 connected to and/or disposed in the digit housing 316 comprising an optical sensor configured to be positioned proximate to the digit of the patient to provide optical signals reflecting a physiological parameter of the patient. Various examples of physiological sensors for use in an optical digit probe 310 of the present disclosure are described herein in connection with FIGS. 13A-13C and 14. In some examples, the optical sensors of the assembly 328 of the present disclosure can be PPG or oximetry sensor configured to monitor certain predetermined blood metrics, e.g., one or more oxygen saturation measurements, for the patient. In other examples, the optical sensors of the assembly 328 can include sensors for measuring arterial vasomotor activity through the digit of the patient. Further, the optical signals provided by the optical sensors of the assembly 328 can reflect an arterial pulse waveform of the patient. As shown in FIGS. 3A-3C and 3E-3H, the optical sensor assembly 328 can be positioned on an upper or top portion of the digit housing 316, such that the optical sensor assembly 328 rests above the digit inserted in the digit housing 316. In some examples, the optical sensor assembly 328 can be a wired sensor assembly including a cable port 330 on a proximal end of the optical sensor assembly 328 sized to receive a connecting cable 134 (shown in FIGS. 13A-13C) for attaching the optical sensor assembly 328 to a wearable monitoring device, such as the wrist-worn monitor 118 shown in FIGS. 13A-13C.

In some examples, the optical digit probe 310 further comprises a pressure device, which is described in connection with FIG. 14 of the present disclosure. The pressure device can be disposed within an interior of the digit housing 316. As previously described, the pressure device can be configured to apply a substantially uniform sub-diastolic pressure field to the digit of the patient. The pressure device can be configured to facilitate unloading of arterial wall tension. Further, the pressure device can be configured to mitigate distal venous pooling or distention to avoid induction of venoarterial-mediated vasoconstriction.

The optical digit probe 310 further comprises the attachment device 312, which can be permanently connected to or integral with the digit housing 316, in the manner described above. As previously described, the attachment device 312 can be partially or entirely formed from a thermoplastic elastomer, such as silicone, polypropylene, low molecular weight polyethylene, synthetic rubber (e.g., polychloroprene) or natural rubber (e.g., isoprene). In other examples, portions of the attachment device 312 can be formed from more rigid plastic materials, such as acrylonitrile butadiene styrene (ABS), polyester, polycarbonate, polypropylene, high molecular weight polyethylene, or polyethylene terephthalate.

The attachment device 312 includes the housing fastening portion 314 connected to the external digit housing surface 318 of the digit housing 316 and a skin securement portion 332 extending from the housing fastening portion 314 configured to releasably couple the attachment device 312 to a skin surface of the wearer or patient. In some examples, the attachment or housing fastening portion 314 and the digit housing 316 are separate parts that are adhered together by an adhesive or fastener. For example, an adhesive, such as an acrylic adhesive or a low surface energy (LSE) adhesive, can be used to adhere the attachment or housing fastening portion 314 to the digit housing 316. Adhesives used for this application can include features such as high initial bond for immediate usability, good chemical and humidity resistance, −40° F. to 300° F. short-term temperature resistance, anti-lifting performance on curved surfaces, and no major surface preparation or primer application needed. As a specific example, the adhesive can include an acrylic adhesive such as 300LSE manufactured by 3M of Saint Paul, Minnesota, USA. For example, the attachment or housing fastening portion 314 can include an inwardly facing surfaces and an outwardly facing surface. The inwardly facing surface can be adhered to the external digit housing surface 318 of the digit housing 316. In particular, the inwardly facing surface can be permanently and immovably fixed to the external digit housing surface 318 of the digit housing 316.

In some examples, the housing fastening portion 314 can be provided on a distal end of the attachment device 312 and the skin securement portion 332 can be on a proximal end of the attachment device 312. In such cases, the attachment device 312 can further comprise a middle portion 340 between the housing fastening portion 314 and the skin securement portion 332. The middle portion 340 and/or housing fastening portion 314 can extend proximally over or beyond the open proximal end 324 of the digit housing 316, as shown in FIGS. 3A and 3B.

As shown in FIGS. 3A-3C and 3E-3H, the attachment device 312 can be an elongated or longitudinal member, such as a longitudinal attachment device, having a narrow distal end 334, a narrow proximal end 336, and longitudinal sides 338 extending therebetween. The attachment device 312 can be positioned or oriented relative to the digit housing 316 such that a longitudinal axis L1 of the elongated housing fastening portion 314 is substantially parallel to a longitudinal axis L2 of the digit housing 316. The longitudinal axis L1 of the attachment device 312 can correspond to an insertion direction of the digit of the patient into the digit housing 316.

The attachment device 312 further comprises the skin securement portion 332 including an inwardly facing surface 342 configured to contact the skin surface of the patient and an outwardly facing surface 344. In some examples, the inwardly facing surface 342 includes an adhesive, such a hydrogel and/or removable adhesive, for attaching the skin securement portion 332 to the skin surface of the patient. The skin securement portion 332 can be an elongated member having an axis L3. The axis L3 can be transverse or substantially transverse (e.g., about 90 degrees) relative to the axis L1, L2 of the digit housing 316 and housing fastening portion 314. In an example, the skin securement portion 332 can be configured to at least partially wrap about the digit of the patient, e.g. around a proximal phalange of the finger. In an example, the skin securement portion 332 can be configured to attach to a proximal phalange portion of the front of the hand (when viewed towards the palm).

FIG. 3D shows another example of an optical digit probe 310 including a digit housing 316 or housing portion comprising an external digit housing surface 318 and an internal digit housing surface 320. As in previous examples, the open proximal end 324 of the digit housing 316 is adapted for facilitating insertion of a digit of the patient into the digit housing 316. The optical digit probe 310 also includes the skin securement portion 332 configured to releasably couple the optical digit probe 310 to a skin surface of the patient. The optical digit probe 310 also includes an attachment or housing fastening portion 314 extending between the external digit housing surface 318 of the digit housing 316 or housing portion and the skin securement portion 332. As in previous examples, the optical digit probe 310 of FIG. 3D also includes the optical sensor assembly 328 disposed in the digit housing 316 or housing portion of the optical digit probe 310 comprising an optical sensor configured to be positioned proximate to the digit of the patient to provide optical signals reflecting the at least one physiological parameter of the patient.

As shown in FIG. 3D, the digit housing 316 and attachment or housing fastening portion 314 of the optical digit probe 310 can be integrally connected together forming a unitary or monolithic part. For example, the optical digit probe 310 can be a molded part formed, for example, by a single molding process, such as by a single injection molding process. In such cases, the digit housing 316, the skin securement portion 332, and the attachment or housing fastening portion 314 can be integrally formed, with the attachment or housing fastening portion 314 extending directly between the digit housing 316 and the skin securement portion 332. Following molding, an adhesive, such as a hydrogel and/or releasable adhesive, can be applied to the inwardly facing surface 342 of the skin securement portion 332 so that the skin securement portion 332 can be releasably coupled to the skin surface of the patient, as described in previous examples.

In order to use the optical digit probes 310 of FIGS. 3A-3H, the patient or clinician first obtains the optical digit probe 310 and removes any packaging from the optical digit probe 310. Once any packaging is removed, the patient inserts his or her digit, such as a middle finger, through the open proximal end 324 into the interior of the probe 310. In one example, the probe 310 is configured to receive a finger of the patient up to and including the distal and middle phalanges of the finger. The patient or clinician can then remove a protective cover or cover sheet from any adhesive covering the inwardly facing surface 342 of the skin securement portion 332. The patient or clinician can then wrap the skin securement portion 332 around a portion of the digit of the patient, e.g., a proximal phalange of a finger of the patient's hand, thereby securing the attachment device 312 and digit housing 316 to the digit of the patient shown in FIG. 3C.

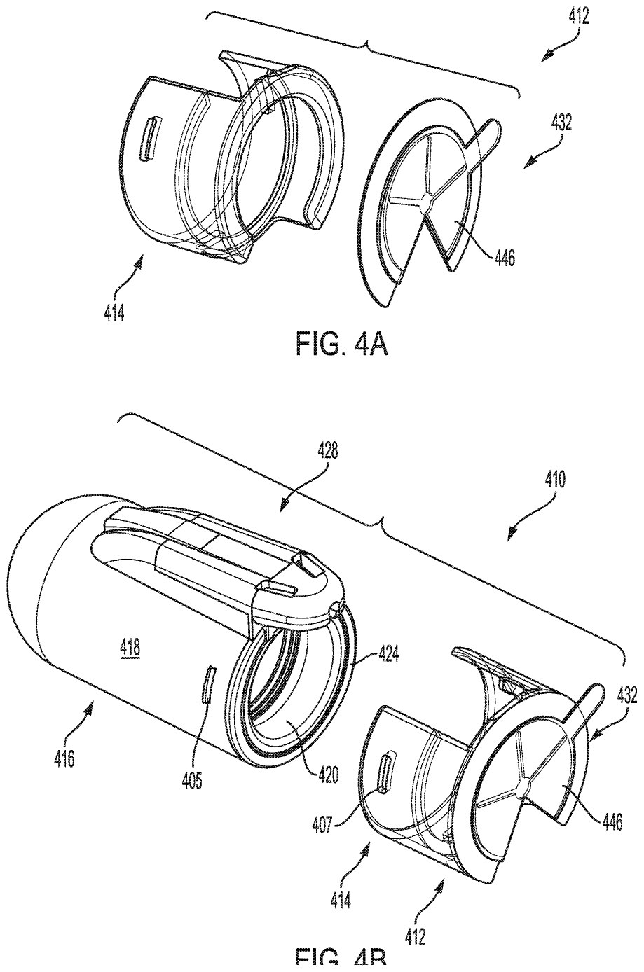

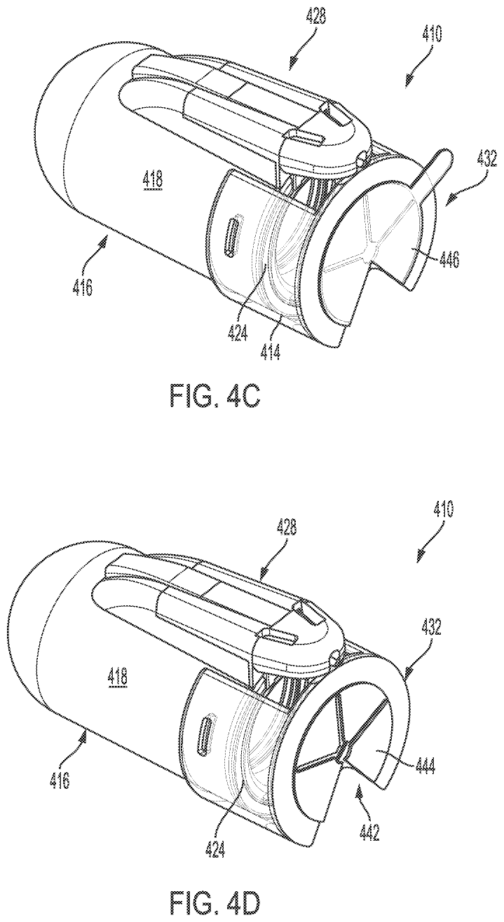

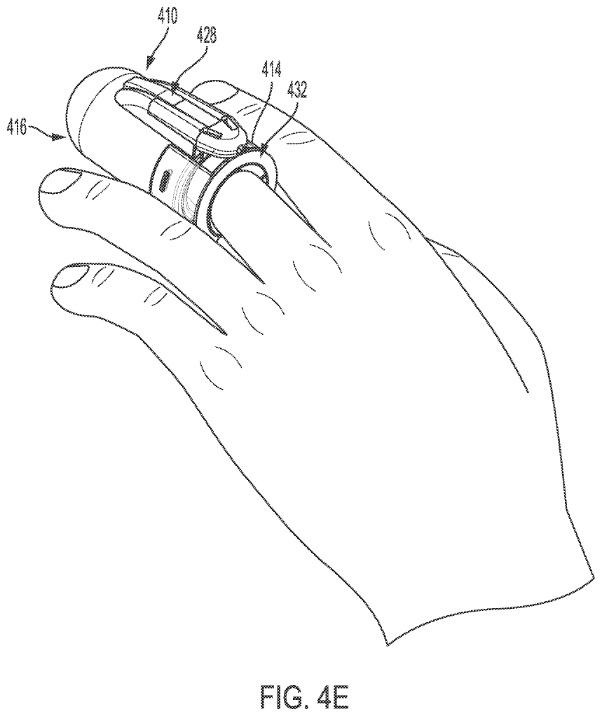

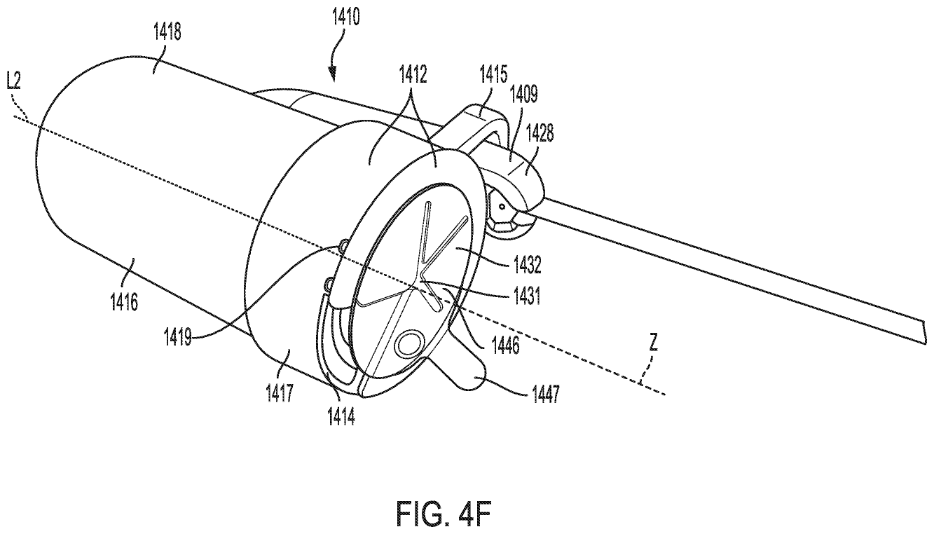

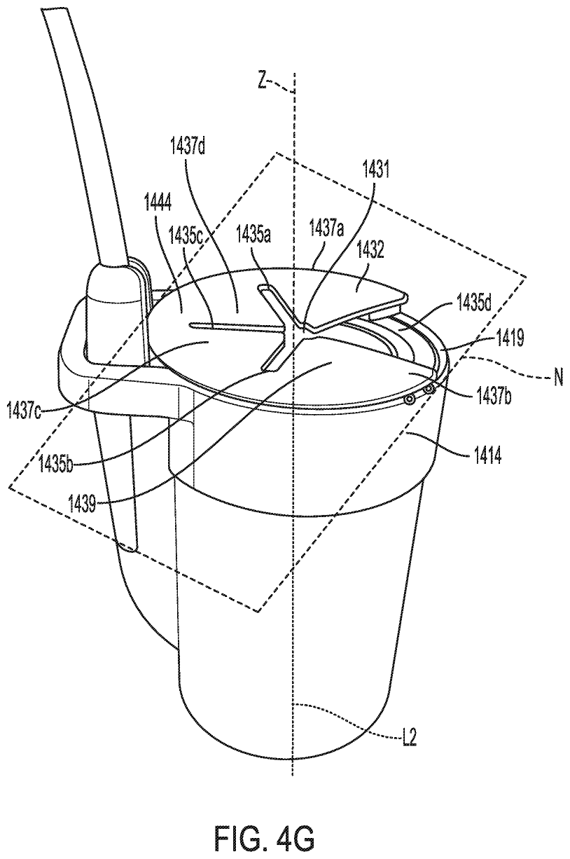

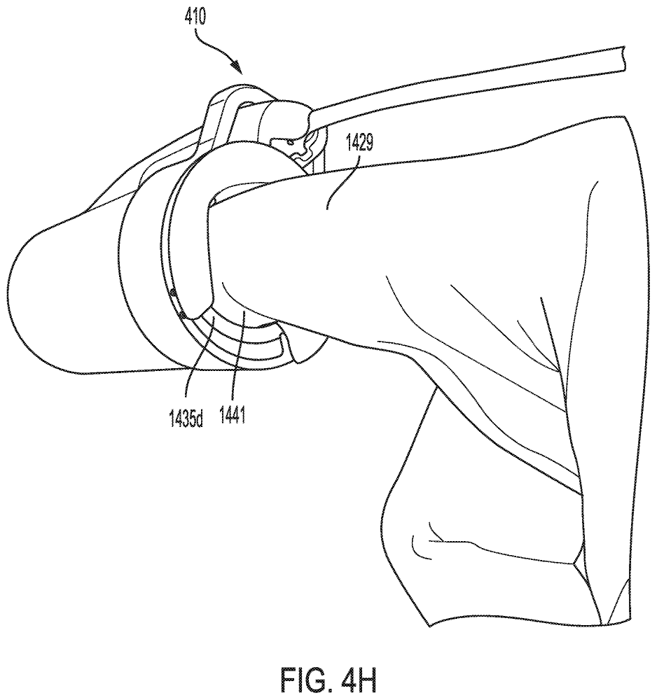

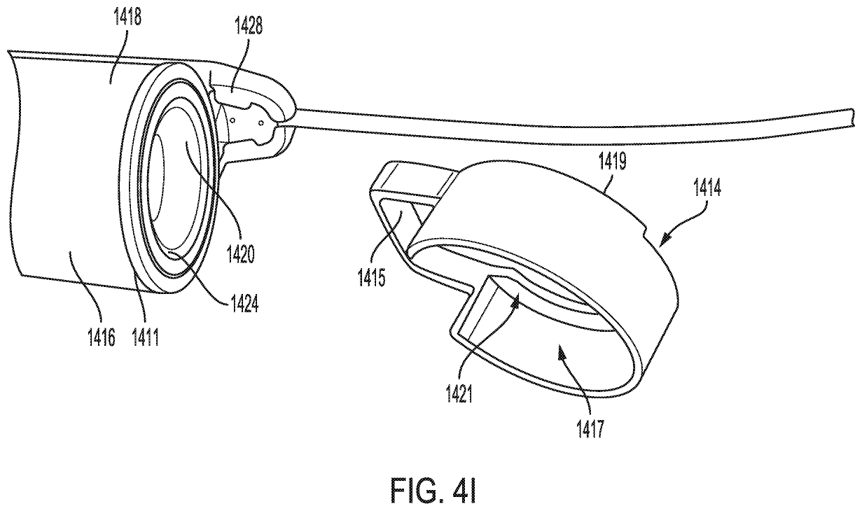

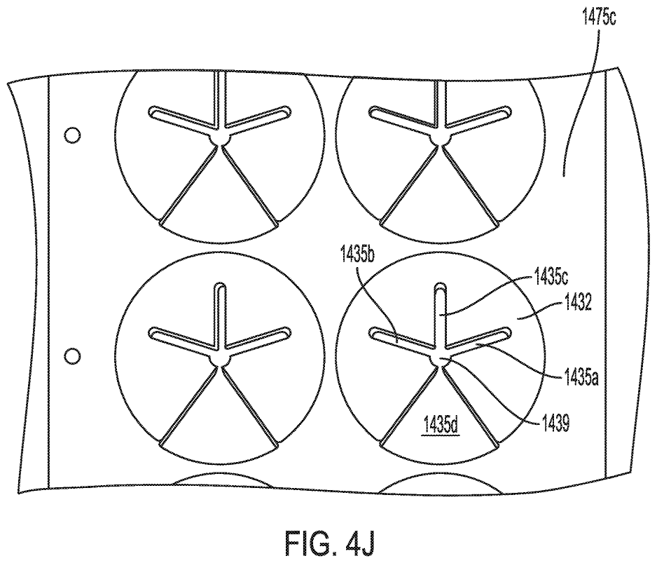

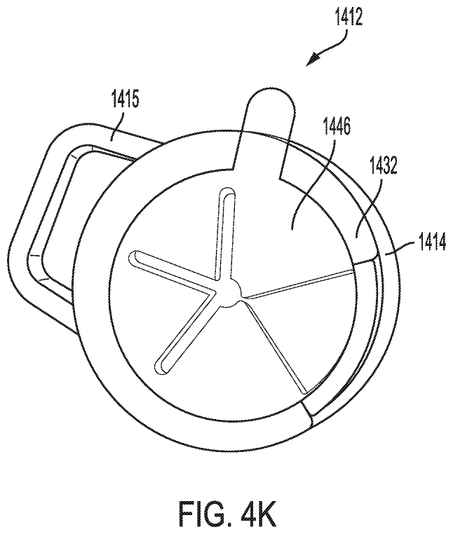

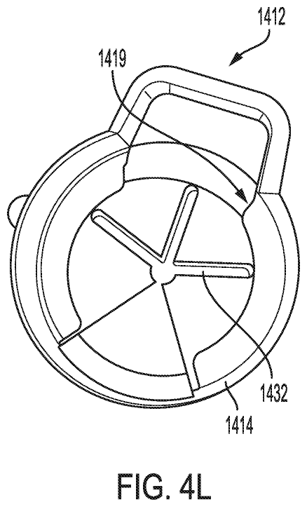

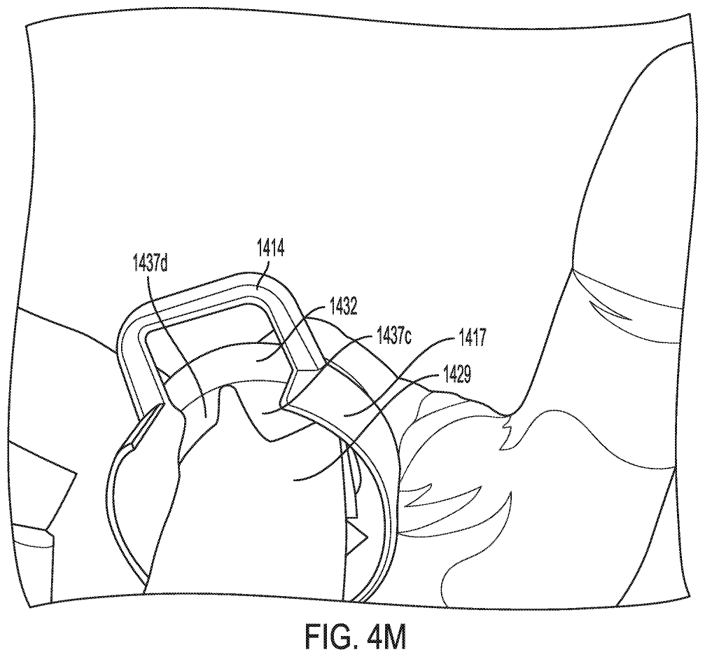

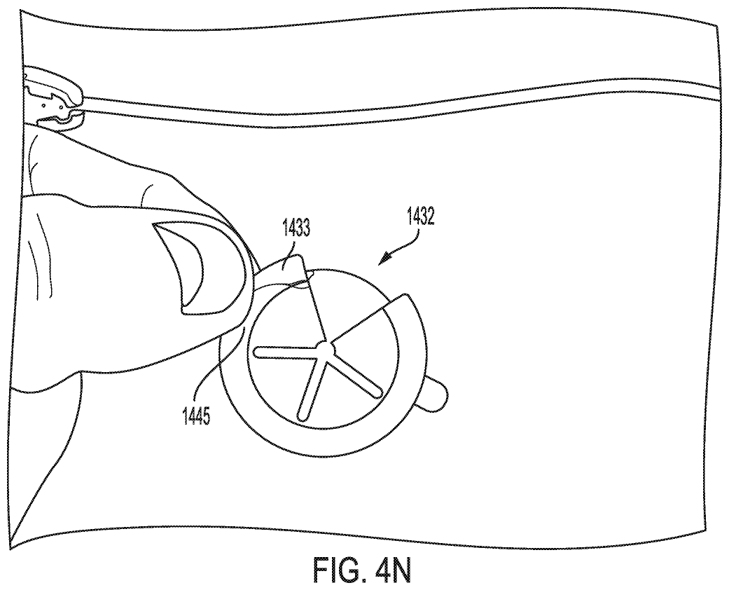

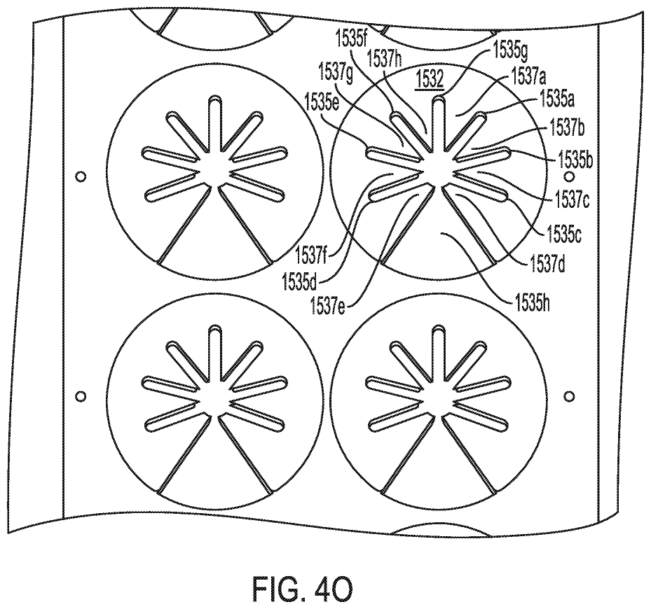

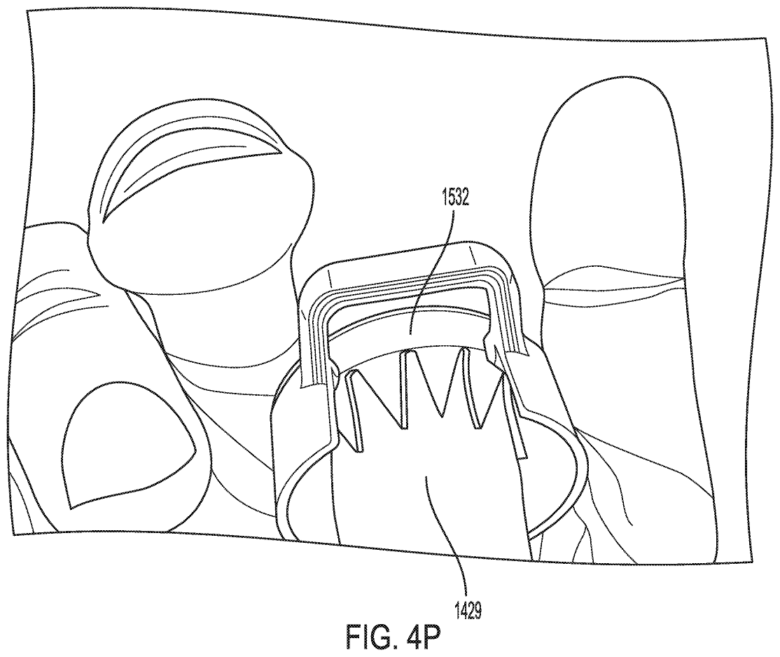

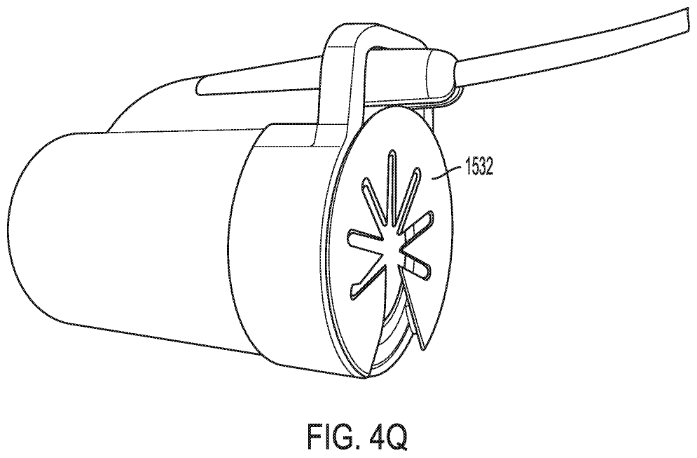

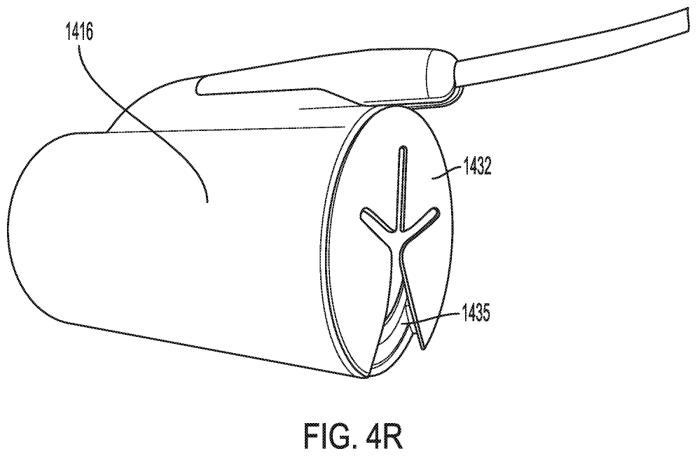

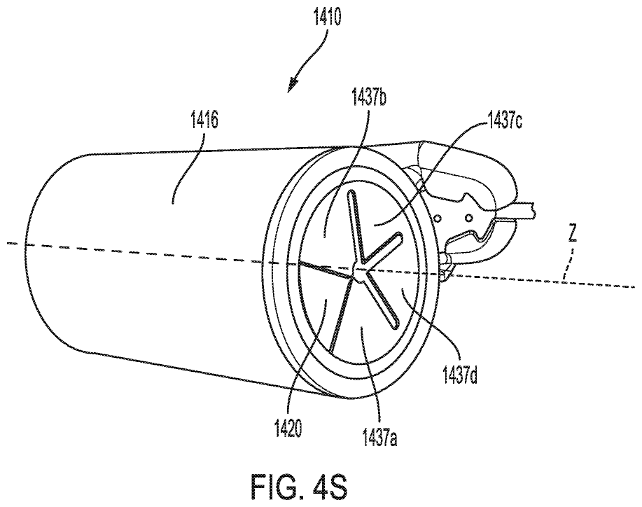

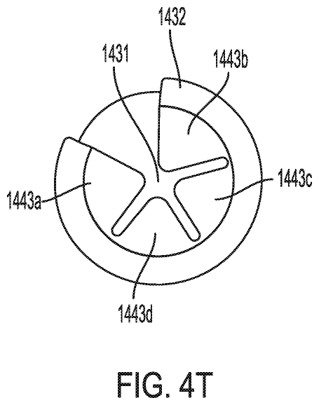

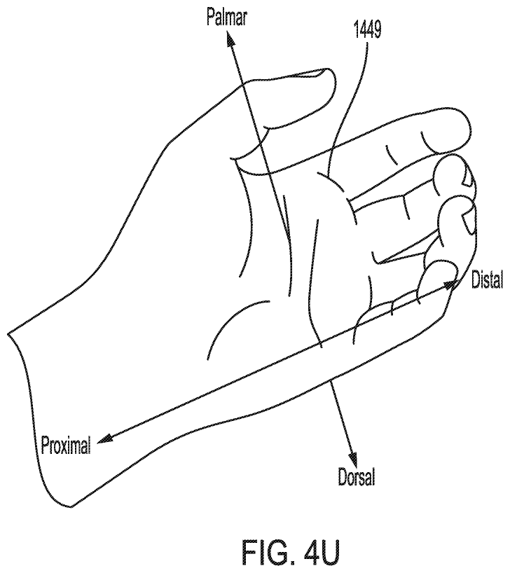

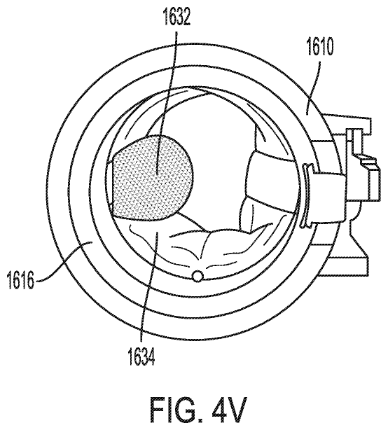

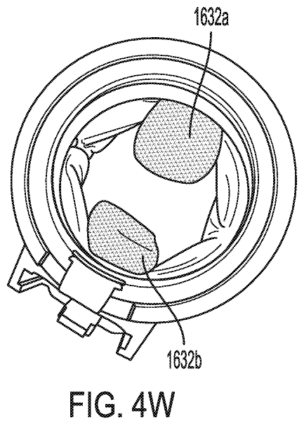

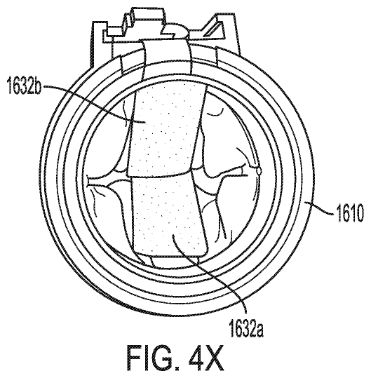

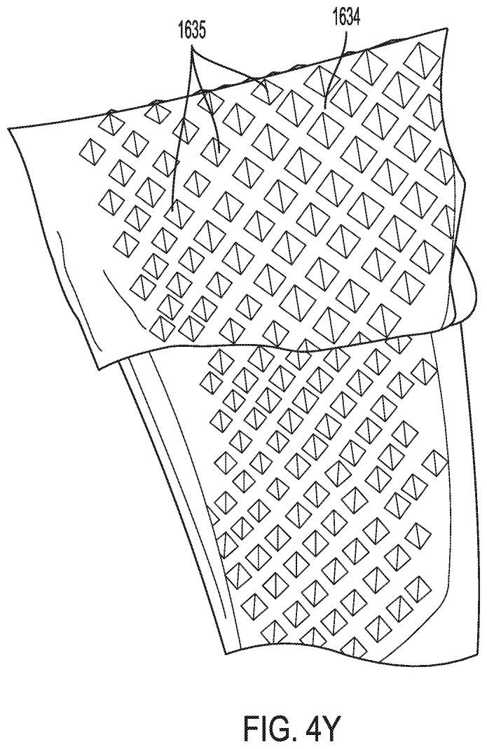

FIGS. 4A-4E show another example of an optical digit probe 410 and attachment device 412 including features of the present disclosure. Specifically, FIG. 4A is an exploded view of the attachment device 412 prior to attachment to the optical digit probe 410. FIG. 4B is an exploded view of the optical digit probe 410 and attachment device 412. FIGS. 4C and 4D are perspective views of the optical digit probe 410 and attachment device 412. FIG. 4E shows the optical digit probe 410 and attachment device 412 secured to a digit of a patient.

Unlike in previous examples, the attachment device 412 of FIGS. 4A-4E is configured to be removably connected over the open proximal end 424 of the digit housing 416. For example, the feature of removably connected includes features whereby the attachment device 412 is configured to be manually attached to and removed from the digit housing 416 when desired. For example, it is expected that the patient or clinician can attach the attachment device 412 to the digit housing 416 by pushing the attachment device 412 onto the digit housing 416 using one hand. In a similar manner, it is expected that the patient or clinician can remove the attachment device 412 from the digit housing 416 by grasping the attachment device 412 between fingers of one hand and pulling the attachment device 412 away from the digit housing 416. For example, the attachment device 412 can be attached to and removed from the digit housing 416 without using specially designed tools or other accessories, which may not always be available for the clinician or patient.

As shown in FIGS. 4A-4E, the optical digit probe 410 for noninvasive measurement of physiological parameters of the patient can include the digit housing 416 having the external digit housing surface 418 and the internal digit housing surface 420. The optical digit probe 410 can be configured for insertion onto a middle finger of the patient as shown in FIG. 4C. In other examples, the digit housing 416 can be configured to be worn on a big toe (hallux) of the patient or on another convenient extremity of the patient. In other examples, the optical digit probe 410 can be applied to other portions of the wearer's body, such as a wrist of the wearer, a lower arm of the wearer, an upper arm of the wearer, and/or a hand of the wearer.

Example dimensions of the digit housing 416 depend upon the type of digit being monitored by the probe 410. For an optical digit probe 410 configured to be worn on a patient's middle finger, the housing 416 can have an axial length of about 4 cm to about 8 cm. The open proximal end 424 of the digit housing 416 can have an outer diameter of about 3 cm to about 4 cm and an inner diameter of about 2 cm to about 3 cm. In some implementations, the dimensions of the digit housing 416 can be adjusted to accommodate varying digit sizes of the patient. For example, the inner diameter of the housing 416 can vary within a range of about 2 cm to about 3 cm when the patient inserts his or her middle finger into the housing 416. As noted, the dimensions depend on the patient's digit. In some implementations, the systems, methods, and devices as described herein can be configured to allow for fixation to any or all of the index finger (pointer finger or forefinger), middle finger, ring finger, little finger (pinky), or thumb. Accordingly, the dimensions may be different corresponding to the average person's dimensions for the foregoing patient digits.

The optical digit probe 410 of FIGS. 4A-4E also includes the removable attachment device 412. As in previous examples, the attachment device 412 includes a skin securement portion 432 extending from the digit housing 416 configured to releasably couple the attachment device 412 to the skin surface of the patient. The attachment device 412 can also include the housing fastening portion 414 configured to removably engage the external digit housing surface 418 of the digit housing 416 for securing the attachment device 412 to the digit housing 416. For example, the housing fastening portion 414 can be inwardly biased in order to form a snap fit connection with portions of the digit housing 416.

The optical digit probe 410 also includes the optical sensor assembly 428 connected to and/or disposed in the digit housing 416 comprising the optical sensor configured to be positioned proximate to the digit of the patient to provide optical signals reflecting a physiological parameter of the patient. For example, as shown in FIG. 4E, the optical sensor assembly 428 can be positioned on a top portion of the digit housing 416 so that the optical sensor assembly 428 is positioned above the digit of the patient when the digit is inserted into the digit housing 416.

As shown, for example, in FIG. 4D, when mounted to the digit housing 416, the attachment device 412 extends over the open proximal end 424 of the digit housing 416, such that the patient's digit contacts the skin securement portion 432 as it is inserted into the digit housing 416. More specifically, the housing fastening portion 414 can be an annular sleeve, ring, or collar, configured to be inserted over the open proximal end 424 of the digit housing 416. The skin securement portion 432 can comprise multiple flaps or triangular sheets extending radially inwardly from the housing fastening portion 414 over the open proximal end 424 of the digit housing 416. The flaps or triangular sheets can comprise an inwardly facing surface 442 (shown in FIG. 4D) and an outwardly facing surface 444 (shown in FIGS. 4D and 4E). The outwardly facing surface 444 is configured to press against or contact the patient's digit when the digit is inserted in the digit housing 416, as shown in FIG. 4E. The outwardly facing surface 444 can include or be covered with an adhesive for securing the skin securement portion 432 of the attachment device 412 to the patient's digit.

In some examples, as shown in FIGS. 4A-4C, the attachment device 412 also includes a removable cover 446, which is initially positioned over the outwardly facing surface 444 of the skin securement portion 432. The removable cover 446 protects the adhesive layer on the skin securement portion 432 preventing dirt, dust, or other debris from sticking to the adhesive. The removable cover 446 can also prevent dirt, dust, or other debris from entering the interior of the digit housing 416, which may affect signal fidelity of signals detected by the optical sensor assembly 428. The removable cover 446 can be removed by the patient or clinician shortly before inserting the digit housing 416 of the optical digit probe 410 onto the patient's digit.

Referring to FIGS. 4F-4I, an example implementation of an optical digit probe 1410 is shown in accordance with the present disclosure. Optical digit probe 1410 includes a digit housing 1416, an attachment device 1412 configured to couple thereto, and an optical sensor assembly 1428. Optical sensor assembly 1428 comprises similar and/or like features as described herein with respect to optical sensor assembly 428 of optical digit probe 410 (see, e.g., FIGS. 4A-4E and associated text). FIG. 4F shows optical digit probe 1410 in an assembled configuration. FIG. 4G shows optical digit probe 1410 with a front cover 1446, which is similar to cover 446, removed. FIG. 4H shows optical digit probe 1410 with a patient's finger 1429 inserted. FIGS. 4I-4N show detailed views of various components of attachment device 1412 and operations thereof. FIGS. 4O-4T show alternative examples of one of the components of attachment device 1412. FIG. 4U shows proximal, distal, palmer, and dorsal directions as described herein. Optical digit probe 1410 may be constructed with all of the same or similar structural features, functionalities, and uses discussed above with respect to optical digit probe 410, but can additionally or alternatively be constructed with some differences in design as further discussed below.

As best shown in FIGS. 4F-4L, digit housing 1416 of optical digit probe 1410 has an external surface 1418 and an internal surface 1420, and comprises an open proximal end 1424 (FIG. 4I) for receiving the patient's finger 1429 (FIG. 4H). Attachment device 1412 of optical digit probe 1410 includes a housing fastening portion 1414 which detachably couples to digit housing 1416, and a skin securement portion 1432 which detachably couples to housing fastening portion 1414. Housing fastening portion 1414 includes a U-shaped engagement portion 1415 configured to receive a first portion 1409 of digit housing 1416 of optical sensor assembly 1428, and a body portion 1417 defining a channel 1421 (FIG. 4I) configured to receive an external surface 1418 of a second portion 1411 of digit housing 1416 to secure (e.g., detachably couple, detachably mount, or detachably fix) attachment device 1412 to digit housing 1416. Body portion 1417 may be spring biased to snap onto external surface 1418, and/or or constructed to slide over external surface 1418 in an interference fit, a snap fit, and/or, for example, a pin and slot configuration such as pin 405 and slot 407 of optical digit probe 410 of FIG. 4B.

It will be appreciated that attaching body portion 1417 of housing fastening portion 1414 to digit housing 1416 and sliding optical sensor assembly 1428 through U-shaped engagement portion 1415 of housing fastening portion 1414 allows for increased positional alignment and orientation of optical sensor assembly 1428 relative to attachment device 1412. This increases alignment and orientation of optical sensor assembly 1428 relative to the patient's finger 1429 once skin securement portion 1432 is applied thereto (further discussed below). Attachment of U-shaped engagement portion 1415 to optical sensor assembly 1428 additionally rotatably fixes housing fastening portion 1414 to optical sensor assembly 1428.

Skin securement portion 1432 detachably couples (e.g., detachably mounts or detachably fixes) to a front surface 1419 (FIG. 4I) of housing fastening portion 1414 via an adhesive layer operatively disposed on a rear surface 1433 of skin securement portion 1432 (FIG. 4N) to assemble attachment device 1412. In particular, rear lining 1445 (FIG. 4N) is peeled back to expose rear surface 1433 of skin securement portion 1432, which has a layer of adhesive, and rear surface 1433 is stuck to front surface 1419 (FIG. 4I) of housing fastening portion 1414. Attachment device 1412 is then in an assembled configuration as shown in the front and rear views of FIGS. 4K and 4L, respectively.

In this assembled configuration, attachment device 1412 comprises a longitudinal axis Z extending through housing fastening portion 1414 and skin securement portion 1432. Skin securement portion 1432 comprises a plurality of flexible members (further discussed below) which are arranged as bendable flaps about a center 1431. The flaps lie may lie in a plane N which is transverse or perpendicular to the longitudinal axis Z of attachment device 1412. When skin securement portion 1432 is detachably coupled to housing 1416, transverse plane N in which the bendable flaps lie is also intersected by a longitudinal axis L2 of optical digit probe 1410 (FIG. 4G), which may be collinear with longitudinal axis Z of attachment device 1412 in this assembled configuration.

Attachment device 1412 may be equipped with a removable front cover 1446 stuck onto the front of skin securement portion 1432, and removable with a tab 1447. Front cover 1446 is initially positioned over an outwardly facing surface 1444 (FIG. 4G) of skin securement portion 1432. Removable front cover 1446 may be made from a plastic material, and, similar to removable cover 446, configured to prevent entry of dirt, dust, or other debris from entering skin securement portion 1432 and/or the interior of housing 1416 prior to use. Removable cover 1446 can be removed by the patient or clinician shortly before use.

It will be appreciated that skin securement portion 1432 can be detachably coupled (e.g., detachably mounted or detachably fixed) to housing fastening portion 1414 before or after housing fastening portion 1414 is detachably coupled to digit housing 1416. Additionally, attachment device 1412 may come preassembled for the patient, on or off of digit housing 1416.

Skin securement portion 1432 is configured to releasably couple (e.g., movably couple) housing fastening portion 1414 of attachment device 1412 (and by extension, digit housing 1416) to the skin surface of the patient's finger 1429. In certain examples, skin securement portion 1432 is disposable and replaceable each night by the patient with a new skin securement portion 1432 (FIG. 4J). The patient can simply pull skin securement portion 1432 off of front surface 1419 of housing fastening portion 1414, remove removable rear lining 1445 from rear surface 1433 of a new skin securement portion 1432, and attach the new skin securement portion 1432 to front surface 1419 (FIG. 4I) of housing fastening portion 1414. In this manner, optical digit probe 1410 can be used for multiple nights (e.g., two, three, four nights, etc.) by simply replacing skin securement portion 1432. As shown in FIG. 4G, skin securement portion 1432 defines slots 1435a, 1435b, 1435c and opening 1435d which border flexible members (e.g., tapered regions 1437a, 1437b, 1437c, 1437d) configured to bend and contact/grip the patient's finger 1429 when finger 1429 is inserted through skin securement portion 1432 and housing fastening portion 1414 (FIG. 4M). While tapered regions 1437a, 1437b, 1437c, 1437d are triangular in shape, other shapes may be utilized. As an example, certain tapered regions 1443a-d may instead have a convex or arc shape, and taper toward center 1431 as shown in FIG. 4T.

As shown in FIG. 4M, flexible tapered regions 1437a, 1437b, 1437c, 1437d bend to extend radially to contact finger 1429, and distally in the axial (e.g., longitudinal) direction of finger 1429 along a particular length thereof, substantially parallel thereto. Each tapered region 1437a, 1437b, 1437c, 1437d is bordered by a respective pair of adjacent slots of the plurality of slots 1435a, 1435b, 1435c, or by one of slots 1435a, 1435b, 1435c and opening 1435d. As shown in FIGS. 4F and 4G, prior to the patient inserting finger 1429, flexible tapered regions 1437a, 1437b, 1437c, 1437d are in a relaxed state, and extend transverse to (e.g., perpendicular to) a longitudinal axis ‘Z’ (which extends longitudinally through attachment device 1412, and in the assembled configuration of FIG. 4F, digit housing 1416 of optical digit probe 1410), and thus is configured to minimize cross sectional space in the axial direction. For example, in implementation, the attachment device 1412 is configured to increase an axial length of optical digit probe 1410 by approximately the overall thickness of skin securement portion 1432. In certain example implementations, the proximal end of housing fastening portion 1414 may lie flush with or distal of the proximal edge of proximal opening 1424 of housing 1416 to limit any increase in the axial length of optical digit probe 1410 to the thickness of skin securement portion 1432.

Once the patient's finger 1429 is inserted, tapered regions 1437a, 1437b, 1437c, 1437d bend in the longitudinal direction of the finger. A substantial portion of each tapered region, when bent by finger 1429, contacts finger 1429. Skin securement portion 1432 thus functions to provide significant surface area in contact with the patient's inserted finger 1429 while adding very little axial length to optical digit probe 1410.

It will be appreciated that implementation herein is configured to offer a predetermined contact area for frictional resistance to movement of the patient's finger in the proximal direction. Tapered regions 1437a, 1437b, 1437c, 1437d bend in the distal direction, and thus allow for relative movement of the patient's finger 1429 in the distal direction, but resist movement of finger 1429 in the proximal direction. In particular, proximal movement of finger 1429 forces tapered regions 1437a, 1437b, 1437c, 1437d to bunch up around the finger 1429, thereby creating additional frictional resistance to such proximal movement. In this manner, bendable tapered regions 1437a, 1437b, 1437c, 1437d provide a ratcheting function, with lower resistance to movement of finger 1429 in the distal direction when compared to greater resistance to movement of finger 1429 in the proximal direction (e.g., to proximal translation of finger 1429 out of open proximal end 1424).

It will be appreciated that tapered regions 1437a, 1437b, 1437c, 1437d similarly resist rotational movement of finger 1429 when engaged with finger 1429 as shown in FIG. 4H. In certain example implementations, tapered regions 1437a, 1437b, 1437c, 1437d may be spring biased toward the radially directed orientation shown in FIGS. 4F and 4G. However, even without such spring bias, the radially outer base portion of each tapered region 1437a, 1437b, 1437c, 1437d is compressed upon insertion of finger 1429, thereby creating an arcuate pressurized contact area at the base of each tapered region which resists rotation of the finger 1429. The triangular shape and tapering of tapered regions 1437a, 1437b, 1437c, 1437d from their respective radially outer base portions to their respective radially inner tip portions provides ample material at the base (e.g., at radially outer base portions) of tapered regions 1437a, 1437b, 1437c, 1437d to absorb pressure when the patient's finger 1429 is inserted. The tapering of tapered regions 1437a, 1437b, 1437c, 1437d extending toward and along the patient's finger provides significant contact area as described above, while also avoiding a bunching up of material as the patient's finger 1429 is inserted into optical digit probe 1410.

In other example implementations, tapered regions 1437a, 1437b, 1437c, 1437d may be elastically deformable. In yet other examples, tapered regions 1437a, 1437b, 1437c, 1437d may be provided with gel (e.g., hydrogel) on a front proximal side thereof to contact finger 1429. It will be appreciated that other shapes and sizes of such regions and slots may be utilized. Skin securement portion 1432 also defines a centralized hole 1439 (FIG. 4J) at the radially inner end (base) of slots 1435a, 1435b, 1435c and opening 1435d, and in communication therewith. Hole 1439 may be circular in shape to round out the radially inner tips of tapered regions 1437a, 1437b, 1437c, 1437d and prevent them from being too sharp against the user's skin. Hole 1439 also provides space for tapered regions 1437a, 1437b, 1437c, 1437d to fold inward in the distal direction as finger 1429 is inserted distally, and may be sized to reduce the total material of tapered regions 1437a, 1437b, 1437c, 1437d bent by finger 1429. Central hole 1439 may be sized to increase or decrease the total volume of material to accommodate variance in the width of digits (e.g., fingers or toes) of patients, and thus, the pressure exerted by tapered regions 1437a, 1437b, 1437c, 1437d on the patient's digit 1429.

Central hole 1439 may thus be sized to prevent patient discomfort while still allowing skin securement portion 1432 to resist proximal and rotational movement of the patient's finger 1429 relative to optical digit probe 1410. In certain embodiments, a plurality of skin securement portions 1432 may be provided with different sized central holes 1439 to accommodate different digit sizes. The patient's finger may be measured in advance, or the patient may be given authorization to switch the skin securement portion 1432 to select a tighter or looser fitting one. Hole/cutout 1435d defined in skin securement portion 1432 may be formed as an enlarged wedge-shaped gap on a lower region of skin securement portion 1432. Cutout 1435d is configured to comfortably receive fatty tissue in a bottom portion (e.g., a palmer/volar region of a proximal phalange 1449 as shown in FIG. 4U) of the patient's finger 1429, approximately below and proximal of the patient's middle knuckle. Volar region or palmer region 1449 may be received in hole 1435d.

Without enlarged wedge-shaped cutout/slot 1435d, fatty tissue 1441 (FIG. 4H) in the volar region of the proximal phalange of the patient's finger 1429 may be compressed and pushed rearward (i.e., proximally), depending on the size of finger 1429 inserted, as the finger 1429 is inserted distally. Tapered regions 1437a, 1437b, 1437c, 1437d are configured to be applied to, for example, a proximal phalange of finger 1429. Enlarged wedge-shaped cutout 1435d helps avoid creating additional pressure on blood vessels in finger 1429, which could otherwise effect the peripheral arterial tone signal derived from measurements at the distal region of finger 1429 by optical sensor assembly 1428. In fact, when finger 1429 is inserted, minimal to no pressure is exerted on fatty tissue 1441, which reduces the overall pressure to finger 1429 as a whole. Enlarged cutout 1435d may additionally be formed at other regions where slots 1435a, 1435b, 1435c are shown.

Other shapes and sizes of slots, holes, and flexible regions may be utilized. By way of example, as shown in FIGS. 4O-4Q, an alternative skin securement portion 1532 includes eight tapered flexible regions 1537a-1537h instead of four. Skin securement portion 1532 defines seven slots 1535a-g and enlarged cutout/hole 1535h. As shown in FIG. 4P, tapered regions 1537a-1537h bordered by slots 1535a-g and hole 1535h similarly extend longitudinally in the distal direction with finger 1429.

In certain example implementations, housing fastening portion 1414 may be eliminated, and skin securement portion 1432 may be attached directly to digit housing 1416 of optical digit probe 1410. In other words, skin securement portion 1432 may be attached directly to open proximal end 1424 of digit housing 1416 as shown in FIG. 4R without using housing fastening portion 1414, and the patient's finger may be inserted directly through skin securement portion 1432 and into digit housing 1416.

In yet other example implementations, housing fastening portion 1414 and/or skin securement portion 1432 may be integrally formed with digit housing 1416 as one piece as shown in FIG. 4S, and flexible regions 1437a, 1437b, 1437c, 1437d may be configured to radially extend from internal surface 1420 of digit housing 1416, perpendicular to axis Z, which extends through optical digit probe 1410 in the assembled configuration. In such embodiments, no detachable pieces are necessary.

Referring to FIG. 4V, in another example implementation, an optical digit probe 1610 includes a skin securement portion formed as a grip 1632 operatively disposed inside optical digit probe 1610. Grip 1632 is attached to one side of the external surface of a membrane 1634 (e.g., a radially inner membrane) whose radially inner gripping surface faces the inside of a housing 1616 of optical digit probe 1610. Grip 1632 extends longitudinally within housing 1616 along membrane 1634, and is configured to releasably couple (e.g., movably couple) a patient's finger 1429 when inserted into optical digit probe 1610 as similarly described above with respect to optical digit probe 1410. Grip 1632 may additionally or alternatively extend circumferentially around the inside of housing 1616 along the external surface of membrane 1634, and may additionally or alternatively be operatively disposed inside membrane 1634 with its radially inner gripping surface still configured to releasably couple to a patient's finger 1429 through membrane 1634.

Grip 1632 and membrane 1634 may both be made of a synthetic rubber such as nitrile. Membrane 1634 need not have elastic properties as it merely transfers/applies pressure to finger 1429 when finger 1429 is inserted into probe 1610. Grip 1632 is configured with a gripping surface which, when pressed against finger 1429, resists movement of finger 1429 relative to probe 1610, and in particular, proximal translation of finger 1429 relative to probe 1610. Such gripping surface may use, for example, hooks, protrusions, burrs, and/or loops with cutouts configured to grip the surface of finger 1429. The gripping surface may flexible or rigid, and made from a synthetic rubber such as nitrile, and additionally or alternatively from nylon, polyester, and/or other fabric. In certain example implementations, grip 1632 may be configured with very fine hooks for better gripping, and/or with each hook angled or defining a proximal cutout to resist proximal movement of finger 1429 out of probe 1610 more than distal movement of finger 1429 into probe 1410. It will be appreciated that resistance to longitudinal translation (e.g., proximal translation) of finger 1429 relative to probe 1410 is desired during testing overnight, particularly when a uniform pressure field is applied to finger 1429 as described with respect to various example implementations herein. Grip 1632 similarly resists rotational movement of finger 1429 relative to probe 1610, which also improves data collection and reliability during probe operation.

Optical digit probe 1610 may be configured to apply a uniform pressure field to portions (e.g., a distal two thirds) of finger 1429 during testing using one or more pockets, fluids, and elastic members or membranes formed from a flexible protective material as described with respect to other example implementations herein. One such example is illustrated in FIG. 14, further described below. In certain implementations, the uniform pressure field applied to finger 1429 may also compress grip 1632 onto finger 1429 to help maintain longitudinal positioning of finger 1429 relative to optical digit probe 1610, and thereby resist proximal movement of finger 1429 out of housing 1616.

Referring to FIG. 4W, in one implementation, skin securement portion 1632 may include two or more oppositely facing grips 1632a, 1632b inside optical digit probe 1610 configured to releasably couple to opposite sides of the patient's finger 1429. Grips 1632a, 1632b may both be formed, for example, with hooks, protrusions, burrs, and/or loops with cutouts. In certain implementations, grips 1632a, 1632b may be both made with hooks. Referring to FIG. 4X, in other implementations, oppositely facing grips 1632a, 1632b of skin securement portion 1632 may be made from an adhesive acrylic foam. Referring to FIG. 4Y, in yet other example implementations, membrane 1634 to which grip(s) 1632a, 1632b are attached may additionally or alternatively be formed with a synthetic rubber such as nitrile, and configured with numerous tapered members or protrusions 1635 of similar or varying shapes for further limiting movement (e.g., proximal longitudinal translation and/or rotation) of finger 1429 relative to optical digit probe 1610. In certain implementations, skin securement portion 1632 may comprise membrane 1634 with protrusions 1635 and/or grip 1632, or combinations thereof.

It will be appreciated that in certain implementations, skin securement portion 432 (FIG. 4D) and/or skin securement portion 1432 (FIG. 4F) may be used together with grip 1632, membrane 1634 with protrusions 1635, and/or other attachment mechanisms described herein to releasably couple a patient's finger to the various optical digit probes described herein.

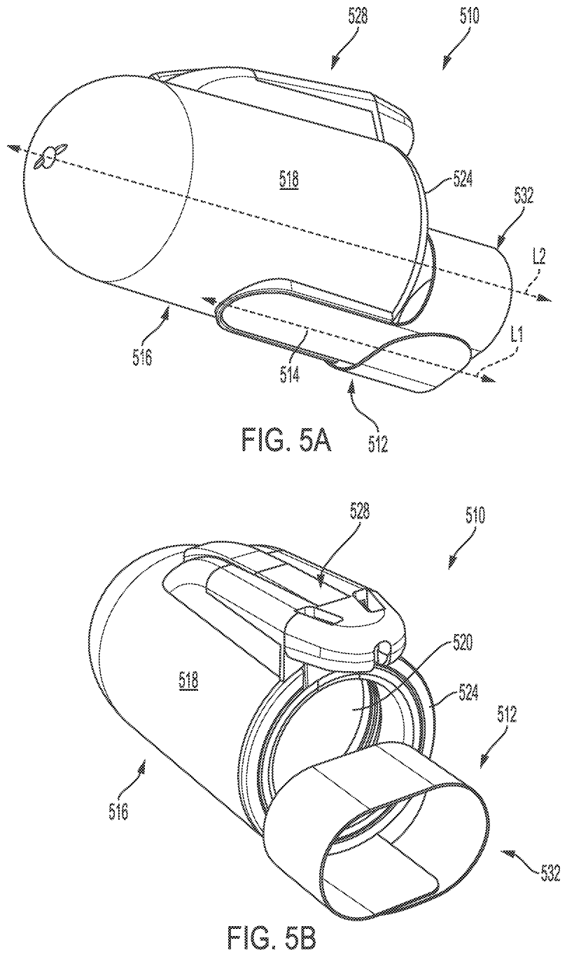

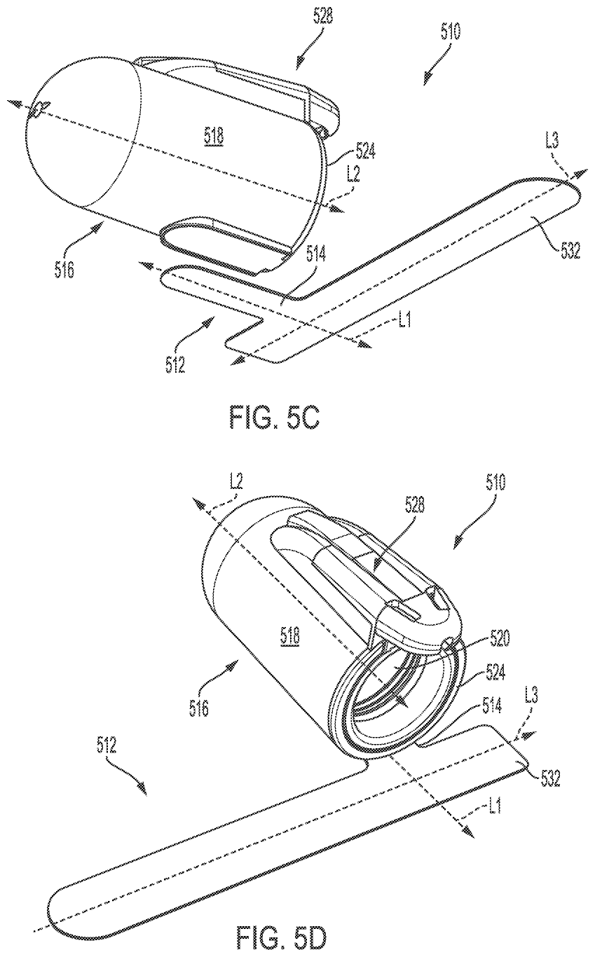

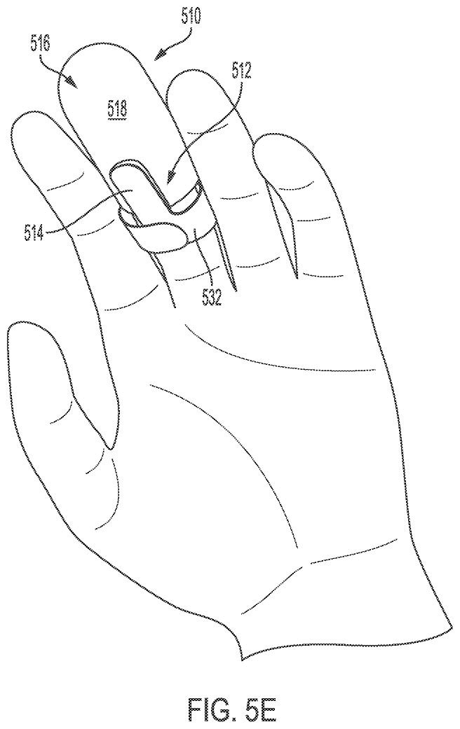

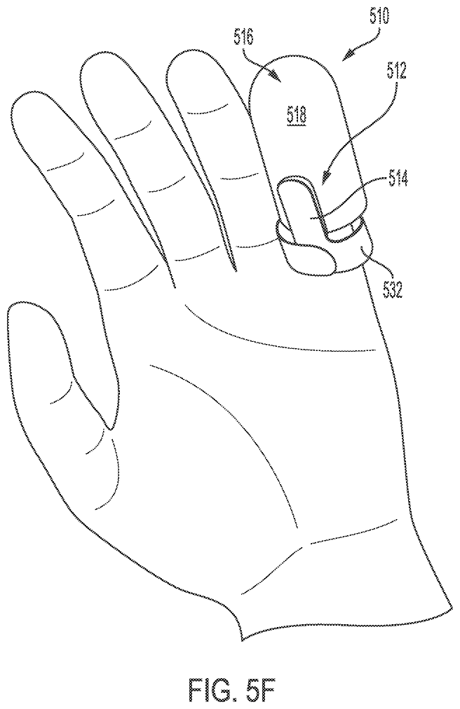

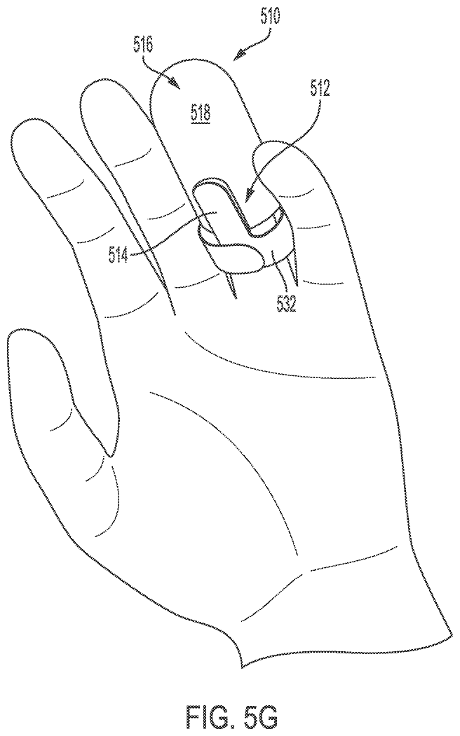

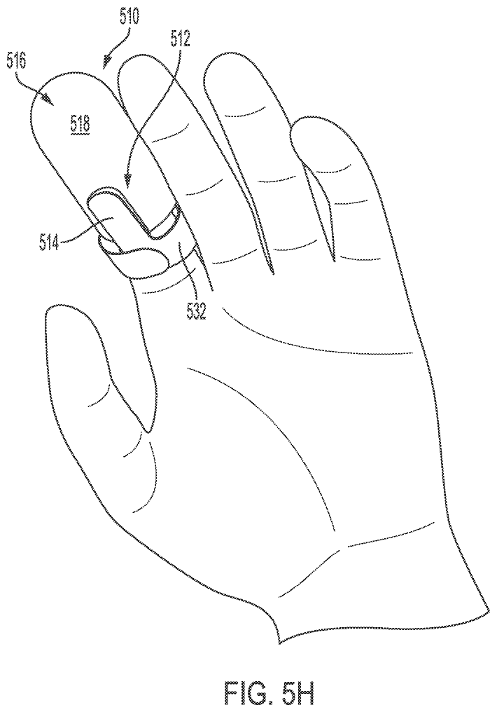

FIGS. 5A-9C show additional examples of optical digit probes 510, 610, 710, 810, 910 including a digit housing 516, 616, 716, 816, 916, and an attachment device 512, 612, 712, 812, 912 connected to an external surface 518, 618, 718, 818, 918 of the digit housing 516, 616, 716, 816, 916. In these examples, the attachment device 512, 612, 712, 812, 912 extends proximally from the digit housing 516, 616, 716, 816, 916 and is secured to a proximal portion of the digit. As previously described, the digit can be a finger of the patient, such as a middle finger of the patient as shown in FIGS. 5E, 6C, and 9C.

The optical digit probes 510, 610, 710, 810, 910 can be configured for insertion onto a middle finger, ring finger, index or pointer finger, or little finger of the patient. In other examples, the digit housings 516, 616, 716, 816, 916 can be configured to be worn on a big toe (hallux) of the patient or on another convenient extremity of the patient. In other examples, the optical digit probe 510, 610, 710, 810, 910 can be applied to other portions of the wearer's body, such as a wrist of the wearer, a lower arm of the wearer, an upper arm of the wearer, and/or a hand of the wearer.

Example dimensions of the digit housing 516, 616, 716, 816, 916 depend upon the type of digit being monitored by the probe 510, 610, 710, 810, 910. For an optical digit probe 510, 610, 710, 810, 910 configured to be worn on a patient's middle finger, the housing 516, 616, 716, 816, 916 can have an axial length of about 4 cm to about 8 cm. The digit housing 516, 616, 716, 816, 916 can have an outer diameter of about 3 cm to about 4 cm and an inner diameter of about 2 cm to about 3 cm. In some implementations, the dimensions of the digit housing 516, 616, 716, 816, 916 can be adjusted to accommodate varying digit sizes of the patient. For example, the inner diameter of the housing 516, 616, 716, 816, 916 can vary within a range of about 2 cm to about 3 cm when the patient inserts his or her middle finger into the housing 516, 616, 716, 816, 916. As noted, the dimensions depend on the patient's digit. In some implementations, the systems, methods, and devices as described herein can be configured to allow for fixation to any or all of the index finger (pointer finger or forefinger), middle finger, ring finger, little finger (pinky), or thumb. Accordingly, the dimensions may be different corresponding to the average person's dimensions for the foregoing patient digits.

More specifically, as previously described, the digit housing 516, 616, 716, 816, 916 of the optical digit probe 510, 610, 710, 810, 910 is configured to receive about a distal-most two thirds of the patient's digit. A proximal-most third of the digit is not received within the digit housing 516, 616, 716, 816, 916. For example, the digit housing 516, 616, 716, 816, 916 can be sized to receive a first or distal portion and a second or middle portion of the patient's digit. A third or proximal portion of the digit can be outside of the digit housing 516, 616, 716, 816, 916. Portions of the patient's digit can be referred to as a phalanges of the patient's hand or foot. For example, the first or distal portion of the digit can refer to the portion of the digit that surrounds, encloses, or covers the distal phalanges bone of the patient's hand or foot. The second or middle portion of the digit can refer to the portion of the digit that surrounds, encloses, or covers the intermediate or middle phalanges of the patient's hand or foot. The third or proximal portion of the digit can refer to the portion of the digit that encloses, surrounds, or covers the proximal phalanges bone of the digit, which is connected to a metacarpal bone of the patient's hand or a metatarsal bone of the patient's foot.

As shown in FIGS. 5A-5H, in some examples, the optical digit probe 510 for noninvasive measurement of physiological parameters of a patient comprises the digit housing 516 and the attachment device 512. Specifically, FIGS. 5A and 5B are perspective views of the optical digit probe 510 and attachment device 512 showing the attachment device 512 in an in-use position. FIGS. 5C and 5D are perspective views of the optical digit probe 510 and attachment device 512 showing the attachment device 512 in a pre-use position. FIG. 5E shows the optical digit probe 510 and attachment device 512 attached to a middle finger of the patient. FIG. 5F shows the optical digit probe 510 and attachment device 512 attached to a little finger of the patient. FIG. 5G shows the optical digit probe 510 and attachment device 512 attached to a ring finger of the patient. FIG. 5H shows the optical digit probe 510 and attachment device 512 attached to a pointer or index finger of the patient.

As in previous examples, the digit housing 516 includes an external digit housing surface 518 and an internal digit housing surface 520. A proximal end 524 of the digit housing 516 is adapted for facilitating insertion of a first portion and a second portion of a digit of the patient into the digit housing 516. For example, as previously described, the digit housing 516 can be sized so that the distal-most two thirds, such as the distal phalanges and the middle phalanges, can be inserted into the interior of the digit housing 516.

The attachment device 512 includes the housing fastening portion 514 connected to the external digit housing surface 518 of the digit housing 516 and the skin securement portion 532 configured to releasably couple the attachment device 512 to the skin surface of a third or proximal portion of the digit of the patient, such as to the third portion or proximal phalanges of the digit. For example, the skin securement portion 532 can be configured to partially or completely circumferentially enclose the third portion or proximal phalanges of the digit of the patient for securing the optical digit probe 510 to the digit of the patient.

As in previous examples, the optical digit probe 510 also includes the optical sensor assembly 528 disposed in and/or on the digit housing 516 configured to be positioned proximate to the digit of the patient to provide optical signals reflecting the physiological parameter of the patient. For example, as previously described, the optical sensor assembly 528 can be positioned on a top portion of the digit housing 516, such that the optical sensor assembly 528 is above the digit inserted into the digit housing 516.

As shown in FIGS. 5A-5H, in some examples, the attachment device 512 includes the housing fastening portion 514, which is an elongated member having a longitudinal axis L1 and an open proximal end extending over the open proximal end 524 of the digit housing 516. The housing fastening portion 514 can be oriented such that the longitudinal axis L1 of the housing fastening portion 514 is parallel with the longitudinal axis L2 of the digit housing 516. The elongated fastening portion 514 can be removably or permanently connected to the external digit housing surface 518 of the digit housing 516 by an adhesive or a mechanical connector. The skin securement portion 532 of the attachment device 512 is an elongated member having a longitudinal axis L3 (shown in FIGS. 5C and 5D) that extends transversely or substantially transversely (e.g., about 90 degrees) from a proximal end of the fastening portion 514. The skin securement portion 532 is configured to be transitioned from a pre-use position (shown in FIGS. 5C and 5D) to an in-use position (shown in FIGS. 5A and 5B), where ends of the skin securement portion 532 are attached together forming a ring, loop, or circular enclosure. As shown in FIGS. 5E-5H, the ring, loop, or circular enclosure can be secured around the third portion or proximal phalanges of the digit of the patient. In some examples, the skin securement portion 532 can include an adhesive for connecting ends of the skin securement portion together to form the ring, loop, or circular enclosure and/or for adhering to the third portion or proximal phalanges of the digit for securing the attachment device 512 to the digit.

In order to use the attachment device 512 of FIGS. 5A-5H, the patient or clinician first attaches the housing fastening portion 514 to the external digit housing surface 518 of the digit housing 516, such that the skin securement portion 532 is positioned proximal to the open proximal end 524 of the digit housing 516. The patient then inserts the digit to be monitored, such as the middle finger, into the digit housing 516, as shown in FIG. 5E. With the digit in the digit housing 516, the patient or clinician can attach ends of the skin securement portion 532 together around the third portion or proximal phalanges of the patient's digit forming the annular ring, loop, or circular enclosure, as shown in FIG. 5E.

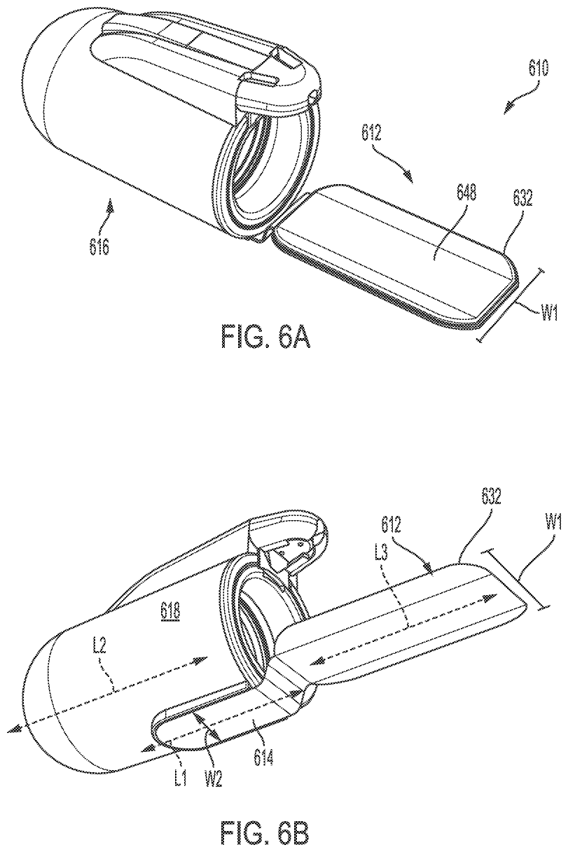

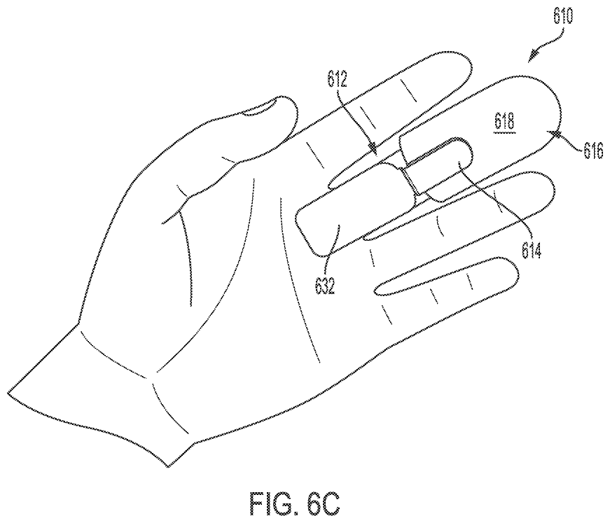

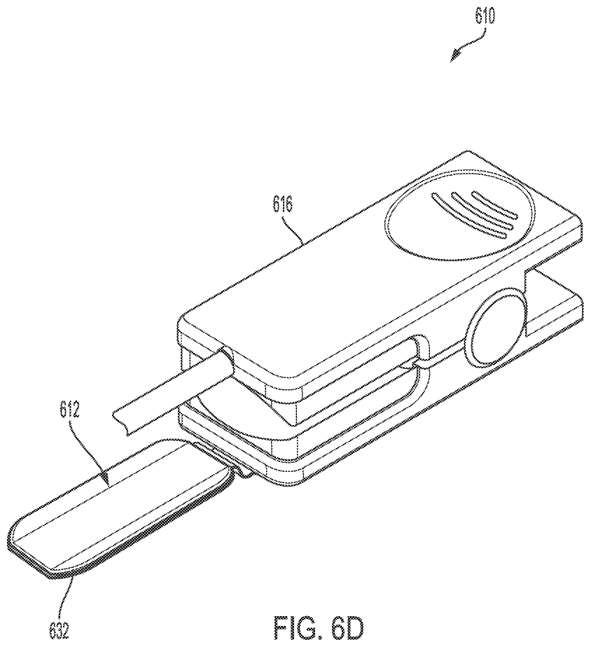

FIGS. 6A-6C show another example of the optical digit probe 610 including the digit housing 616 and the attachment device 612. Specifically, FIGS. 6A and 6B are perspective views of the optical digit probe 610 and the attachment device 612. FIG. 6C shows the optical digit probe 610 and the attachment device 612 attached to a digit of a patient.

As shown in FIGS. 6A-6D, the attachment device 612 includes the housing fastening portion 614 for securing the attachment device 612 to digit housing 616 and the skin securement portion 632 for securing the attachment device 612 to the third portion or proximal phalanges of the digit of the patient. However, unlike in previous examples, the attachment device 612 of FIGS. 6A-6D includes a skin securement portion 632 that is a longitudinally extending elongated member having a width W1 that is wider than a width W2 of the housing fastening portion 614. As shown in FIG. 6B, the longitudinal axis L1 of the housing fastening portion 614 and the longitudinal axis L2 of the digit housing 616 are parallel or substantially parallel (e.g., within about 10 degrees of parallel) with a longitudinal axis L3 of the skin securement portion 632. The fastening portion 614 of the attachment device 612 can be secured to the external digit housing surface 618 of the digit housing 616 in a same manner as in previous examples. For example, the fastening portion 614 can be adhered to the external surface 618 of the digit housing 616 by a hydrogel, a removable adhesive, or a permanent adhesive. In other examples, the fastening portion 614 can be connected to the external digit housing surface 618 of the digit housing 616 by mechanical fasteners, such as pins, screws, nails, staples, or thread (e.g., stitching). In other examples, the external digit housing surface 618 can comprise raised portions or protrusions sized to be received within openings or slots of the fastening portion 614 for connecting the housing fastening portion 614 to the external digit housing surface 618.

The skin securement portion 632 shown in FIGS. 6A-6D is an elongated member configured to be connected to the third portion or proximal phalanges of the digit, as shown in FIG. 6C. For some patients, the skin securement portion 632 may also extend proximally beyond the third portion or proximal phalanges of the digit. In such cases, the skin securement portion 632 can be configured to be adhered to or engage to the palm, top portion of the patient's hand, or other portions of the hand or wrist. In order to secure the skin securement portion 632 to the digit, hand, or wrist, the skin securement portion 632 can include an adhesive or adhesive layer 648 for adhering the skin securement portion 632 to the digit. As in previous examples, the adhesive layer 648 can be a hydrogel and/or removable adhesive, for removably coupling the attachment device 612 and digit housing 616 to the wearer.

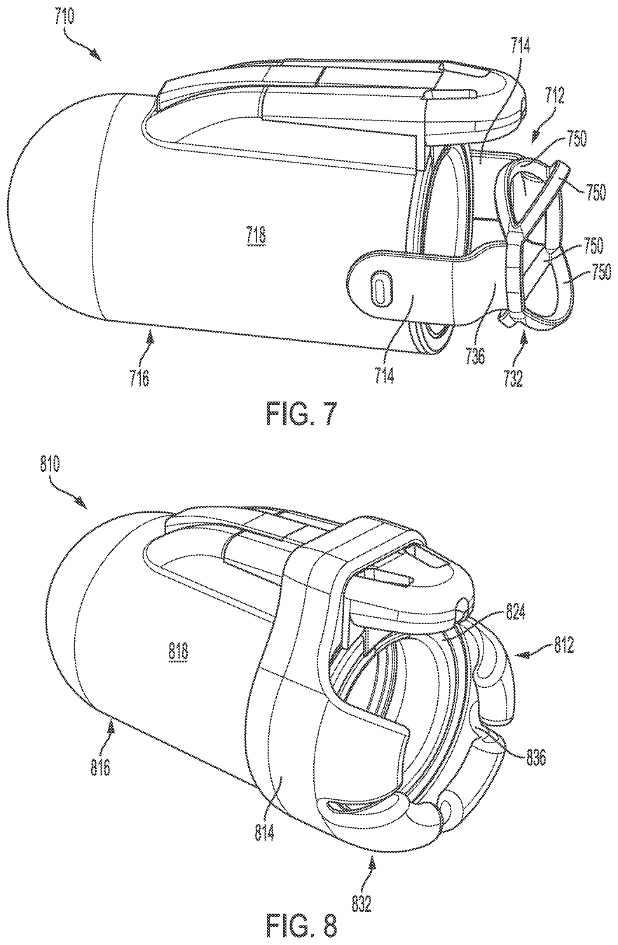

FIG. 7 is a perspective view showing another example of an optical digit probe 710 and attachment device 712. The attachment device 712 includes two housing fastening portions 714 that are connected to and extend proximally from the external digit housing surface 718 of the digit housing 716. The attachment device 712 also includes the skin securement portion 732 connected to and extending from proximal ends 736 of the two housing fastening portions 714. The skin securement portion 732 is a ring, loop, annular, or circular member positioned such that the wearer's digit is inserted through the ring and then through the open proximal end 724 of the digit housing 716. The ring or circular member of the skin securement portion 732 is configured to cinch or bias against the proximal portion of the patient's digit to hold the optical digit probe 710 on the digit. In order to enhance the connection with the proximal portion of the wearer's digit, the skin securement portion 732 can include inwardly biased members or segments 750 of the ring or circular member of the skin securement portion 732. The biased members or segments 750 are configured to press against the patient's digit enhancing the engagement between the optical digit probe 710 and the wearer's digit.

FIG. 8 is a perspective view showing another example of an optical digit probe 810 comprising a digit housing 816 and an attachment device 812. As in previous examples, the attachment device 812 can be permanently or removably attached to the external digit housing surface 818 of the digit housing 816. For example, the attachment device 812 can include a housing fastening portion 814 comprising an annular member or sleeve configured to be inserted over the open proximal end 824 of the digit housing 816. As in previous examples, the housing fastening portion 814 can comprise or can be formed from an elastomeric material, such as silicone, polypropylene, low molecular weight polyethylene, synthetic rubber (e.g., polychloroprene) or natural rubber (e.g., isoprene). The material can also be a high friction material that can engage the external surface 818 of the digit housing 816 creating a secure connection between the external digit housing surface 818 and an internal digit housing surface 820 of the housing fastening portion 814. In some examples, portions of the housing fastening portion 814 can be inwardly biased enhancing the engagement between the external digit housing surface 818 of the digit housing 816 and the fastening portion 814 of the attachment device 812.

The attachment device 812 also includes the skin securement portion 832. As shown in FIG. 8, the skin securement portion 832 includes inwardly angled or protruding fingers extending from a proximal end 836 of the annular member or sleeve over the open proximal end 824 of the digit housing 816. For example, as shown in FIG. 8, the skin securement portion 832 includes four protruding fingers of substantially similar shape and size. In other examples, the skin securement portion 832 can include fewer than or more than four protruding fingers. In some examples, the protruding fingers can be equidistantly spaced about a circumference of the annular member or sleeve of the housing fastening portion 814. In other examples, a majority of the protruding fingers can be positioned on one side of the annular sleeve, while an opposite side of the annular sleeve can have fewer protruding fingers or no protruding fingers.

In some examples, the protruding fingers are inwardly biased configured to press against the digit of the wearer when the digit is inserted through the protruding fingers and into an interior of the digit housing 816. Accordingly, the protruding fingers of the skin securement portion 832 can hold the digit housing 816 in place on the digit without using adhesives for holding the skin securement portion 832 against the digit housing 816.

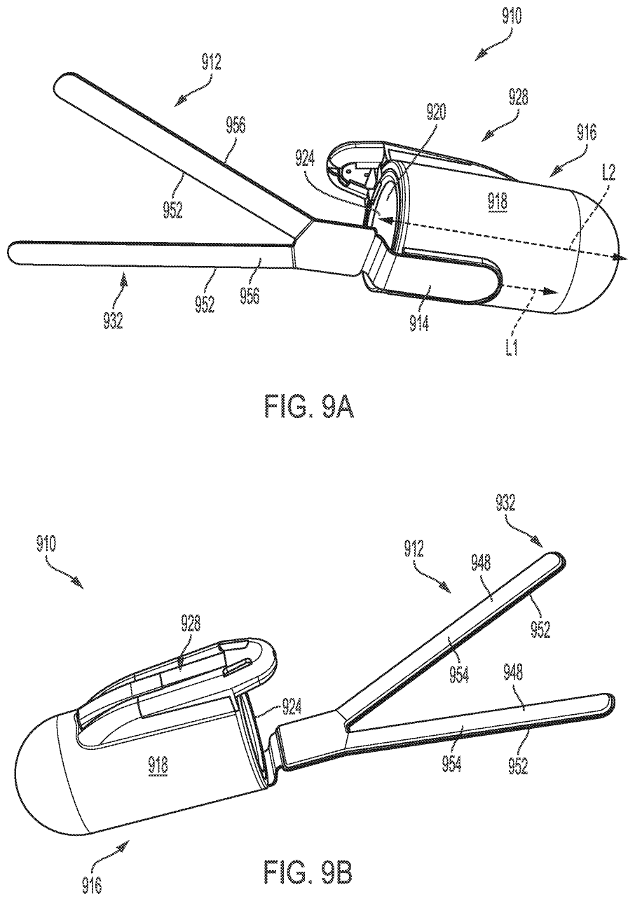

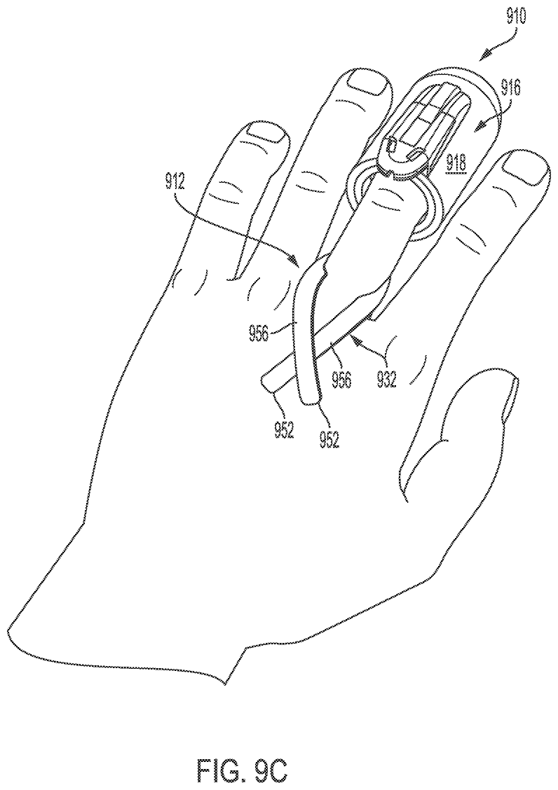

FIGS. 9A-9C show another example of an optical digit probe 910 including an attachment device 912 configured to be secured to a third portion or proximal phalanges of a digit of the patient. Specifically, FIGS. 9A and 9B are perspective views of the optical digit probe 910 and attachment device 912. FIG. 9C shows the optical digit probe 910 and attachment device 912 attached to a digit of a patient.

As in previous examples, the optical digit probe 910 includes the digit housing 916 comprising the external digit housing surface 918, the internal digit housing surface 920, and a proximal end 924 adapted for facilitating insertion of a digit of a patient into the digit housing 916. The optical digit probe 910 also includes the attachment device 912 for securing the optical digit probe 910 to a skin surface of the patient, as well as the optical sensor assembly 928 disposed in and/or on the digit housing 916 configured to be positioned proximate to the digit of the wearer or patient to provide optical signals reflecting the physiological parameter of the patient. For example, as previously described, the optical sensor assembly 928 can be positioned on a top portion of the digit housing 916, such that the optical sensor assembly 928 is above the digit inserted into the digit housing 916, as shown in FIG. 9C.

The optical digit probe 910 of FIGS. 9A-9C differs from previous examples in the configuration and appearance of the attachment device 912. Specifically, the attachment device 912 includes the housing fastening portion 914 connected to the external digit housing surface 918 of the digit housing 916. As in previous examples, the housing fastening portion 914 can be an elongated member that attaches to the external digit housing surface 918 of the digit housing 916 having a longitudinal axis L1 that is parallel or substantially parallel (e.g., within about 10 degrees of parallel) to a longitudinal axis L2 of the digit housing 916.

The attachment device 912 also includes a skin securement portion 932 extending from the housing fastening portion 914 configured to releasably couple the attachment device 912 to the skin surface of the patient about a third portion or proximal phalanges of the digit of the patient. The skin securement portion 932 of FIGS. 9A-9C includes two elongated members or tethers 952 extending from a proximal end of the housing fastening portion 914 at different angles forming a “v” shape, as shown in FIGS. 9A and 9B. The elongated members or tethers 952 are configured to be wrapped around the third portion or proximal phalanges of the digit of the patient for securing the skin securement portion 932 to the digit, as shown in FIG. 9C.

The elongated members or tethers 952 can be substantially flat members having an inwardly facing surface 954 configured to be secured to the digit and an outwardly facing surface 956 opposite the inwardly facing surface 954. As in previous examples, the inwardly facing surface 954 can be covered with a releasable or low-strength adhesive layer 948 for securing the skin securement portion 932 to the digit. The adhesive layer 948 can initially be covered by a protective sheet (not shown in FIGS. 9A and 9B), which can be removed prior to attaching the optical digit probe 910 to the digit of the patient. In order to attach the optical digit probe 910 to the digit of the patient, the patient first inserts the digit through the open proximal end 924 of the digit housing 916. The patient can then remove the protective cover or protective sheet from the tethers 952 of the skin securement portion 932 and can wrap the elongated members or tethers 952 around the third or proximal portion of the digit, as shown in FIG. 9C. In particular, proximal end portions of the elongated members or tethers 952 can be made to overlap, thereby forming a loop around the digit.

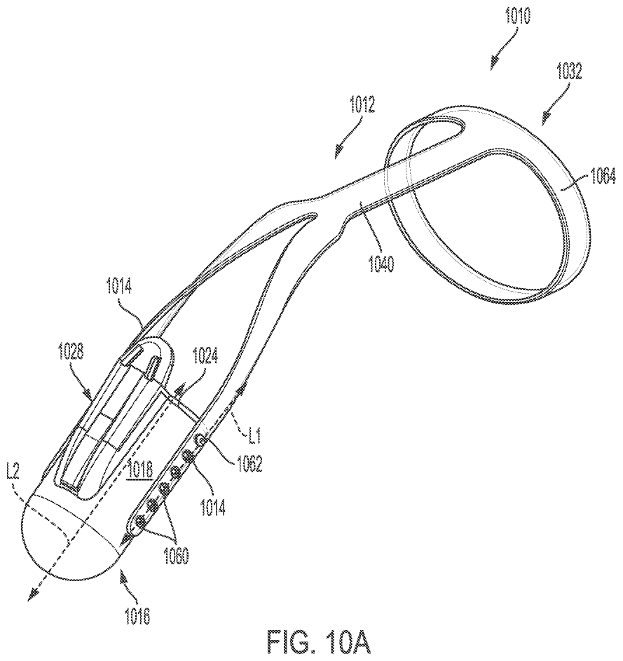

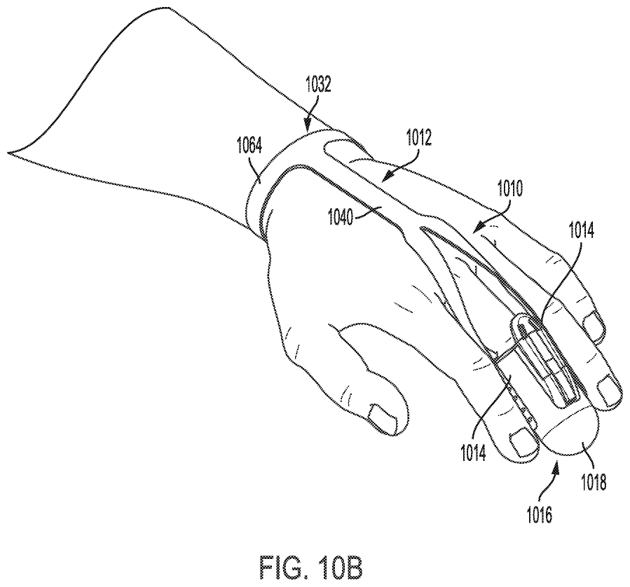

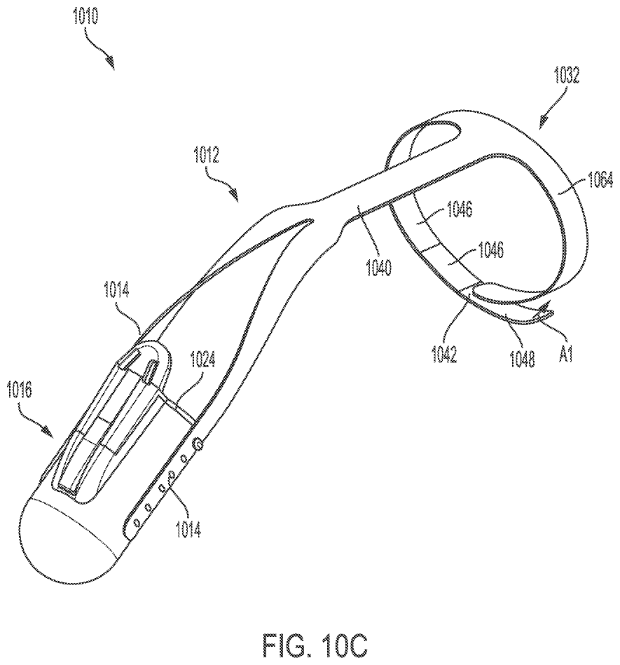

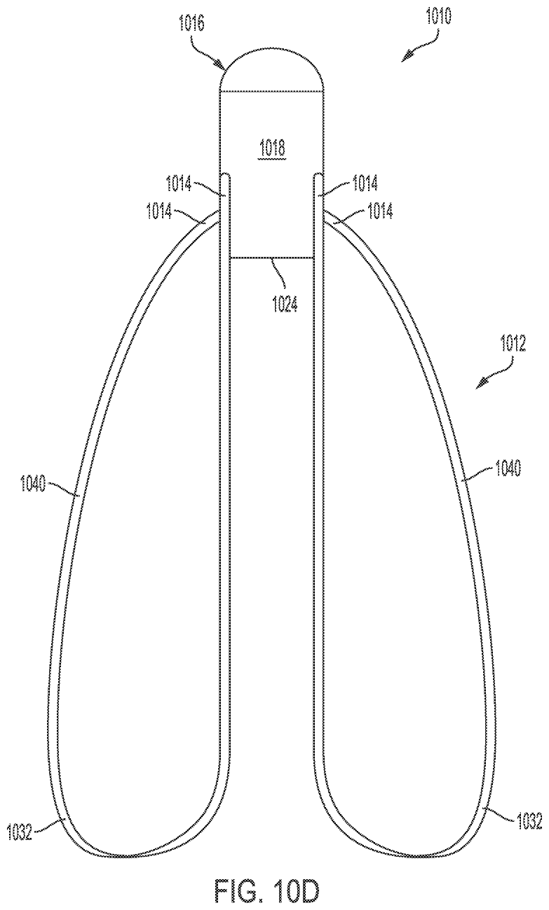

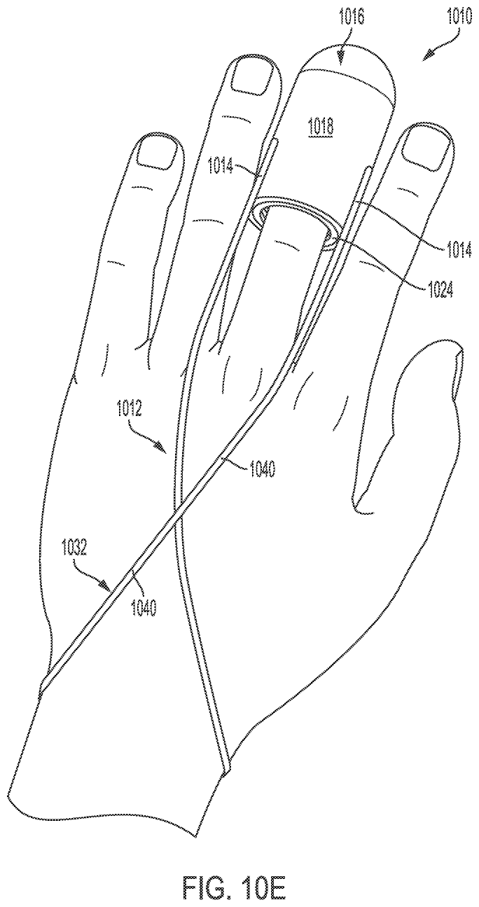

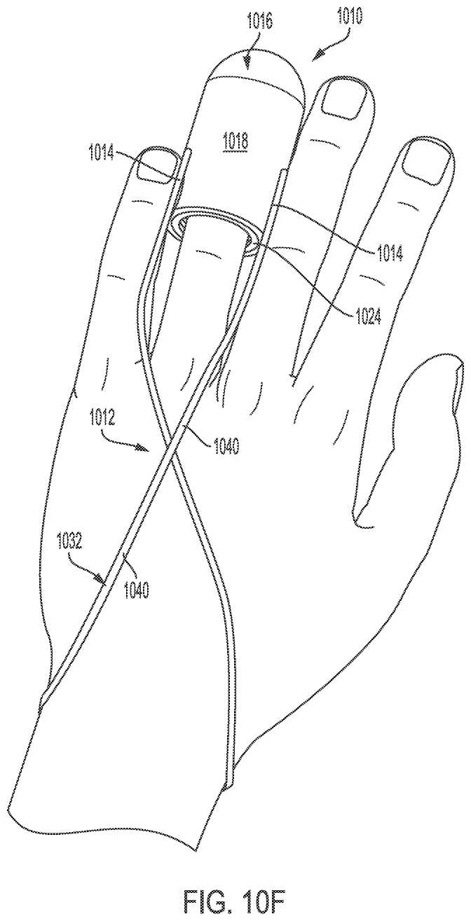

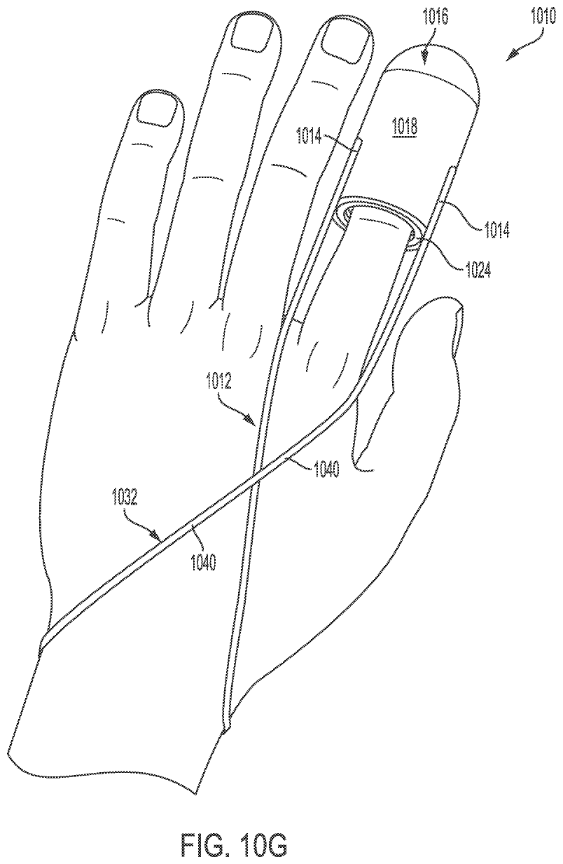

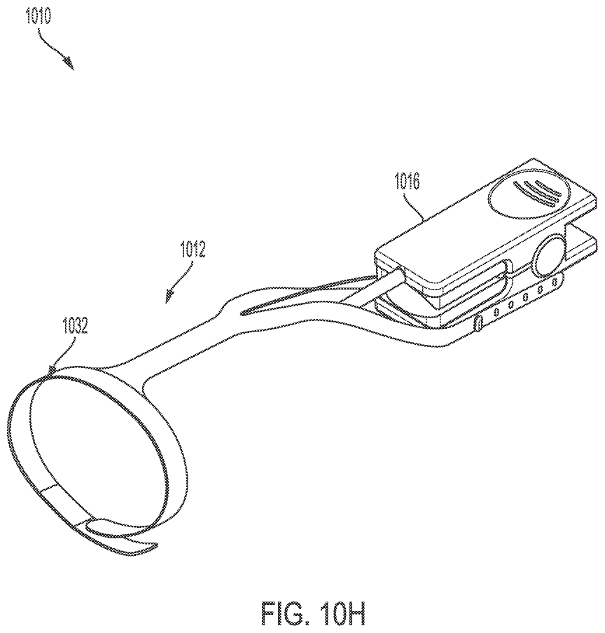

FIGS. 10A-10H show examples of an optical digit probe 1010 including an attachment device 1012 configured to be secured to or worn about a wrist of the patient. Specifically, FIG. 10A is a perspective view of the optical digit probe 1010 and attachment device 1012. FIG. 10B shows the optical digit probe 1010 and attachment device 1012 attached to a middle finger and wrist of a patient. FIG. 10C is a perspective view of another example of an optical digit probe 1010 and attachment device 1012 including an adhesive layer for securing the attachment device 1012 to the wrist portion of the patient. FIG. 10H is another example of an optical probe 1010 including the attachment device 1012 mounted to a clip-type probe housing 1016, which is similar or identical to the clip type probe of FIG. 2. FIGS. 10D-10G show another example of an optical digit probe 1010 including a different attachment device 1012 that secures the probe 1010 to the patient's wrist.

As in previous examples, the optical digit probes 1010 of FIGS. 10A-10G include the digit housing 1016 having an external digit housing surface 1018, an internal digit housing surface (not shown), and a proximal end 1024 adapted for facilitating insertion of a digit of the patient into the digit housing 1016. By contrast, the digit housing 1016 of the probe 1010 of FIG. 10H is the clip-type housing. The optical digit probe 1010 can be configured for insertion onto a middle finger of the patient as shown in FIG. 10B, as well as onto a ring finger (FIG. 10F) or a pointer or index finger (FIG. 10G). In other examples, the digit housing 1016 can be configured to be worn on a big toe (hallux) of the patient or on another convenient extremity of the patient. In other examples, the optical digit probe 1010 can be applied to other portions of the wearer's body, such as a lower arm of the wearer, an upper arm of the wearer, and/or a hand of the wearer.

Example dimensions of the digit housing 1016 depend upon the type of digit being monitored by the probe 1010. For an optical digit probe 1010 configured to be worn on a patient's middle finger, the housing 1016 can have an axial length of about 4 cm to about 8 cm. The digit housing 1016 can have an outer diameter of about 3 cm to about 4 cm and an inner diameter of about 2 cm to about 3 cm. In some implementations, the dimensions of the digit housing 1016 can be adjusted to accommodate varying digit sizes of the patient. For example, the inner diameter of the housing 1016 can vary within a range of about 2 cm to about 3 cm when the patient inserts his or her middle finger into the housing 1016. As noted, the dimensions depend on the patient's digit. In some implementations, the systems, methods, and devices as described herein can be configured to allow for fixation to any or all of the index finger (pointer finger or forefinger), middle finger, ring finger, little finger (pinky), or thumb. Accordingly, the dimensions may be different corresponding to the average person's dimensions for the foregoing patient digits.

The optical digit probe 1010 also includes the attachment device 1012, which comprises the housing fastening portion 1014 connected to the external digit housing surface 1018 of the digit housing 1016 and the skin securement portion 1032 configured to be releasably coupled to a skin surface of the patient. The optical digit probe 1010 also includes the optical sensor assembly 1028 disposed in or on the digit housing 1016 configured to be positioned proximate to the digit of the patient to provide optical signals reflecting physiological parameters of the patient. As previously described, the optical sensor assembly 1028 can be positioned on a top portion of the digit housing 1016, as shown in FIGS. 10A-10H, such that the optical sensor assembly 1028 is above the digit inserted into the digit housing 1016.

Unlike in previous examples, in which the skin securement portion of the attachment device connected to portion(s) of the digit (i.e., to third or proximal portions of the same digit that is inserted into the digit housing), the skin securement portion 1032 of FIGS. 10A-10H is configured to be worn on a wrist, forearm, and/or arm of the patient for releasably coupling the attachment device 1012 to the patient. For example, the skin securement portion 1032 can comprise a strap, ribbon, tether, sleeve, band, cuff, collar, bracelet, or similar wearable connector or anchor configured to be worn on the wrist, forearm, or arm of the patient for securing the optical digit probe 1010 in place and for preventing the digit housing 1016 from detaching from the digit of the patient at unexpected or inappropriate times. The attachment device 1012 can also include a middle portion 1040, such as a tether or strap, extending between the housing fastening portion 1014 and the skin securement portion 1032 of an appropriate length to extend from the patient's finger to the wrist, forearm, or arm of the patient. As shown in FIG. 10B, the middle portion 1040, such as the tether or strap, can be configured to rest against an outwardly facing side of the patient's hand when the optical digit probe 1010 is worn by the patient.

In some examples, as shown in FIGS. 10A and 10B, the attachment device 1012 includes two housing fastening portions 1014 removably or permanently connected, for example, on opposite sides of the external digit housing surface 1018 of the digit housing 1016. The two housing fastening portions 1014 can be elongated members or straps having a longitudinal axis L1 that is substantially parallel to a longitudinal axis L2 of the digit housing 1016. The elongated members or straps of the housing fastening portions 1014 can include holes, openings, slots 1060, or other recesses configured to receive corresponding protrusions 1062 extending from the external digit housing surface 1018 of the digit housing 1016 for securing the attachment device 1012 to the digit housing 1016.

As shown in FIGS. 10A and 10B, the skin securement portion 1032 of the attachment device 1012 is an annular or partially annular bracelet 1064 configured to be worn about the wrist of the patient. The bracelet 1064 can be formed from a stretchable and/or elastomeric material so that the bracelet 1064 can expand, allowing the bracelet 1064 to be used for patients with substantial variability in wrist size. In addition, in some examples, the bracelet 1064 can include a clasp, lock, buckle, or another resizing mechanism for resizing the bracelet 1064 to provide additional size adjustability for the bracelet 1064.

FIG. 10C shows another example of an optical digit probe 1010 including the attachment device 1012 comprising the housing fastening portion 1014 and the skin securement portion 1032 configured to be worn on a wrist, forearm, and/or arm of the patient for releasably coupling the attachment device 1012 to the wrist, forearm, or arm of the patient. The attachment device 1012 of FIG. 10C includes an adhesive layer 1048 on an inwardly facing surface 1042 of the attachment device 1012. The adhesive layer 1048 is covered by multiple segments of a protective cover 1046, which can be removed to expose the adhesive layer 1048.

As shown in FIG. 10C, the attachment device 1012 also includes the two housing fastening portions 1014 attached to opposing sides of the external digit housing surface 1018 of the digit housing 1016, as in the previous example. The housing fastening portions 1014 are attached to the external digit housing surface 1018 by the adhesive layer 1048. In order to attach the housing fastening portions 1014 to the external digit housing surface 1018, the patient or clinician removes segments of the protective cover 1046 on the housing fastening portions 1014 of the attachment device 1012 from the adhesive layer 1048 and then presses the exposed portions of the adhesive layer 1048 against the external digit housing surface 1018 of the digit housing 1016, thereby securing the housing fastening portions 1014 to the external digit housing surface 1018.

As shown in FIG. 10C, the skin securement portion 1032 of the attachment device 1012 includes an elongated member configured to be formed into a cuff or bracelet 1064 that can be worn on the patient's wrist, forearm, or arm. In order to form the bracelet 1064, the patient or clinician removes one or more segments of the protective cover 1046 from the inwardly facing surface 1042 of the attachment device 1012 to expose the adhesive layer 1048. The patient or clinician then presses the exposed portion of the adhesive layer 1048 against another portion of the skin securement portion 1032, as shown by arrow A1 in FIG. 10C, thereby forming the bracelet 1064. Forming the bracelet 1064 in this manner means that the bracelet 1064 can be sized for a particular patient. For patients with thin or narrow wrists, more of the adhesive layer 1048 can be exposed and attached to other portions of the skin securement portion 1032, thereby forming a narrow bracelet 1064 with a small inner diameter. For wearers with larger or wider wrists, less of the adhesive layer 1048 can be exposed, thereby forming a wider bracelet 1064 with a larger inner diameter.

In order to attach the optical digit probe 1010 to the digit of the patient, a clinician or the patient obtains the digit housing 1016 and the removable attachment device 1012. The patient or clinician can then attach the two housing fastening portions 1014 to the external digit housing surface 1018 of the digit housing 1016, as previously described, by removing segments of the protective cover 1046 from the adhesive layer 1048 and pressing the adhesive layer 1048 against the external digit housing surface 1018 of the digit housing 1016. After the housing fastening portions 1014 are attached to the digit housing 1016, the patient can insert the digit, such as a middle finger, into the digit housing 1016 through the open proximal end 1024 of the digit housing 1016. The patient can then place the skin securement portion 1032 of the attachment device 1012 over his or her wrist, such that the middle portion 1040 of the attachment device 1012 extends over the outwardly facing side of the patient's hand. Then the clinician or patient can remove segments of the proactive cover 1046 from the adhesive layer 1048 near a proximal end of the skin securement portion 1032 and attach the exposed portions of the adhesive layer 1048 to another portion of the skin securement portion 1032 to form the bracelet 1064 or loop. As previously described, the bracelet 1064 or loop extends around the patient's wrist, which secures the digit housing 1016 and attachment device 1012 in place on the digit and wrist of the patient.

FIGS. 10D-10G show another example of an optical digit probe 1010 including a skin securement portion 1032 configured to be secured to a wrist, forearm, or arm of a patient. Specifically, FIG. 10D is a front view of the optical digit probe 1010 and the attachment device 1012. FIG. 10E shows the optical digit probe 1010 and attachment device 1012 attached to a middle finger and hand or wrist of a patient. FIG. 10F shows the optical digit probe 1010 and attachment device 1012 attached to a ring finger of a patient. FIG. 10G shows the optical digit probe 1010 and attachment device 1012 attached to a pointer or index finger of a patient.

As in previous examples, the optical digit probe 1010 of FIGS. 10D-10G includes the digit housing 1016 and the attachment device 1012. The attachment device 1012 includes the housing fastening portion 1014 connected to the external digit housing surface 1018 of the digit housing 1016. As in previous examples, the housing fastening portion 1014 of FIG. 10D can be attached to the digit housing 1016 by an engagement between holes or slots of the housing fastening portion 1014 and protrusions extending from the external digit housing surface 1018 of the digit housing 1016, as shown in FIGS. 10A and 10B. Alternatively, the housing fastening portion 1014 can be connected to the digit housing 1016 by an adhesive, as shown in FIG. 10C.

The attachment device 1012 of FIGS. 10D-10G also includes the skin securement portion 1032 extending proximally from the housing fastening portions 1014. The housing fastening portions 1014 and the skin securement portion 1032 are straps comprising a first end and a second end connected to the external digit housing surface 1018 of the digit housing 1016. For example, the attachment device 1012 can include two straps removably or permanently connected to opposing sides of the external digit housing surface 1018 of the digit housing 1016 by a mechanical fastener and/or adhesive. The straps can include an elongated member or portion between the first end and the second end configured to be wrapped over a palm and around at least one of a wrist, forearm, and/or arm of the patient.

In order to attach the optical digit probe 1012 of FIGS. 10D-10G to the digit of the patient, such as to a middle finger, ring finger, or pointer/index finger of the patient, the patient inserts the digit into the digit housing 1016 through the open proximal end 1024 of the digit housing 1016. The clinician or patient then moves the straps of the housing fastening portion 1014 and skin securement portion 1032 around the patient's hand and wrist, thereby securing the digit housing 1016 of the optical digit probe 1010 to the digit of the patient. For example, as shown in FIGS. 10E-10G, the straps of the housing fastening portion 1014 and skin securement portion 1032 can be positioned over the inwardly facing surface or palm of the patient's hand and the outwardly facing surface of the patient's hand, such that a middle portion 1040 of each strap is positioned proximate to the patient's wrist. The straps can cross on the inwardly facing surface and/or outwardly facing surface of the hand forming an “X” as shown in FIGS. 10E-10G.

FIG. 10H shows the attachment device 1012 of FIG. 10C attached to a clip-type optical probe or digit housing 1016. As previously described in connection with FIG. 2, the clip-type probe or digit housing 1016 comprises a clip formed from upper and lower elongated trays or bodies pivotally connected together at a distal end thereof. The digit to be monitored can be inserted between the elongated bodies or trays, and the elongated bodies or trays can be permitted to pivot towards each other, thereby securing the housing 1016 to the digit to be monitored. As shown in FIG. 10H the attachment device 1012 includes housing fastening portions connected to the bottom tray of the housing 1016 and a skin securement portion 1032 configured to be worn on a wrist, forearm, and/or arm of the patient. The attachment device 1012 also includes an adhesive layer on an inwardly facing surface of the attachment device 1012 for securing ends of the skin securement portion 1032 together to form the loop or bracelet sized to be worn on the patient's wrist.

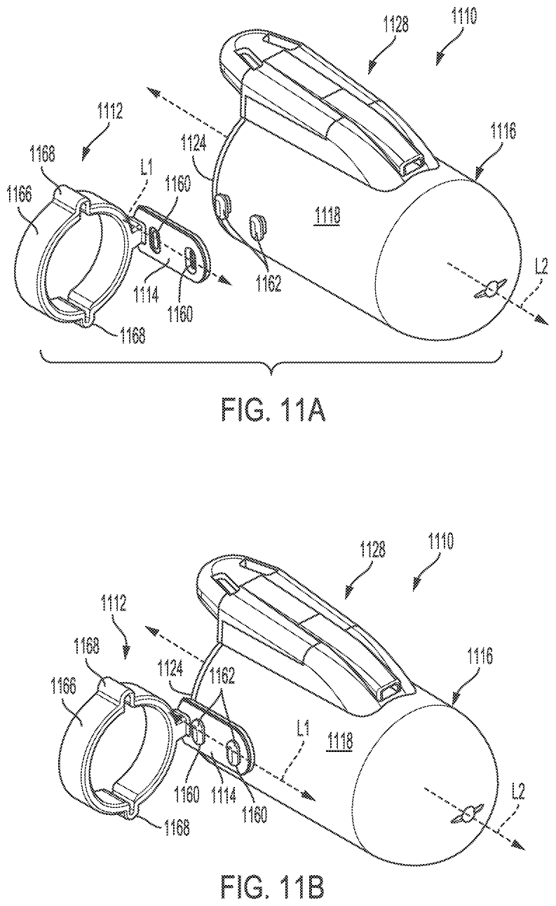

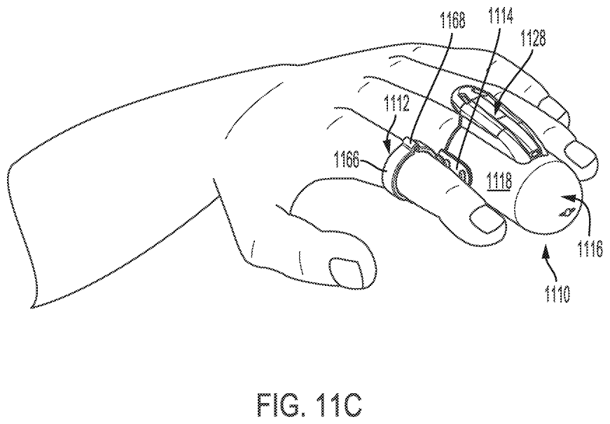

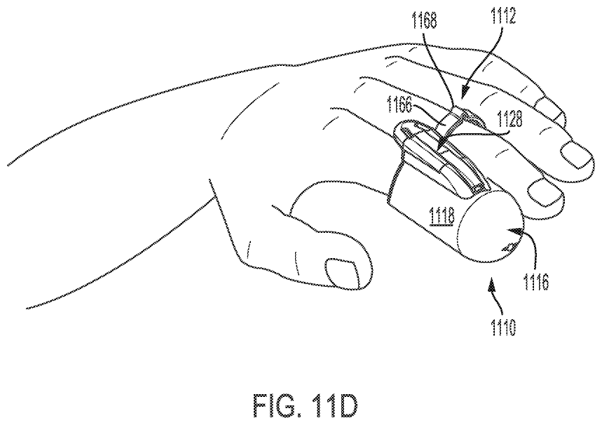

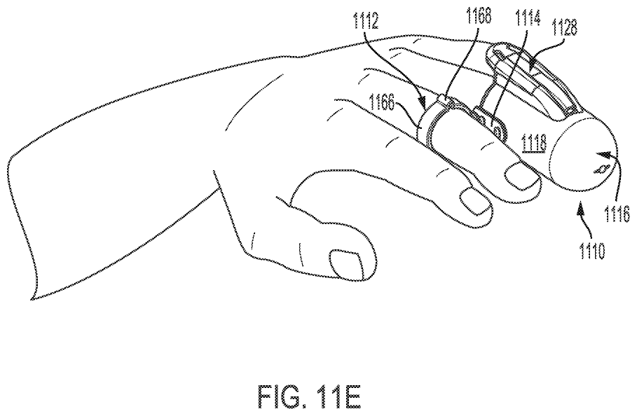

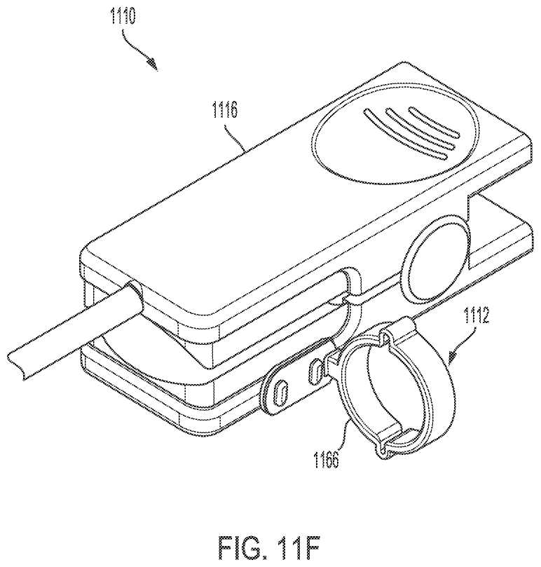

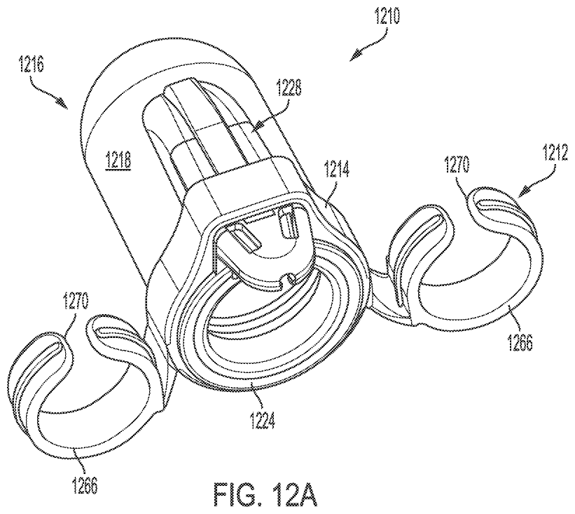

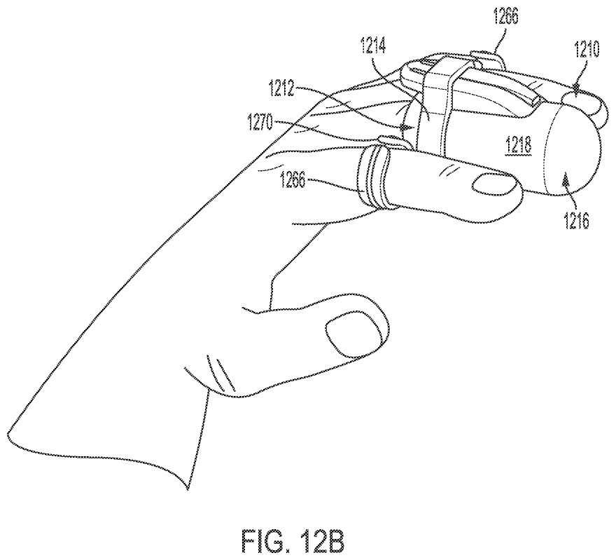

FIGS. 11A-12B show additional examples of an optical finger probe 1110, 1210 including the digit housing 1116, 1216 and the attachment device 1112, 1212 connected to an external digit housing surface 1118, 1218 of the digit housing 1116, 1216. As in previous examples, the digit housing 1116, 1216 comprises the external digit housing surface 1118, 1218, the internal digit housing surface 1118, 1218 and the proximal end 1124, 1224 adapted for facilitating insertion of a digit of the patient into the digit housing 1116, 1216. The optical digit probe 1110, 1210 also includes the optical sensor assembly 1128, 1228 disposed in or on the digit housing 1116, 1216 comprising the optical sensor configured to be positioned proximate to the digit of the patient to provide optical signals reflecting a physiological parameter of the patient. In some examples, the optical digit probe 1110, 1210 can be configured for insertion onto a middle finger of the patient. In other examples, the digit housing 1116, 1216 can be configured to be worn on a big toe (hallux) of the patient or on another convenient extremity of the patient. In other examples, the optical digit probe 1110, 1210 can be applied to other portions of the wearer's body, such as a wrist of the wearer, a lower arm of the wearer, an upper arm of the wearer, and/or a hand of the wearer.

Example dimensions of the digit housing 1116, 1216 depend upon the type of digit being monitored by the probe 1110, 1210. For an optical digit probe 1110, 1210 configured to be worn on a patient's middle finger, the digit housing 1116, 1216 can have an axial length of about 4 cm to about 8 cm. The digit housing 1116, 1216 can have an outer diameter of about 3 cm to about 4 cm and an inner diameter of about 2 cm to about 3 cm. In some implementations, the dimensions of the digit housing 1116, 1216 can be adjusted to accommodate varying digit sizes of the patient. For example, the inner diameter of the housing 1116, 1216 can vary within a range of about 2 cm to about 3 cm when the patient inserts his or her middle finger into the housing 1116, 1216. As noted, the dimensions depend on the patient's digit. In some implementations, the systems, methods, and devices as described herein can be configured to allow for fixation to any or all of the index finger (pointer finger or forefinger), middle finger, ring finger, little finger (pinky), or thumb. Accordingly, the dimensions may be different corresponding to the average person's dimensions for the foregoing patient digits.

As in previous examples, the attachment device 1112, 1212 comprises the housing fastening portion 1114, 1214 that is permanently or removably connected to the external digit housing surface 1118, 1218 of the digit housing 1116, 1216. For example, as shown in FIGS. 11A-11E, the housing fastening portion 1114 can be an elongated member including one or multiple slots 1160, holes, or openings configured to receive corresponding protrusions 1162 for securing the housing fastening portion 1114 to the external digit housing surface 1118 of the digit housing 1116. When connected to the housing 1116, the housing fastening portion 1114 can be oriented with a longitudinal axis L1 of the housing fastening portion 1114 parallel or substantially parallel (e.g., within about 10 degrees of parallel) to a longitudinal axis L2 of the digit housing 1116.

Unlike in previous examples, the attachment devices 1112, 1212 of FIGS. 11A-12B also include one or more anchor portions 1166, 1266 configured to be secured to a digit of the patient that is different from the digit inserted into the digit housing 1116, 1216, thereby removably coupling the attachment device 1112, 1212 to the patient. For example, as shown in FIGS. 11C and 12B, the digit housing 1116, 1216 is connected to a middle finger of the patient and the anchor portion 1166, 1266 is connected to an index finger and/or ring finger of the patient. Alternatively, as shown in FIG. 11D, the digit housing 1116 can be connected to a pointer or index finger of a patient and the anchor portion 1166 can be connected to a middle finger of the patient. In another example, as shown in FIG. 11E, the digit housing 1116 can be connected to a ring finger of a patient and the anchor portion 1166 can be connected to a middle finger of the patient.

In some examples, as shown in FIGS. 11A-11E, the attachment device 1112 includes a single anchor portion 1166 connected to a single housing fastening portion 1114. The single anchor portion 1166 can be an expandable ring including two size-expanding sections 1168 positioned on opposite sides of the ring configured to unfold so that the ring can expand, meaning that the ring can be worn by patients with wider digits (e.g., fingers). Also, as in previous examples, the ring can be formed from a stretchable and/or elastomeric material which can stretch to accommodate larger digits.

In order to apply the optical digit probe 1110 of FIGS. 11A-11E to a digit of a patient, the patient or clinician attaches the attachment device 1112 to the external digit housing surface 1118 of the digit housing 1116 by, for example, inserting the protrusions 1162 extending from the external digit housing surface 1118 through the openings or slots 1160 of the housing fastening portion 1114. The patient then inserts the digit housing 1116 onto the digit being sensed or measured and simultaneously inserts an adjacent or neighboring digit through the anchor portion 1166 for securing the optical digit probe 1110 in place on the digit. For example, as previously described and as shown in FIG. 11B, the digit housing 1116 can be inserted onto the middle finger of the patient while the anchor portion 1166 slides onto the patient's index finger.

FIG. 11F shows the attachment device 1112 of FIGS. 11A-11E attached to a clip-type optical probe or digit housing 1116. As previously described in connection with FIG. 2, the clip-type probe or digit housing 1116 comprises a clip formed from upper and lower elongated trays or bodies pivotally connected together at a distal end thereof. As shown in FIG. 11F the attachment device 1112 includes a housing fastening portion connected to the bottom tray of the housing 1016 for securing the attachment device 1112 to the housing 1116. The attachment device 1012 also includes an anchor portion 1166 extending from the housing fastening portion configured to be secured to a digit of the patient that is different from the digit that is received in the digit housing 1116, thereby removably coupling the attachment device 1112 and digit housing 1116 to the patient.

As shown in FIGS. 12A and 12B, in other examples, the attachment device 1212 of the optical digit probe 1210 includes two anchor portions 1266 configured to be attached to digits adjacent to the digit inserted into the digit housing 1216. More specifically, as shown in FIG. 12A, the attachment device 1212 comprises an annular housing fastening portion 1214 that is inserted over the proximal end 1224 of the digit housing 1216, such that an inner surface of the annular housing fastening portion 1214 contacts the external digit housing surface 1218 of the digit housing 1216, thereby securing the attachment device 1212 to the digit housing 1216.

The attachment device 1212 also includes the two anchor portions 1266 connected to and extending radially outward from the annular housing fastening portion 1214. As shown in FIG. 12A, the anchor portions 1266 are partial rings sized to receive digits of the patient adjacent to the digit inserted into the digit housing 1216. For example, a middle finger of the patient can be inserted into the digit housing 1216. As shown in FIG. 12B, a ring finger of the patient can be inserted through on of the anchor portions 1266 and an index finger of the patient can be inserted through the other anchor portion 1266. In some examples, the anchor portions 1266 can include a slot, gap, or opening 1270 allowing the anchor portions 1266 to be stretched or deformed to fit around larger fingers or digits. Alternatively, in other examples, the anchor portions 1266 can include size-adjustable segments, clamps, buckles, folded portions, or other structural features allowing for resizing of the anchor portions 1266 to accommodate digits of different sizes. Also, the anchor portions 1266 and/or other portions of the attachment device 1212 can be formed from stretchable and/or elastomeric materials to increase adjustability of the anchor portions 1266.

In order to attach the attachment device 1212 to a digit of a patient, the clinician or wearer first inserts the open proximal end 1224 of the digit housing 1216 into the annular housing fastening portion 1214, thereby securing the digit housing 1216 to the attachment device 1212. With the attachment device 1212 suitably and securely connected to the digit housing 1216, a digit to be measurement and monitored, such as a patient's middle finger, is inserted into the digit housing 1216 through the open proximal end 1224 of the digit housing 1216. Simultaneously, digits adjacent to the inserted digit are received in the anchor portions 1266 of the attachment device 1212, thereby securing the attachment device 1212 to the digits of the patient. For example, as previously described, the ring finger of the patient can be inserted though one of the anchor portions 1266 and the index finger of the patient can be inserted through the other anchor portion 1266, thereby securing the optical digit probe 1210 to the digits of the patient.

Having described the optical digit probes and attachment devices of the present disclosure in detail, features of medical systems and devices 110 for monitoring patient physiological parameters will now be described. The medical systems and devices 110 can include any of the previously described optical digit probes configured to be attached to a digit, such as a finger of the patient, and attachment devices for securing the optical digital probes to the patient's digit. As previously described, the optical digit probes 112 can include the optical sensors and/or other physiological sensors for detecting signals representative of physiological parameters of the patient. The medical systems and devices 110 disclosed herein can also include other sensors and devices for obtaining physiological information for a patient and for transmitting the obtaining information from the medical systems and device 110 to remote computer devices, servers, and networks.

FIGS. 13A-13C and FIG. 14 show features of an exemplary medical device 110 comprising the optical digit probe 112 for noninvasive measurement of physiological parameters of a patient. The patient can be, for example, a patient undergoing a medical test, such as an at home medical test. For example, the patient can be undergoing a home or remote sleep apnea test, in which optical digit probe 112 is worn on a digit of the wearer overnight while the wearer is sleeping. In other examples, the patient can be undergoing tests for other aspects of patient respiration or other respiratory conditions. The tests can be multiple hours in duration. The wearable medical device 110 and optical digit probe 112 can also be worn by a patient in a medical facility for continuous and ongoing monitoring of respiratory parameters of the patient over a period of hours or days.

As shown in FIGS. 13A-13C, the wearable medical device 110 includes the optical digit probe 112. In some examples, the optical digit probe 112 can be an oximeter for obtaining an oxygen saturation measurement for the patient. The optical digit probe 112 can also be a pneumatic probe configured to be mounted to a digit of a patient for providing continuous measurements for a relative state of vasomotor activity in a distal part of the patient's digit based on a plethysmography method.

As previously described, the optical digit probe 112 can be configured to cover the distal part of the digit of the patient (shown in FIG. 13C) with a uniform predetermined pressure field extending to the tip of the digit. The optical digit probe 112 can be adjustable, so that it can be used with many or all possible digit dimensions. Also, the optical digit probe 112 can be configured to avoid venous blood pooling or engorgement and stasis, which inhibits retrograde venous shock wave propagation. In this way, the optical digit probe 112 can be configured to allow for partial unloading of arterial wall tension, which significantly improves a dynamic range of the measured signal. The optic component or optical sensor 130 of the optical digit probe 112 can measure optical density related changes of arterial blood volume in the digital arteries, associated with each heartbeat. Peripheral arterial constrictions, when present, are shown by attenuation in signal amplitude of a detected signal, which is a marker of sympathetic activation.

In some examples, the optical digit probe 112 comprises a digit housing 114 formed, for example, from a rigid material, such as a rigid plastic (e.g., acrylonitrile butadiene styrene (ABS), polyester, polycarbonate, polypropylene, polyethylene, or polyethylene terephthalate). The digit housing 114 includes a closed distal end 124, an open proximal end 116, and a tubular or cylindrical sidewall 128 extending between the proximal end and the distal end. The optical digit probe 112 can also include sealing structures, such as an internal O-ring or cushion, positioned to secure provide a tight seal around a digit of a wearer that is inserted into an interior of the housing.

The digit housing 114 can also include structures for providing mechanical support for electronic components of the optical digit probe 112, such as a circuit board and/or computer processor, which can be in electronic communication with an optical sensor 130 disposed on or in the digit housing 114. In particular, the electronic components and associated circuitry can comprise electronics circuits for receiving and/or processing signals representative of photoplethysmography (PPG) and/or oxygen saturation arterial hemoglobin (SpO₂) measurements and for transmitting the received and processed signals to other devices through a connection cable 134. The optical sensor 130 and associated electronic circuitry can be positioned in an optical sensor assembly 132 positioned on a top portion of the digit housing 114, such that the optical sensor assembly 132 is above the digit inserted into the digit housing 114. The optical sensor assembly 132 can also include a connector that receives the wire or connection cable 134 for connecting the optical digit probe 112 to other devices or components of the medical device 110 or system. The optical digit probe 112 can also include one or more membranes positioned within the digit housing 114 configured to provide pressure against the digit inserted into the housing. One or more of the inner membranes can be formed from a flexible protective material that prevents moisture or other materials from passing through the membrane to other portions of the optical digit probe 112. For example, the protective membranes could be formed from an elastomeric materials, such as nitrile rubber, synthetic rubber, latex, or similar materials. The inner membranes can be configured to provide unified pressure on the digit of the wearer by, for example, air trapped between two of the inner membranes and/or between the membranes and the digit housing 114 of the optical digit probe 112.

The optical digit probe 112 also includes the optical sensor 130 in electronic communication with the electronic components positioned on the circuit board. The optical sensor 130 can be configured to detect and monitor PPG and/or pulse oximetry signals. More specifically, signal(s) measured by the optical sensor 130 can be signals representative of pulsatile volume changes in arteries of a digit (e.g., in a fingertip of the wearer) that reflect a relative state of the arterial vasomotor activity, which relate indirectly to a level of sympathetic activation. Peripheral arterial vasoconstriction, which mirrors sympathetic activation, can be shown as attenuation of signal amplitude for signals detected by the optical sensor 130. In some examples, the optical digit probe 112 can also include sensors 136 for measuring infrared signals (e.g., RED and IR (Infra-Red) signals), which can also be used for the measurement of the pulse oximetry (SpO₂) signal. Specifically, in some examples, the optical sensor 130 and/or infrared (IR) sensor 136 can be configured to measure changes in absorbance of the digit (e.g., finger) at both red and infrared light at peak wavelengths of approximately 660 nm and 910 nm, respectively, and having a maximum optical output power of about 65 mW. Measurements from the optical sensor 130 and the IR sensor 136 can be used to calculate the oximetry signal according to the pulse oximetry principles.

In some examples, the optical digit probe 112 includes or is in electronic communication with a controller 138 (shown in FIG. 13B), such as a computer processor. The controller 138 can be configured to control acquisition of the physiological signals from the optical sensor 130 or IR sensor 136 and/or to control the transmission of data (e.g., data collected during a sleep study) based on physiological signals detected by the sensors 130, 136. As previously described, the physiological signals can include signals for detecting PPG, blood pressure, and other physiological parameter values for the wearer or patient.

With continued reference to FIGS. 13A-13C, the medical device 110 can also include the wearable monitor, such as the wrist-worn monitor 118. The wrist-worn monitor 118 can be a battery-powered monitoring device, which can be worn on a wrist, forearm, arm, or another convenient location of the wearer. The wrist-worn monitor can be electrically connected to the optical digit probe 112 by the connecting cable 134. The wrist-worn monitor 118 can comprise electronical circuitry of the medical device 110 including the controller 138. The wrist-worn monitor 118 can also include a wireless transceiver 140 or communications module for wireless transmission of captured and/or processed data from the medical device 110 to a remote computer device or computer server.

In some examples, the wrist-worn monitor 118 also includes one or more physiological sensors 148 (shown in FIG. 13B) for detecting additional physiological information for the patient. The sensor 148 can include a PPG sensor comprising, for example, at least one light emitting device (e.g., a photoemitter) and at least one light detecting device (e.g., a photodetector). The wrist-worn monitor 118 can also include multiple PPG sensors 148, such as a plurality of light emitting devices (e.g., 3 light emitting devices having different wavelengths) and a light detecting device (e.g., configured to detect light of each wavelength of the light emitting devices). The wrist-worn monitor 118 can also include other sensors 150, such as position or motion sensors (e.g., a body position sensor or an arm position sensor). In other examples, the sensor 150 can be a heart or blood flow sensor for measuring pulse, ECG, blood pressure, and/or cardiac physiological parameters.

In some examples, the medical device 110 further comprises the chest motion sensor device 120 configured to be positioned on a chest of the patient. As shown in FIGS. 13A-13C, the chest motion sensor device 120 can be connected to the wrist-worn monitor 118 by a second connecting cable 152 and can be configured to provide sensed data to the controller 138 of the wrist-worn monitor 118 for processing and/or to the wireless transceiver 140 of the wrist-worn monitor 118 for transmission to the remote computer device or server. The chest motion sensor device 120 can comprise a housing 142, such as a rigid plastic housing, enclosing circuitry for detecting movement and acoustic signals in proximity to the wearer. Signals detected by sensors of the chest motion sensor device 120 can be related to snoring, body position, and/or the wearer's chest movement. The housing 142 can comprise an adhesive surface or adhesive layer on a bottom portion of the housing 142, which can be configured to be adhered to the chest of the patient to maintaining positioning of the chest motion sensor device 120 of the wearer's chest for obtaining sensor data from sensors of the chest motion sensor device 120. As shown in FIG. 13B, in some examples, the chest motion sensor device 120 comprises an acoustic snore sensor 144 and a chest movement sensor 146. The snore sensor 144 can be an acoustic decibel detector comprising, for example, a highly sensitive microphone that responds to snoring and other sounds in the audio range. The snore sensor 144 can be configured to convert detected sounds to a signal that provides a reliable indication that such sounds have been detected. The chest movement sensor 146 can be an accelerometer, such as a 3-axis accelerometer, that provides a signal that reflects the movement of the chest, which can be translated, for example, both to the patient's sleeping posture (supine, prone, right, left and sit) and to the chest movement signal resulted by the subject's breathing during the night.

In some examples, as previously described, the medical device 110 can be configured to transmit, such as via the wireless transceiver 140 of the wrist-worn monitor 118, medical data, such as medical information based on signals detected on sensors 130, 136 of the optical digit probe 112 and/or signals detected by sensors 144, 146 of the chest motion sensor device 120. In such an example, the wireless connection may include at least one of the following: a cellular connection, a Bluetooth connection, Advanced Message Queuing Protocol (AMQP) connection, Constrained Application Protocol (CoAP) connection, a WiFi connection, a ZigBee connection, a Z-Wave connection, a wireless personal area network (WPAN) connection, an Infrared Data Association (IrDA) connection, or any combination thereof.

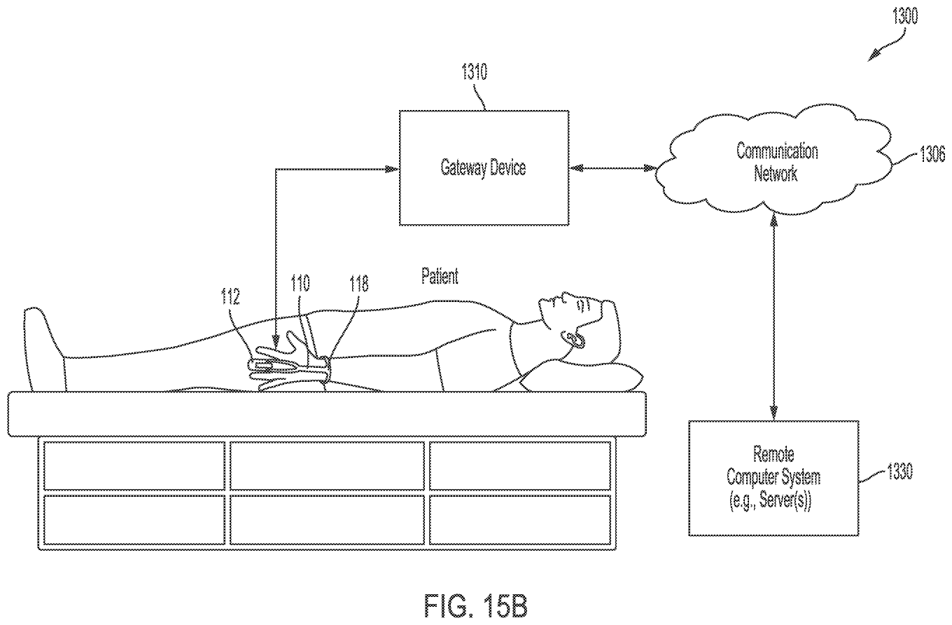

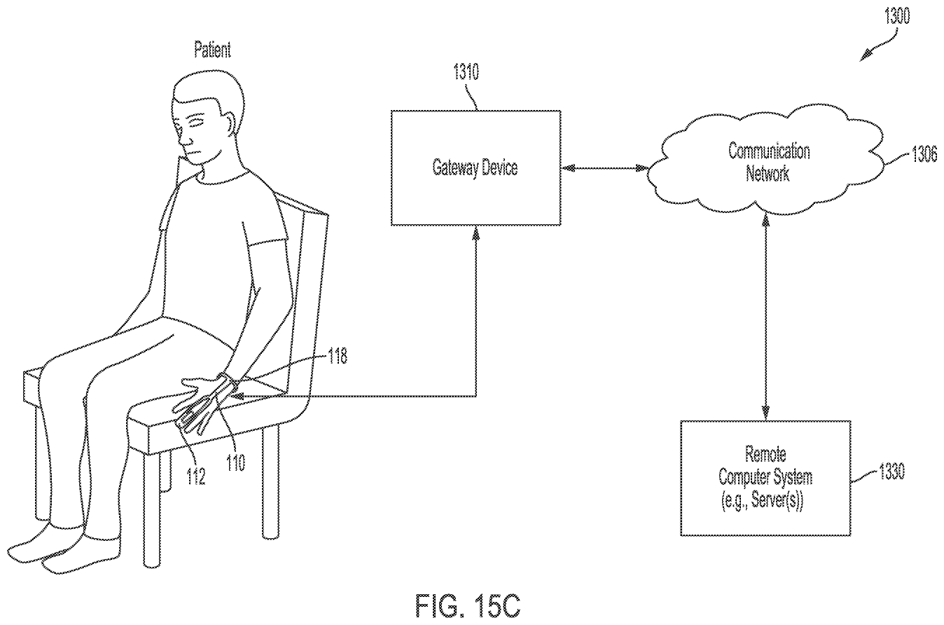

As described in further detail in connection with FIGS. 15A-15C, signals and/or data detected and/or processed by the optical digit probe 112, wrist-worn monitor 118, and/or chest motion sensor device 120 can be transmitted, via the wireless transmitter 140, to remote servers for further processing and for preparing reports summarizing collected data. For example, data can be wirelessly transmitter from the medical device 110 to an intermediate or gateway device, such as a mobile phone running a data collection application. The mobile phone can be configured to store received data in memory and to transmit the received data to the remote server on a continuous or ongoing basis.

Once data is received by the remote server, the server can execute software to analyze the received data using various automatic algorithms for detecting respiratory and other events that occurred during sleep, as well as periods of REM, deep sleep, light sleep and wakefulness. A pulse rate signal can be derived from the received data and used in the automatic analysis. The received data, such as night data from the sleep study, can be viewed by a technician and, if required, automatically detected events can be revised manually. The received analyzed data can then be used to generate reports, such as comprehensive reports of a patient sleep study including statistics and graphic presentations of recorded data and test results. Once generated, the comprehensive report(s) including the overnight sleep study data can be stored in Web Server storage and delivered to interested parties (e.g., a prescribing physician, a caregiver, or the patient) via the Internet.

Having generally described the various devices and sensors of the medical device 110, specific features of the sensors 130, 136 of the optical digit probe 112 will not be described in further detail, with reference to FIG. 14. FIG. 14 is a schematic drawing showing how the optical sensor 130 and/or IR sensor 136 of the optical digit probe 112 emit radiation pulses and record reflective signals representative of the physiological parameters of the wearer or patient.

As shown in FIG. 14, an optical digit probe 201a, which can be used with the medical device 110 and the attachment device of the present disclosure, can be tubular, with a closed end and an open end, such that, when the digit of the wearer or patient is placed therein, a distal part of the digit (e.g., a fingertip) is closest to the closed end, and a proximal part of the digit is closest to the open end of optical digit probe 201a. More specifically, the optical digit probe 201a can be sized to enclose about a distal-most two-third portion of the digit. The proximal third of the digit can be outside of the optical digit probe 201a.

In some examples, as previously described, the optical digit probe 201a comprises a pneumo-optical sensor, which may include a light emitting device 222 and a light detecting device 224. For example, the light emitting device 222 may include at least one of a light emitting diode (LED), a photoemitter, a laser, any combination thereof, and/or the like. The light detecting device 224 may include at least one of a photodiode, a photodetector, a photosensor, a photoresistor, a semiconductor based photodetector, any combination thereof, and/or the like. In some embodiments, the light emitting device 222 may have a wavelength of about 550 nm (e.g., substantially visible green), about 650 nm (e.g., substantially visible red), 660 nm, about 780 nm (e.g., substantially infrared), 800 nm, 910 nm, 940 nm, and/or the like. In some examples, the optical digit probe 201a may also include other physiological sensors (not pictured) as described in connection with FIGS. 13A-13C.

In some examples, the optical digit probe 201a can include a plurality of light emitting devices 222. For example, each respective light emitting device 222 may have a respective wavelength. In some examples, the respective wavelength of each respective light emitting device 222 may be different than the wavelengths of at least some of (e.g., all of) the other light emitting devices 222. For example, a first light emitting device 222 may have a first wavelength (e.g., about 650 nm, about 660 nm, and/or the like) and a second light emitting device 222 may have a second wavelength (e.g., about 550 nm). In some examples, a single light detecting device 224 may detect light from all light emitting devices 222. In some embodiments, optical digit probe 201a may include a plurality of light detecting devices 224, each respective light detecting device 224 corresponding to (e.g., configured to detect the respective wavelength of) a wavelength of at least one of the light emitting devices 222.

As previously described, in some examples, an inner portion of the optical digit probe 201a can include a uniform pressure field. For example, a uniform pressure field may be achieved by inserting an elastic member 226 into the open end of optical digit probe 201a and attaching the elastic member 226 to the open end of optical digit probe 201a such that an enclosed seal is formed between an inner portion of the optical digit probe 201a and the elastic member 226 to define a pocket 228. A fluid (such as an inert gas) may be inserted (e.g., injected and/or the like) into the pocket 228 and cause the elastic member 226 to elastically deform. For example, the pneumo-optical sensor may be utilized in conjunction with the elastic member 226 to apply the uniform subdiastolic pressure to the portion of the patient's body (e.g., to a distal two-thirds of the digit, such as a fingertip of the wearer's finger, etc.), whereby the uniform subdiastolic pressure may one or more of: clamp the finger probe to the wearer's finger, facilitate unloading of arterial wall tension, facilitate increase in a dynamic range of a peripheral arterial signal of the patient relative to PPG signals without the uniform subdiastolic pressure, and/or mitigate distal venous pooling or distention to avoid induction of venoarterial-mediated vasoconstriction.

In some examples, the uniform pressure field may be static and, when utilized in conjunction with the light emitting device 222 and the light detecting device 224, may produce a peripheral arterial signal or measurement, which may be more sensitive than conventional PPG measurements. Further, the static uniform pressure field may inhibit or prevent pooling of venous blood in the distal end of the digit while allowing pulsatile blood delivered by the arteries to be returned via the veins. In some examples, the pressure applied by the uniform pressure field may be sufficient to prevent free venous flow due to, for example, hydrostatic pressure and shock waves, while allowing the veins to carry blood delivered by the arteries out of the finger. In some examples, the pressure required to prevent venous pooling may differ from patient to patient.

In some examples, noise reduction (e.g., improved signal to noise ratio) in the peripheral arterial signal or measurement may be achieved by applying sufficient pressure to partially unload, but not occlude, the wall tension of the arteries in the finger, when the finger is near heart level. This may allow the arterial wall to move freely to accommodate the pulsatile blood delivery of the heart. The applied pressure may be slightly above the maximum pressure in the veins when the hand is fully lowered (e.g., 5% higher and/or the like).

In order to apply the optical digit probe 201a to an appendage, such as a finger, pressure (e.g., the uniform pressure field) may be applied so that optical digit probe 201a does not move. Additionally or alternatively, an adhesive may be used to secure the optical digit probe 201a in place, and/or positioning members may attach the optical digit probe 201a to a wristband, a watch band, and/or the like (e.g., to the wrist-worn monitor 118 shown in FIGS. 13A-13C).

Having described the medical device 110 and optical digit probe 112, as well as the various exemplary attachment devices used to secure the optical digit probe 112 to the digit of the patient, physiological monitoring systems 1300 for controlling the optical digit probe 112 and/or for collecting, storing, analyzing, and providing feedback about physiological information detected by the optical digit probe 112 and/or the medical device 110 will now be described in detail. Exemplary physiological monitoring systems 1300 for collecting and analyzing data collected by the optical digit probe 112 and associated medical devices 110 are shown in FIGS. 15A-15C. Specifically, FIGS. 15A-15C are schematic drawings and block diagrams of the system 1300 showing communication pathways for transmitting detected information and other data from the optical digit probe 112 and medical device 110 to remote computer devices or computer servers. Further, FIG. 15B shows a patient lying on a bed wearing the medical device 110 and optical digit probe 112, as may occur during an overnight sleep study. FIG. 15C shows an awake patient sitting in a chair while wearing the medical device 110 and the optical digit probe 112, as may occur when the probe 112 is used for periodic or continuous monitoring of patient physiological parameters.

As shown in FIGS. 15A-15C, the system 1300 comprises a remote monitoring device 1302 (shown in FIG. 15A) in communication with the medical device 110. For example, the remote monitoring device 1302 can be in communication with the controller of the wrist-worn monitor 118 and/or with other electronic or monitoring circuitry of the medical device 110 for receiving information detected by sensors of the optical digit probe 112 and/or other medical devices 110. The system 1300 can also include a wireless communication network 1306, and/or a wired connection 1308 for transmitting information, such as data detected by sensors of the optical digit probe 112 and/or medical device 110, from the medical device 110 to the remote monitoring device 1302.

Devices of the wireless communications network 1306 can include one or more of the following types of communication circuitry: cellular communications circuitry, Bluetooth® communications circuitry, Advanced Message Queuing Protocol (AMQP) circuitry, Constrained Application Protocol (CoAP) circuitry, WiFi circuitry, ZigBee circuitry, Z-Wave circuitry, wireless personal area network (WPAN) circuitry, Infrared Data Association (IrDA) circuitry, or any combination thereof. For example, wireless communications circuitry of the remote monitoring device 1302 may be configured to establish at least one of the following types of wireless connections with the medical device 110 and/or optical digit probe 112: a cellular connection, a Bluetooth® connection, an Advanced Message Queuing Protocol (AMQP) connection, a Constrained Application Protocol (CoAP) connection, a WiFi connection, a ZigBee connection, a Z-Wave connection, a wireless personal area network (WPAN) connection, an Infrared Data Association (IrDA) connection, or any combination thereof.

In some examples, the remote monitoring device 1302 can include one or more devices capable of receiving information from and/or communicating information to the medical device 110 and/or to other computer servers (not shown in FIG. 15A) (e.g., via the wireless communication network 1306, via wired connection 1308, etc.). In some examples, the remote monitoring device 1302 can include a server or a group of servers. Additionally or alternatively, the remote monitoring device 1302 can include at least one other computing device separate from or including the server and/or group of servers, such as a portable and/or handheld device (e.g., a computer, a laptop, a personal digital assistant (PDA), a smartphone, a tablet, and/or the like), a desktop computer, and/or other like devices, as described herein. In some examples, the remote monitoring device 1302 may include at least one network interface (e.g., a server network interface and/or the like), at least one data storage device (e.g., a server database and/or the like), at least one processor (e.g., a server processor and/or the like), any combination thereof, and/or the like. For example, the remote monitoring device 1302 may include at least one processor operatively connected to a non-transitory computer-readable medium. In some examples, the remote monitoring device 1302 can be in communication with at least one data storage device (e.g., a server database and/or the like), which may be local or remote to the remote monitoring device 1302. In some examples, the remote monitoring device 1302 may be capable of receiving information from, storing information in, communicating information to, or searching information stored in the data storage device (e.g., a server database and/or the like).

In some examples, the system 1300 can be configured for conducting a home and/or remote sleep study using the optical digit probe 112 and medical device 110 of the present disclosure. In particular, as shown in FIGS. 15B and 15C, the wrist-worn monitor 118 can be strapped to a wrist of a patient and the optical digit probe 112 can secured to the patient's digit or finger. For example, as previously described, the attachment devices disclosed herein can be used to secure the optical digit probe 112 to the digit of the patient ensuring that the optical digit probe 112 does not become dislodged during the home and/or remote sleep study. As previously described, the wrist-worn monitor 118 can be configured to receive PPG signals, as well as a peripheral arterial signal and/or oxygen saturation data from the optical digit probe 112. The wrist-worn monitor 118 can also receive actigraphy (movement) data from the chest motion sensor device 120 (not shown in FIGS. 15A-15C) and transmit the relevant data using an application running on a mobile device, such as the gateway device 1310, via the communication network 1306 to a remote computer system 1330 (e.g., one or more web servers) for further processing.

The system 1300 depicted in FIGS. 15A-15C is useful for conducting a home and/or remote sleep study, such as an overnight sleep study. For example, as shown in FIG. 15B, the patient may wear the medical device 110 and the optical digit probe 112 while sleeping in a bed. Data such as physiological data obtained from the sleep study can be stored on the remote computer system 1330 and obtained, preferably after conclusion of the sleep study, for evaluation. The data may include respiratory and other events that occurred during sleep as well as periods of REM, deep sleep, light sleep and wakefulness. The pulse rate signal may be derived from the peripheral arterial signal and used in the automatic analysis. For example, the system 1300 can be configured to generate various parameters, including but not limited to: a respiratory disturbance index, an apnea-hypopnea index, a central apnea-hypopnea index, a percentage of total sleep time with Cheyne-Stokes Respiration pattern and sleep staging identification. A report may be generated, and the relevant data of the sleep study may be viewed on a screen and the automatically detected events can be revised manually by a physician as needed.

As previously described, the optical digit probes 112 and medical devices 110 disclosed herein can be used for obtaining physiological measurements for a patient remote from a medical facility, such as at home. For example, as previously described, the optical digit probes 112 and medical devices 110 disclosed herein can be used for Home and/or Remote Sleep Apnea Tests (e.g., “HSATs”). In order to guide patients in performing such at home tests, the patient can be provided with verbal or written instructions for performing the at home study. For example, the instructions can guide the patient in how to correctly remove the optical digit probe 112 from packaging and prepare the probe 112 for use. The instructions may also include guidance for how to attach the probe 112 to the patient's digit and/or for using the attachment device to secure the probe 112 in place on the digit. The instructions can also include guidance on how long the probe 112 should be worn and/or on how to transmit collected data from the medical device 110 to the remote server after the study has been completed.

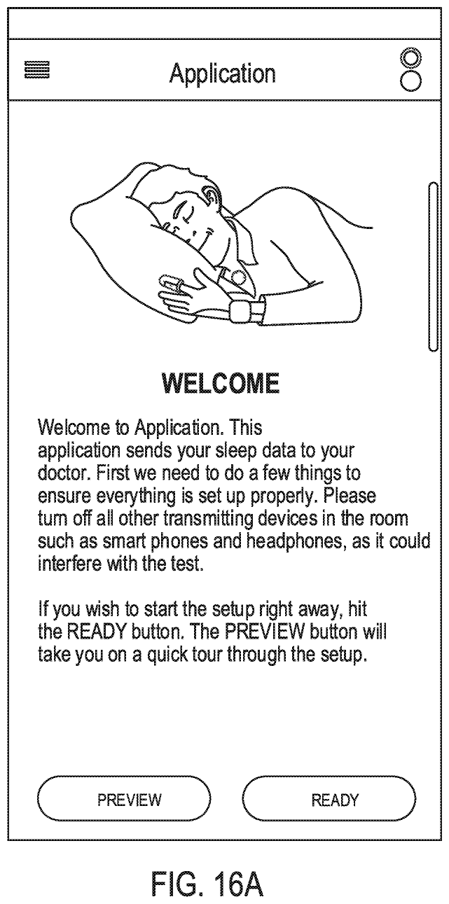

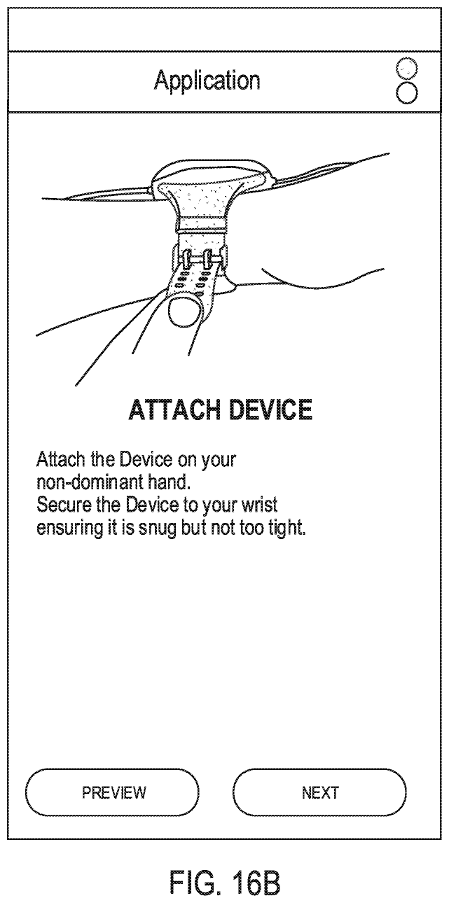

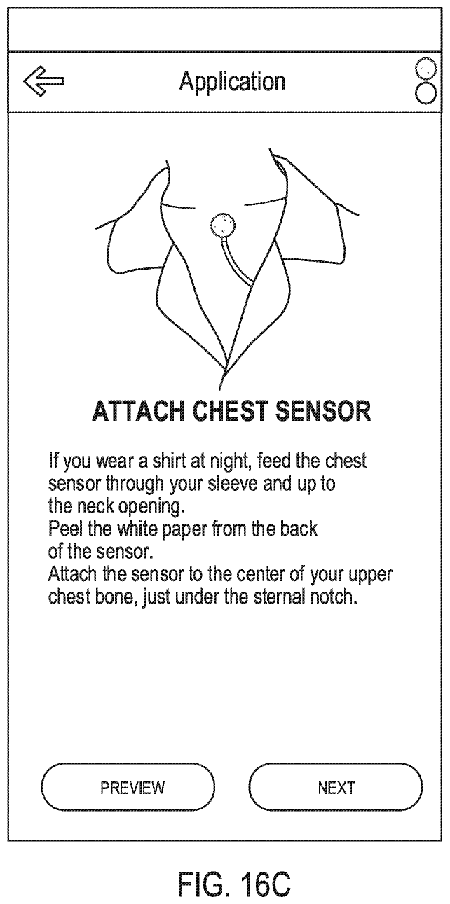

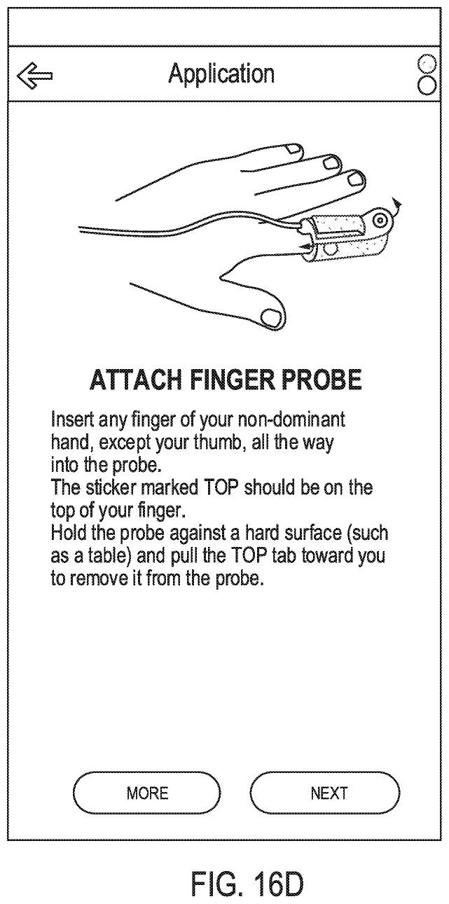

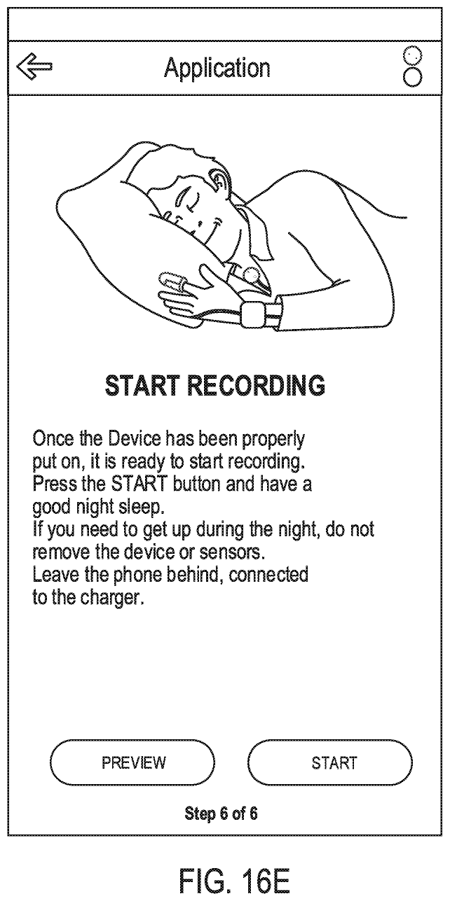

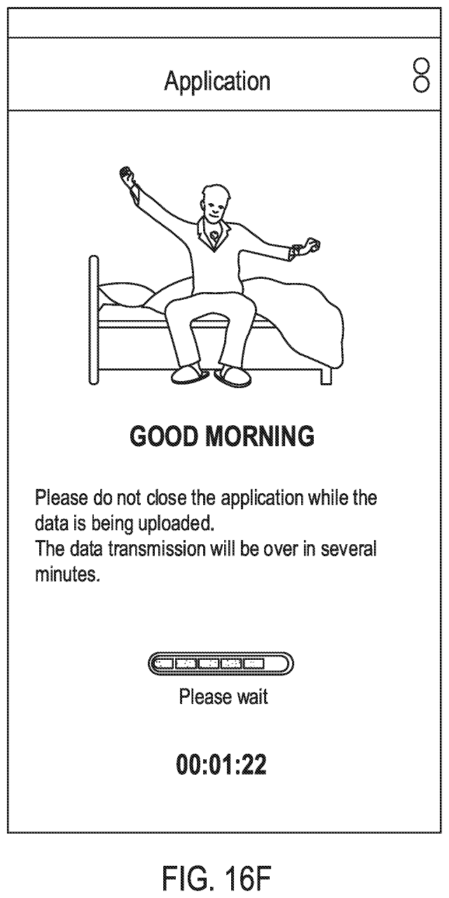

In some examples, instructions can be provided on an electronic device, such as a patient's smart phone or personal computer. Examples of instruction screens or a user interface for guiding the patient in performing an HSAT at-home study using the optical digit probe 112 and medical device 110 are shown in FIGS. 16A-16F. Specifically, FIG. 16A is a Welcome Screen explaining to the patient the type of study that will be performed. FIG. 16B is an instruction screen showing the patient how to attach the wrist-worn monitor to his or her wrist. FIG. 16C is an instruction screen showing the patient how to attach the chest motion sensor device to his or her chest. FIG. 16D is an instruction screen showing the patient how to attach the optical digit probe 112 or finger probe to his or her finger. Specifically, as shown in FIG. 16D, the patient is instructed to attach the probe 112 to any finger of his or her non-dominant hand. FIG. 16E is a Start Recording screen providing a virtual button for the patient to press when he or she is ready to begin recording sleep study data. Finally, FIG. 16F is a Good Morning or Study Completed Screen informing the patient that the sleep study has been completed and that recorded data is being transmitted from the medical device 110 to the remote computer server.

Although various non-limiting examples of the invention have been described in detail for the purpose of illustration based on what is currently considered to be the most practical and preferred aspects, it is to be understood that such detail is solely for that purpose and that the invention is not limited to the disclosed examples, but, on the contrary, is intended to cover modifications and equivalent arrangements that are within the spirit and scope of the appended claims. For example, it is to be understood that the present disclosure contemplates that, to the extent possible, one or more features of any example can be combined with one or more features of any other aspect or example.

1. An optical digit probe for noninvasive measurement of at least one physiological parameter of a patient, the optical digit probe comprising:
a digit housing comprising an external digit housing surface and an internal digit housing surface, wherein a proximal end of the digit housing is adapted for facilitating insertion of a digit of the patient into the digit housing;
an attachment device comprising:
a housing fastening portion connected to the external digit housing surface of the digit housing, and
a skin securement portion extending from the housing fastening portion configured to releasably couple the attachment device to a skin surface of the patient and to resist movement of the digit out of the digit housing, with resistance by the skin securement portion to movement of the digit out of the digit housing being greater than resistance by the skin securement portion to movement of the digit into the digit housing; and
at least one optical sensor disposed in the digit housing configured to be positioned proximate to the digit of the patient to provide optical signals reflecting the at least one physiological parameter of the patient.

2. The optical digit probe of claim 1, wherein the optical digit probe is configured for non-invasive measurement of arterial vasomotor activity through the digit of the patient.

3. The optical digit probe of claim 1, wherein the optical signals provided by the at least one optical sensor reflect an arterial pulse waveform of the patient.

4. The optical digit probe of claim 1, wherein the attachment device comprises a longitudinal attachment device having a longitudinal axis corresponding to an insertion direction of the digit of the patient into the digit housing.

5. The optical digit probe of claim 1, wherein the housing fastening portion is on a distal end of the attachment device and the skin securement portion is on a proximal end of the attachment device, the attachment device further comprising a middle portion between the housing fastening portion and the skin securement portion.

6. The optical digit probe of claim 4, wherein the longitudinal attachment device is configured to be permanently fastened to the digit housing.

7. (canceled)

8. The optical digit probe of claim 1, wherein the digit housing comprises a pressure device configured to apply a substantially uniform sub-diastolic pressure field to the digit of the patient, and
wherein the pressure device is configured to at least one of facilitate unloading of arterial wall tension, mitigate distal venous pooling, or distention to avoid induction of venoarterial-mediated vasoconstriction.

9-10. (canceled)

11. The optical digit probe of claim 1, wherein the attachment device further comprises:
an attachment portion extending between the housing fastening portion of the external digit housing surface of the digit housing portion and the skin securement portion.

12-14. (canceled)

15. The optical digit probe of claim 11, wherein the digit housing portion, the skin securement portion, and the attachment portion are integrally formed by a single molding process.

16-32. (canceled)

33. The optical digit probe of claim 1, wherein the proximal end of the digit housing is adapted for facilitating insertion of at least portions of a first portion and a second portion of a digit of the patient into the digit housing, and wherein the skin securement portion is configured to releasably couple the attachment device to a skin surface about a third portion of the digit of the patient.

34-68. (canceled)

69. The optical digit probe of claim 1, wherein the skin securement portion is configured to releasably couple the digit housing to the digit.

70-73. (canceled)

74. The optical digit probe of claim 69, wherein the optical digit probe comprises a longitudinal axis, and the skin securement portion comprises a plurality of flexible members arranged as flaps in a transverse plane about a center, and wherein in an assembled configuration in which the skin securement portion is detachably coupled to the digit housing, the transverse plane is intersected by the longitudinal axis.

75-76. (canceled)

77. The optical digit probe of claim 74, wherein in the assembled configuration, one or more of the flexible members comprises an outer base portion and an inner tip portion, and in a relaxed state extends along a radial axis in the transverse plane.

78-80. (canceled)

81. The optical digit probe of claim 74, wherein the plurality of flexible members are configured to bend and contact the digit of the patient along the digit when the digit is distally inserted into the open proximal end of the digit housing.

82. The optical digit probe of claim 74, wherein the skin securement portion defines a plurality of slots bordering the plurality of flexible members.

83. The optical digit probe of claim 82, wherein at least one of the plurality of flexible members is bordered by a respective pair of adjacent slots of the plurality of slots, and
wherein the skin securement portion defines a central hole in communication with the plurality of slots.

84-89. (canceled)

90. The optical digit probe of claim 69, wherein the skin securement portion comprises one or more grip members operatively disposed inside the digit housing and configured to grip the digit of the patient.

91-95. (canceled)

96. An attachment device for releasably coupling a digit of a patient to an optical digit probe configured for noninvasive measurement of at least one physiological parameter associated with the patient, the optical digit probe having a first housing portion which houses an optical sensor assembly, and a second housing portion which comprises an open proximal end, the attachment device comprising:
a housing fastening portion configured to detachably couple to at least one of the first housing portion or the second housing portion; and
a skin securement portion configured to detachably couple to the housing fastening portion, allow distal translation of the digit of the patient into the optical digit probe, and resist proximal translation of the digit of the patient out of the optical digit probe.

97. The attachment device of claim 96, wherein the housing fastening portion comprises an engagement portion configured to receive and detachably couple to the second housing portion to rotatably fix the housing fastening portion relative to the optical sensor assembly.

98. (canceled)

99. The attachment device of claim 96, wherein the attachment device comprises a longitudinal axis, the skin securement portion comprises a plurality of flexible members, and in an assembled configuration in which the skin securement portion is detachably coupled to the housing fastening portion, the plurality of flexible members extend in a direction transverse to the longitudinal axis of the attachment device.

100-104. (canceled)
